(19) United States
(12) Patent Application Publication
(10) Pub. No.: US 2026/0035475 A1
(43) Pub. Date: Feb. 5, 2026

(54) STABILIZED BINDING MOLECULE

(71) Applicant: BioArctic AB, Stockholm (SE)

(72) Inventors: Ronny Falk, Stockholm (SE); Per-Ola Freskgård, Stockholm (SE); Ken Honek, Stockholm (SE); Martin Larhammar, Stockholm (SE); Hanna Laudon, Stockholm (SE); Sebastian Meister, Stockholm (SE); Lisa Sandersjöö, Stockholm (SE)

(21) Appl. No.: 19/243,506

(22) Filed: Jun. 19, 2025

(30) Foreign Application Priority Data
Jun. 20, 2024 (EP) 24183431.6

(51) Int. Cl. C07K 16/28

(52) U.S. Cl. CPC C07K 16/2881; C07K 2317/31; C07K 2317/34; C07K 2317/565; C07K 2317/622; C07K 2317/94

(57) ABSTRACT

The present disclosure relates to a stabilized binding molecule, for example an antibody or antigen-binding fragment thereof, which binds to the protease-like domain of human transferrin receptor 1 (TfR1), and to therapeutic and diagnostic uses thereof.

### FIELD

The present disclosure relates to a stabilized binding molecule, for example an antibody or antigen-binding fragment thereof, which binds to the protease-like domain of human transferrin receptor 1 (TfR1), and to therapeutic and diagnostic uses thereof.

### BACKGROUND

Treatment modalities for brain and neurological diseases are limited, due to the impermeability of the blood vessels of the brain to most substances carried in the bloodstream (Freskgard and Urich (2017), Neuropharmacology 120:38-55; Stanimirovic et al (2018), BioDrugs 32:547-559). The small blood vessels (capillaries) of the brain, referred to collectively as the blood-brain barrier (BBB), are unique when compared to the blood vessels found in the periphery of the body. Tight apposition of BBB endothelial cells (EC) to neural cells, such as astrocytes, pericytes and neurons, induces phenotypic features that contribute to the observed impermeability. Tight junctions between ECs in the BBB limit paracellular transport, while the lack of passive pinocytotic vesicles and fenestrae limit non-specific transcellular transport. These factors combine to restrict molecular flux from the blood to the brain in general to molecules that are less than 500 Da in size and lipophilic. Thus, the otherwise promising prospect of using the large mass transfer surface area (over 20 m² from 600 km of capillaries in a human brain) of the blood stream as a delivery vehicle is made largely infeasible, except in those circumstances where a drug with the desired pharmacological properties fortuitously possesses size and lipophilicity attributes which allow it to pass through the BBB. Because of such restrictions, it has been estimated that more than 98% of all small molecule pharmaceuticals and nearly 100% of the emerging class of protein and gene therapeutics do not cross the BBB.

WO91/03259 proposes a principle for transporting a neuropharmaceutical agent across the BBB, which involves conjugating the agent to an antibody which is reactive with the transferrin receptor. According to this disclosure, binding of the conjugate to the transferrin receptor leads to active transport of the conjugate across the BBB. Later work has developed this concept further, for example as described in WO2012/075037, WO2014/033074, WO2018/011353 and WO2022/258841, all describing different formats for achieving transport of a biopharmaceutical agent across the BBB by utilizing the transferrin receptor.

There are two forms of the human transferrin receptor. Transferrin receptor 1 (TfR1) is the target for the binding molecule of the present disclosure. TfR1 is an iron transporter protein, which maintains cellular iron levels by recognizing and internalizing through specific binding of the iron carrier proteins transferrin (Tf) and ferritin (Ft) into cells through endocytosis mediated by clathrin-coated vesicles. TfR1 is expressed in numerous cells and organs, but expression levels vary and, importantly, TfR1 is expressed to a higher degree on BBB endothelial cells than on other endothelial cells, making the receptor a target for neuropharmaceutical delivery. Structurally, TfR1 is a dimeric transmembrane glycoprotein comprising the amino acid sequence SEQ ID NO:85, which has a large ectodomain (residues 89-760), an intramembrane region (residues 62-88) and a cytoplasmic domain (residues 1-61). The ectodomain in turn has three distinct domains held separate from the cell surface by a stalk region (residues 89-120). These three parts of the ectodomain are the helical domain (residues 606-760), the protease-like domain (residues 121-183, 384-605) and the apical domain (residues 184-383) (Lawrence et al (1999), Science 286:779-782).

In the context of BBB transport via the TfR1, antibodies and fragments thereof which have affinity for TfR1 have been described. By way of example, a number of TfR1 binding antibodies are disclosed in WO2014/189973, in which antibodies are grouped according to epitope specificity in classes I-IV (see e.g. FIG. 3 and the associated FIG. description on page 30 lines 11-15). Classes I-III of WO2014/189973 are denoted “apical binders” whereas the antibody of class IV is denoted a “non-apical binder”. Other TfR1 binding antibodies are disclosed in EP3088518, EP3315606 and EP3560958, however without any information about the epitope specificity of these disclosed antibodies.

Thus, most work on using TfR1 as a target for binding and BBB transport has focused on apical binders. This is thought to be because the apical domain is the structure within TfR1 that seems to provoke a strong immune response and thus to trigger antibody generation in animals when used as an immunogen. Thus, most known antibody binders against TfR1 have epitopes that are located within the apical domain. Another indication that the apical domain contains structures prone to engage with various ligands is that viruses have been described to utilize epitopes within the apical domain to enter cells (Cohen-Dvashi et al (2020), Nat Commun 11:67).

Furthermore, the detailed structure of the TfR1 and ferritin complex was recently determined (Montemiglio et al (2019), Nat Commun 10:1121), showing that the interface between TfR1 and ferritin is located within the apical domain. This suggest that TfR1 apical binders could potentially interfere with the binding of ferritin to TfR1 if used for BBB transport and in this way influence the normal function of ferritin in iron transport. Also, the binding and uptake of H-ferritin have been shown to be mediated by TfR1 (Li et al (2010), Proc Natl Acad Sci USA 107 (8): 3505-10). Thus, there are reasons to conclude that binders directed against the apical domain of TfR1, and especially binding to the binding site used by ferritin, may negatively influence the important function of ferritin in transporting iron via the binding to TfR1.

It has been reported that TfR1 apical binders can induce both acute clinical signs and decreased in circulating reticulocytes (Couch et al (2013), Sci Transl Med 5:183ra57). The TfR1 has also been described in relation to anemia and iron deficiency (Braga et al (2014), Clin Chim Acta 431:143-147). Anemia due to autoantibodies to TfR1 has also been described (Hyman et al (1984), N Engl J Med 311:214-218). Taken together, the data suggest that TfR1 binding and interfering with iron transporters such as transferrin and/or ferritin could lead to safety issues such as reduction in reticulocyte levels and anemia.

To date, the focus within the field has been to avoid interfering with one of the described TfR1 ligands, namely transferrin. This has guided the field to utilize binding sites in the apical domain of TfR1, distant from the binding site of transferrin. However, such apical binders may still interfere with the other important TfR1 ligand, ferritin, leading to interference in iron transport and function.

Single-chain Fv (scFv) domains of antibodies are recombinant proteins in which the variable regions of the heavy chain (VH) and light chain (VL) are normally linked by a flexible polypeptide linker to promote the assembly of the VL and VH domains. scFvs have the advantages of being smaller and thus more susceptible to genetic manipulation and engineering, as compared to full size antibody molecules and other, larger, types of antibody fragments. Nevertheless, the practical use of scFvs has long been limited due to low homogeneity, in turn caused by a propensity for aggregation mediated by inter-chain VH-VL interactions. Because of relatively weak interactions between its VH and VL domains, the scFv structure is in an equilibrium state between an open form, in which the two domains are dissociated, and a closed form, in which the two domains are associated through inter-domain interactions formed by surfaces matching the VL and VH together (FIG. 27). The dynamics between the open and closed forms are more prominent in an scFv than in a Fab fragment, due to a lack in an scFv of CL and CH1 regions that stabilize the structure within the Fv part. If the open state of an scFv accumulates, this could lead to inter-chain VH-VL interactions, resulting in the formation of dimers and oligomers (Arndt et al (1998) Biochemistry 37:12918-12926). The formation of these dimers or oligomers, in turn, may increase the avidity effect as they contain more than one binding site. Also, larger oligomers or aggregates likely lead to precipitation of the constructs. Due to a lack of general methods to overcome this problem, therapeutic scFv have not been in focus since their development around 1990 (Bird et al (1988) Science 242:423-426).

Against this background, the development of general methods to suppress the aggregation tendency of scFv and to stabilize the interaction between the VL and VH domains within an scFv has been necessary for these next-generation antibody formats to be successful. One such approach is the design and generation of disulfide-stabilized Fv fragments (denoted “dsFv”). This method addresses the problems of instability and aggregation frequently associated with scFv: s (see above). The VH and VL domains in dsFv are connected by an interdomain disulfide bond (Weatherill et al (2012) Protein Engineering, Design & Selection 25 (7): 321-329; Yamauchi et al (2019) Molecules 24 (14): 2620). Different locations for the engineered introduction of pairs of VL: VH interface cysteines have been described, most commonly in the context of Fv fragments (see e.g. Glockshuber et al (1990) Biochemistry 29:1362-1367; Brinkmann et al (1993) Proc. Natl. Acad. Sci. USA 92:7538-7542). To generate one such molecule, one amino acid each in the framework regions of the VH (typically at Kabat position 44) and VL (typically at Kabat position 100) domains are mutated to cysteine. These introduced cysteines then form a stable interchain disulfide bond when the VL and VH domains come into proximity with each other. The resulting dsFv (interchain disulfide bond, no linker peptide) or scdsFv (linker peptide and interchain disulfide bond) can be fused to other antibody domains and produced in various expression systems. Disulfide stabilized Fv's or scFv's may be able to solve problems that are frequently associated with Fv's or scFv's; they become very stable and, in most instances, exhibit full antigen binding activity.

Against this background, there remains a need in the field for stable antibodies and other binding molecules, which have a binding affinity for TfR1 but which do not exhibit the drawbacks and risks associated with hitherto known binding molecules.

### DISCLOSURE OF THE INVENTION

It is an object of the disclosure to address this need, by providing a TfR1 binding molecule which utilizes a different binding site on TfR1 than the naturally occurring ligands.

One such object is to provide a TfR1 binding molecule which utilizes a different binding site on TfR1 than transferrin.

Another such object is to provide a TfR1 binding molecule which utilizes a different binding site on TfR1 than ferritin.

Yet another such object is to provide a TfR1 binding molecule which utilizes a different binding site on TfR1 than HFE (homeostatic iron regulator).

A related object of the disclosure is to provide a TfR1 binding molecule which interacts with TfR1 in a way which minimizes the interference with TfR1 itself and/or its normal function.

Another object of the disclosure is to provide a TfR1 binding molecule suitable for use as a fusion partner in constructs arranged for transport through the BBB.

A related object of the disclosure is to provide a TfR1 binding molecule which exhibits an improved stability, e.g. in the form of storage stability and/or resistance against multimerization, as compared to other TfR1 binding molecules.

One or more of these objects, and other objects that are evident to the skilled person from the teachings herein, are met by the various aspects of the disclosure.

Thus, in a first aspect, the present disclosure provides a transferrin receptor 1 (TfR1) binding molecule, which
is capable of selective binding to an epitope located in the protease-like domain of TfR1 defined by amino acid residues 121-183 and 384-605 in SEQ ID NO: 85,
comprises an immunoglobulin heavy chain variable region (VH) and an immunoglobulin light chain variable region (VL), said VH and VL regions forming a VH/VL pair comprising an antigen-binding surface, in which said antigen-binding surface provides the binding molecule with the capacity to bind selectively to said epitope; and
comprises one first cysteine residue in said VH region and one second cysteine residue in said VL region, said first and second cysteine residues being arranged such that they form a disulfide bridge connecting the VH and VL regions.

Without wishing to be bound by theory, the binding to TfR1 to an epitope, or binding site, within the protease-like domain is contemplated to offer advantages in terms of avoiding the drawbacks associated with known binders to TfR1, in particular those known binders which have affinity for epitopes or binding sites located in the apical domain of TfR1.

Also without wishing to be bound by theory, the provision of the first and second cysteine residues in the VH and VL regions, respectively, and the resultant disulfide bridge between VH and VL, is contemplated to allow for the formation of a more stable VH/VL pairing. Non-limiting examples of advantages with such increased stability include an improved storage stability and an increased resistance towards multimerization.

Importantly, the increased size and avidity of dimers, or further multimers, of binding molecules comprising pairs of VH and VL regions may cause undesirable cross-linking of targets in vivo and altered pharmacodynamic properties. This is especially important when binding to the transferrin receptor to cross the blood brain barrier, because it is crucial to avoid multimerization, as this leads to down-regulation of the transferrin receptor. Such down-regulation, in turn, reduces the transport capacity over the blood brain barrier and can potentially cause safety problems for a biopharmaceutical product, due to a lower abundance of transferrin receptors on the cell surface. In addition, dimerization and further oligomerization is contemplated to pose considerable challenges with respect to the production, analysis, formulation and storage of biologics in connection with commercial or clinical applications.

hTfR1 binding proteins of the present disclosure are engineered with a disulfide bridge in order to stabilize the VL/VH or VH/VL forms. This is shown to be crucial for producing constructs that are both stable and only bind in a monomeric form to transferrin receptor. Data shows that the presence of only small amounts of dimeric forms of binding molecules leads to avidity binding to the transferrin receptor. Also, without the stabilizing disulfide, the disclosed constructs may be produced in a dimeric or oligomeric form and be unstable over time under various conditions. Thus, by introducing a disulfide bond between the VL/VH or VH/VL domains, the hTfR1 binding molecules of the disclosure are both more stably produced and in addition prevent avidity binding to the transferrin receptor.

In one embodiment, said first cysteine (in the VH region) is located at an amino acid position selected from VH position 39-49 as determined by reference to the Kabat numbering scheme. In a more specific embodiment, said first cysteine is located at an amino acid position selected from VH position 41-47, such as selected from VH position 43-45, all as determined by reference to the Kabat numbering scheme. In a yet more specific embodiment, the first cysteine is located at VH position 44 per Kabat numbering.

In one embodiment, said second cysteine (in the VL region) is located at an amino acid position selected from VL position 95-105 as determined by reference to the Kabat numbering scheme. In a more specific embodiment, said first cysteine is located at an amino acid position selected from VL position 97-103, such as selected from VL position 99-101, all as determined by reference to the Kabat numbering scheme. In a yet more specific embodiment, the first cysteine is located at VL position 100 per Kabat numbering.

In one exemplary embodiment, said first cysteine residue is located at VH position 44 and said second cysteine residue is located at VL position 100, as determined by reference to the Kabat numbering scheme.

In a specific embodiment, the epitope or binding site for the TfR1 binding molecule of the disclosure comprises the amino acid residues 150, 151, 154, 158, 159, 161, 163 and 385 in SEQ ID NO:85. In another embodiment, the epitope or binding site for the TfR1 binding molecule of the disclosure consists of the amino acid residues 150, 151, 154, 158, 159, 161, 163 and 385 in SEQ ID NO:85. In an alternative specific embodiment, the epitope or binding site for the TfR1 binding molecules comprises or consists of at least one, at least two, at least three, at least four, at least five, at least six, at least seven or all eight of the amino acid residues 150, 151, 154, 158, 159, 161, 163 and 385 in SEQ ID NO:85. As shown in the examples which follow, for example with reference to FIG. 12, this embodiment of the epitope for the binding molecules identified and disclosed herein ensures binding that does not interfere with the natural TfR1 ligands transferrin and ferritin.

As known to a person skilled in the art, an epitope (or “antigenic determinant”) is a group of amino acids or other chemical groups exposed on the surface of a molecule, frequently a protein, here hTfR1, which can generate an antigenic response and bind antibody. An epitope is a localized region on the surface of an antigen that is recognized by the immune system, specifically by antibodies. A conformational epitope is composed of neighboring amino acid residues located on an antigenic protein surface structure. Conformational epitopes bind their complementary paratopes in B-cell receptors and/or antibodies. In one embodiment of the disclosure, the epitope bound by the binding molecule is a conformational epitope.

The binding molecule comprises an immunoglobulin heavy chain variable region (VH) and an immunoglobulin light chain variable region (VL), said VH and VL regions forming a VH/VL pair comprising an antigen-binding surface, in which said antigen-binding surface provides the binding molecule with the capacity to bind selectively to said epitope. Said VH/VL pair may for example be provided in a full-length traditional antibody, or in an antibody fragment selected from the group consisting of a Fab fragment, a single chain Fab (scFab) fragment, an Fv fragment and a single chain (scFv) fragment. In a specific embodiment, the VH/VL pair forms part of an scFv. The designation of “VH/VL” as used in relation to a VH/VL pair does not limit the construct to any particular order of the VH and VL regions in the polypeptide chain, but is only used to convey that both the VH and VL regions are present, and that they are capable of pairwise association to form an Ig domain with an antigen-binding surface. As such, the term “VH/VL pair” encompasses, for example, constructs in which the VL region precedes the VH region in a single chain Fv, constructs in which the VH region precedes the VL region in a single chain Fv, and constructs in which the VH and VL regions are non-covalently associated with each other.

With respect to the antigen-binding surface of the VH/VL pair, it may suitably be composed of three complementarity-determining regions (CDRs) from said VH region and three CDRs from said VL region. In one embodiment, said CDRs comprise the following:

VHCDR1:
(SEQ ID NO: 1)
X1X2NMX3,

wherein
X1 is selected from D and A;
X2 is selected from Y and A; and
X3 is selected from D and A;

VHCDR2:
(SEQ ID NO: 2)
X4INPX5X6X7TTSX8X9X10KFKG,

wherein
X4 is selected from D and A;
X5 is selected from D, N and A;
X6 is selected from Y and A;
X7 is selected from D and A;
X8 is selected from Y and A;
X9 is selected from N and S; and
X10 is selected from E and Q;

VLCDR1:
(SEQ ID NO: 4)
KSSQSLLX11SX12NX13KNX14LA,

wherein
X11 is selected from Y and A;
X12 is selected from T and S;
X13 is selected from Q and R; and
X14 is selected from Y and A;

VLCDR2:
(SEQ ID NO: 5)
X15ASTRES

wherein
X15 is selected from W and A; and

VLCDR3:
(SEQ ID NO: 6)
QQX16X17X18X19PX20T

wherein
X16 is selected from Y and A;
X17 is selected from F and Y;
X18 is selected from I and N;
X19 is selected from Y and A; and
X20 is selected from R and Y.

In one embodiment, the CDRs further comprise:

VHCDR3:
(SEQ ID NO: 3)
GGX21SGSSX22X23HPMX24X25

wherein
X21 is selected from Y and A;
X22 is selected from Y and A;
X23 is selected from Y and A;
X24 is selected from D and A; and
X25 is selected from Y and A.

In an alternative embodiment, the CDRs further comprise:

VHCDR3:
(SEQ ID NO: 35)
SEAGNYYWYFDV

As defined herein, embodiments of the binding molecule of the first aspect of the disclosure that comprise a VH/VL pair have specific amino acid sequences in the regions determining its binding capability, such as the CDRs of the heavy and/or light chain variable domain, or indeed the entire VL and/or VH domains or regions. Non-limiting examples of such specific amino acid sequences are provided herein for the specific antibodies generated and characterized as described in Examples 1-9. It is contemplated that the specific sequence information provided for the generated antibodies enables the skilled person to define combinations and variations of these sequences within the scope of the invention, such as including the combinations and variations afforded by the variation in the general CDR sequences provided as SEQ ID NO: 1-6.

In one embodiment, said CDRs further comprise

VHCDR3:
(SEQ ID NO: 3)
GGX21SGSSX22X23HPMX24X25

wherein
X21 is selected from Y and A;
X22 is selected from Y and A;
X23 is selected from Y and A;
X24 is selected from D and A; and
X25 is selected from Y and A.

In one embodiment, said VHCDR2 is:

VHCDR2:
(SEQ ID NO: 7)
X4INPX5X6X7TTSX8NEKFKG,

wherein
X4 is selected from D and A;
X5 is selected from D and A;
X6 is selected from Y and A;
X7 is selected from D and A; and
X8 is selected from Y and A.

In one embodiment, said VLCDR1 is:

VLCDR1:
(SEQ ID NO: 8)
KSSQSLLX11STNQKNX14LA,

wherein
X11 is selected from Y and A; and
X14 is selected from Y and A.

In one embodiment, said VLCDR3 is:

VLCDR3:
(SEQ ID NO: 9)
QQX16FIX19PRT

wherein
X16 is selected from Y and A;
X19 is selected from Y and A.

In one embodiment, the amino acid sequence of said VHCDR1 is selected from the group consisting of SEQ ID NO:10 and 16-18.

In one embodiment, the amino acid sequence of said VHCDR2 is selected from the group consisting of SEQ ID NO:11, 19-23 and 34, for example selected from the group consisting of SEQ ID NO:11 and 19-23.

In one embodiment, the amino acid sequence of said VHCDR3 is selected from the group consisting of SEQ ID NO:12, 24-28 and 35, for example selected from the group consisting of SEQ ID NO:12 and 24-28.

In one embodiment, the amino acid sequence of said VLCDR1 is selected from the group consisting of SEQ ID NO:13, 29, 30 and 36, for example selected from the group consisting of SEQ ID NO:13, 29 and 30.

In one embodiment, the amino acid sequence of said VLCDR2 is selected from the group consisting of SEQ ID NO:14 and 31.

In one embodiment, the amino acid sequence of said VLCDR3 is selected from the group consisting of SEQ ID NO:15, 32, 33 and 37, for example selected from the group consisting of SEQ ID NO:15, 32 and 33.

In some embodiments, the CDR sequences can be freely combined among the options listed above. Such embodiments for example include, but are not limited to, those combinations exemplified in Example 9 for alanine substituted variants of the h26D3 embodiment of the binding molecule of the disclosure.

In a specific embodiment of a binding molecule of the disclosure comprising a VH/VL pair, the amino acid sequences of the six CDRs are the following:

VHCDR1:
(SEQ ID NO: 10)
DYNMD,
VHCDR2:
(SEQ ID NO: 11)
DINPDYDTTSYNEKFKG,
VHCDR3:
(SEQ ID NO: 12)
GGYSGSSYYHPMDY
VLCDR1:
(SEQ ID NO: 13)
KSSQSLLYSTNQKNYLA,
VLCDR2:
(SEQ ID NO: 14)
WASTRES
VLCDR3:
(SEQ ID NO: 15)
QQYFIYPRT

In another specific embodiment of a binding molecule of the disclosure comprising a VH/VL pair, the amino acid sequences of the six CDRs are the following:

VHCDR1:
(SEQ ID NO: 10)
DYNMD,
VHCDR2:
(SEQ ID NO: 21)
DINPDADTTSYNEKFKG,
VHCDR3:
(SEQ ID NO: 12)
GGYSGSSYYHPMDY
VLCDR1:
(SEQ ID NO: 13)
KSSQSLLYSTNQKNYLA,
VLCDR2:
(SEQ ID NO: 14)
WASTRES
VLCDR3:
(SEQ ID NO: 15)
QQYFIYPRT

In another specific embodiment of a binding molecule of the disclosure comprising a VH/VL pair, the amino acid sequences of the six CDRs are the following:

VHCDR1:
(SEQ ID NO: 10)
DYNMD,
VHCDR2:
(SEQ ID NO: 34)
DINPNYDTTSYSQKFKG,
VHCDR3:
(SEQ ID NO: 35)
SEAGNYYWYFDV
VLCDR1:
(SEQ ID NO: 36)
KSSQSLLYSSNRKNYLA,
VLCDR2:
(SEQ ID NO: 14)
WASTRES
VLCDR3:
(SEQ ID NO: 37)
QQYYNYPYT

In another specific embodiment of a binding molecule of the disclosure comprising a VH/VL pair, the amino acid sequences of the six CDRs are the following:

VHCDR1:
(SEQ ID NO: 38)
NYWLG,
VHCDR2:
(SEQ ID NO: 39)
DIFPGSDNTYYNEKFKG,
VHCDR3:
(SEQ ID NO: 40)
SGNFYAMDY
VLCDR1:
(SEQ ID NO: 41)
SASSSVNYMN,
VLCDR2:
(SEQ ID NO: 42)
DTSKLAS
VLCDR3:
(SEQ ID NO: 43)
FQGSGYPFT

In one embodiment, CDR sequences in an antigen-binding interface comprised in a binding molecule of the disclosure are as defined using the Kabat convention, which is well known to a person of skill in the art of antibody technology (see e.g. Kabat (1991), Sequences of Proteins of Immunological Interest, 5th edition, NIH Publication no 91-3242 from the US Department of Health and Human Services). In one embodiment of a binding molecule of the disclosure comprising a VH/VL pair, said VH region comprises or consists of an amino acid sequence selected from
(i) the group consisting of SEQ ID NO:88-103, for example the group consisting of SEQ ID NO:88-101, for example the group consisting of SEQ ID NO:88 and 94; and
(ii) a sequence having at least 80%, at least 90%, at least 92%, at least 94%, at least 95%, at least 96%, at least 97%, at least 98% or at least 99% identity to a sequence defined in (i), provided that the sequences of the CDR regions are 100% identical to those of the CDR regions in a sequence defined in (i), and provided that the sequence comprises a cysteine residue at position 44 (Kabat position 44).

In one embodiment of a binding molecule of the disclosure comprising a VH/VL pair, said VL region comprises or consists of an amino acid sequence selected from
(i) the group consisting of SEQ ID NO:105-113, for example the group consisting of SEQ ID NO:105-111; and
(ii) a sequence having at least 80%, at least 90%, at least 92%, at least 94%, at least 95%, at least 96%, at least 97%, at least 98% or at least 99% identity to a sequence defined in (i), provided that the sequences of the CDR regions are 100% identical to those of the CDR regions in a sequence defined in (i), and provided that the sequence comprises a cysteine residue at position 106 (Kabat position 100).

In a particular such embodiment, the VH region and VL region are both as defined immediately above, i.e. a VH comprising or consisting of a sequence selected from SEQ ID NO:88-103 and sequences having at least 80% sequence identity thereto, and a VL comprising or consisting of a sequence selected from SEQ ID NO: 105-113 and sequences having at least 80% sequence identity thereto, subject to the defined provisos.

In another embodiment of a binding molecule of the disclosure comprising a VH/VL pair, said VH region comprises or consists of an amino acid sequence selected from SEQ ID NO:104 and a sequence having at least 80%, at least 90%, at least 92%, at least 94%, at least 95%, at least 96%, at least 97%, at least 98% or at least 99% identity to SEQ ID NO:104, provided that the sequences of the CDR regions are 100% identical to those of the CDR regions in SEQ ID NO:104, and provided that the sequence comprises a cysteine residue at position 44 (Kabat position 44).

In another embodiment of a binding molecule of the disclosure comprising a VH/VL pair, said VL region comprises or consists of an amino acid sequence selected from SEQ ID NO:114 and a sequence having at least 80%, at least 90%, at least 92%, at least 94%, at least 95%, at least 96%, at least 97%, at least 98% or at least 99% identity to SEQ ID NO:114, provided that the sequences of the CDR regions are 100% identical to those of the CDR regions in SEQ ID NO:114, and provided that the sequence comprises a cysteine residue at position 99 (Kabat position 100).

In a particular such embodiment, the VH region and VL region are both as defined immediately above, i.e. a VH comprising or consisting of the sequence SEQ ID NO: 104 and sequences having at least 80% sequence identity thereto, and a VL comprising or consisting of the sequence SEQ ID NO:114 and sequences having at least 80% sequence identity thereto, subject to the defined provisos.

In one embodiment of a binding molecule of the disclosure comprising a VH/VL pair, said VH region comprises or consists of an amino acid sequence selected from
(i) the group consisting of SEQ ID NO:124-304, for example the group consisting of SEQ ID NO:130, 146, 163, 180, 197, 213, 230, 247, 264, 279 and 296, for example the group consisting of SEQ ID NO:130, 146, 163, 180, 197 and 213, for example the group consisting of SEQ ID NO:130 and 146; and
(ii) a sequence having at least 80%, at least 90%, at least 92%, at least 94%, at least 95%, at least 96%, at least 97%, at least 98% or at least 99% identity to a sequence defined in (i), provided that the sequences of the CDR regions are 100% identical to those of the CDR regions in a sequence defined in (i), and provided that the sequence comprises a cysteine residue at position 44 (Kabat position 44). In one such embodiment of a binding molecule of the disclosure comprising a VH/VL pair, said VL region comprises or consists of an amino acid sequence selected from
(i) the group consisting of SEQ ID NO:105-113, for example the group consisting of SEQ ID NO:105-111; and
(ii) a sequence having at least 80%, at least 90%, at least 92%, at least 94%, at least 95%, at least 96%, at least 97%, at least 98% or at least 99% identity to a sequence defined in (i), provided that the sequences of the CDR regions are 100% identical to those of the CDR regions in a sequence defined in (i), and provided that the sequence comprises a cysteine residue at position 106 (Kabat position 100).

In a particular such embodiment, the VH region and VL region are both as defined immediately above, i.e. a VH comprising or consisting of a sequence selected from SEQ ID NO:124-304 and sequences having at least 80% sequence identity thereto, and a VL comprising or consisting of a sequence selected from SEQ ID NO: 105-113 and sequences having at least 80% sequence identity thereto, subject to the defined provisos.

In one embodiment of a binding molecule of the disclosure comprising a VH/VL pair, said VH region comprises SEQ ID NO:88 and said VL region comprises a sequence selected from SEQ ID NO:105-111.

In one embodiment of a binding molecule of the disclosure comprising a VH/VL pair, said VH region comprises a sequence selected from SEQ ID NO:88-101 and said VL region comprises SEQ ID NO:105.

In one embodiment of a binding molecule of the disclosure comprising a VH/VL pair, said VH region comprises SEQ ID NO:88 and said VL region comprises SEQ ID NO:105.

In one embodiment of a binding molecule of the disclosure comprising a VH/VL pair, said VH region comprises SEQ ID NO:94 and said VL region comprises SEQ ID NO:105.

In one embodiment of a binding molecule of the disclosure comprising a VH/VL pair, said VH region comprises SEQ ID NO:130 and said VL region comprises a sequence selected from SEQ ID NO:105-111.

In one embodiment of a binding molecule of the disclosure comprising a VH/VL pair, said VH region comprises SEQ ID NO:146 and said VL region comprises a sequence selected from SEQ ID NO:105-111.

In one embodiment of a binding molecule of the disclosure comprising a VH/VL pair, said VH region comprises a sequence selected from SEQ ID NO:124-304 and said VL region comprises SEQ ID NO:105.

In one embodiment of a binding molecule of the disclosure comprising a VH/VL pair, said VH region comprises SEQ ID NO:130 and said VL region comprises SEQ ID NO:105.

In one embodiment of a binding molecule of the disclosure comprising a VH/VL pair, said VH region comprises SEQ ID NO:146 and said VL region comprises SEQ ID NO:105.

In certain embodiments, the VH and VL sequences, when present in the binding molecule, are selected from any one of the listed sequences and sequences having at least 80%, at least 90%, at least 92%, at least 94%, at least 95%, at least 96%, at least 97%, at least 98%, at least 99% or at least 100% identity thereto.

In one embodiment of a binding molecule of the disclosure comprising a VH/VL pair, said VH/VL pair forms part of an antibody construct.

In one such embodiment, said antibody construct has more than one binding specificity. It may for example be bispecific or trispecific, or have more than three binding specificities. In a particular embodiment, the binding molecule is bispecific, for example a bispecific antibody construct.

In one embodiment of a binding molecule of the disclosure comprising a VH/VL pair, the VH/VL pair is present in an antibody fragment selected from the group consisting of a Fab fragment, a single chain Fab (scFab) fragment, an Fv fragment and a single chain (scFv) fragment. In a particular embodiment of such a binding molecule, said antibody fragment is an scFv.

In an important embodiment of the binding molecule disclosed herein, it further comprises an antibody or an antigen binding fragment thereof, in addition to the VH/VL pair providing selective binding to hTfR1. In one such embodiment, this additional antibody or fragment thereof is capable of selective binding to a target present in the brain of a mammal. In some embodiments, said target is selected from the group consisting of amyloid-β peptide or derivatives or fragments thereof, alpha-synuclein or derivatives or fragments thereof, TAR DNA-binding protein 43 (TDP-43) or derivatives or fragments thereof, triggering receptor expressed on myeloid cells 2 (TREM2), beta-secretase 1 (BACE1), superoxide dismutase (SOD), huntingtin, transthyretin, P-secretase 1, epidermal growth factor, epidermal growth factor receptor 2, Tau, phosphorylated Tau or fragments thereof, apolipoprotein E4, CD20, prion protein, leucine rich repeat kinase 2, parkin, presenilin 2, gamma secretase, death receptor 6, amyloid-β precursor protein, p75 neurotrophin receptor, neuregulin and caspase 6. In a more specific embodiment, said target is selected from the group consisting of amyloid-β peptide or derivatives or fragments thereof, alpha-synuclein or derivatives or fragments thereof, TAR DNA-binding protein 43 (TDP-43) or derivatives or fragments thereof, triggering receptor expressed on myeloid cells 2 (TREM2), Tau, phosphorylated Tau or fragments thereof and apolipoprotein E4. In an even more specific embodiment, said target is selected from the group consisting of amyloid-β peptide or derivatives or fragments thereof, alpha-synuclein or derivatives or fragments thereof and TAR DNA-binding protein 43 (TDP-43) or derivatives or fragments thereof.

In one embodiment, said antibody or antigen binding fragment thereof capable of selective binding to a target present in the brain of a mammal is an anti-Aβ antibody, for example an antibody selected from the group consisting of lecanemab, gantenerumab, aducanumab, donanemab, PBD-C06 and KHK6640.

In another embodiment, said antibody or antigen binding fragment thereof capable of selective binding to a target present in the brain of a mammal is an anti-alpha-synuclein antibody, for example an antibody selected from the group consisting of prasinezumab, UCB7853, Lu AF82422, TAK-341 and BAN0805.

### Affinity for a Target

As used herein, the terms “specific binding to X”, “selective binding to X” and “affinity for X”, wherein X is a target (e.g. an antigen or an epitope, such as the TfR1 bound by the VH/VL pair of the binding molecule as defined above), refer to a property of a binding molecule, such as a property of an antibody or antigen-binding fragment thereof or of a bi- or multispecific construct incorporating such an antibody or antigen-binding fragment thereof, which may be tested for example by ELISA, by surface plasmon resonance (SPR) or by bio-layer interferometry (BLI). The skilled person is aware of these methods and others.

For example, the binding affinity for a target, antigen or epitope X may be tested in an experiment in which a binding molecule to be tested is captured on ELISA plates coated with X or a molecule comprising the epitope X, and a biotinylated detector antibody is added, followed by streptavidin-conjugated horse radish peroxidase (HRP). Alternatively, said detector antibody may be directly conjugated with HRP. Tetramethylbenzidine (TMB) substrate is added and the absorbance at 450 nm is measured using an ELISA multi-well plate reader. The skilled person may then interpret the results obtained by such experiments to establish at least a qualitative measure of the binding affinity for X of the binding molecule. If a quantitative measure is desired, for example to determine the EC50 value (the half maximal effective concentration) for the interaction, ELISA may also be used. The response of the binding molecule against a dilution series of X may be measured using ELISA as described above. The skilled person may then interpret the results obtained by such experiments and EC50 values may be calculated from the results, using for example GraphPad Prism v.9 and non-linear regression.

As used herein, the term “EC50” refers to the half maximal effective concentration of binding molecule which induces a response halfway between the baseline and maximum after a specified exposure time.

Additionally or alternatively, inhibition ELISA may be used to obtain a quantitative measure of interaction by determination of the “IC50” (the half maximal inhibitory concentration). In an inhibition ELISA, the concentration of target X in a fluid sample is measured by detecting interference in an expected signal output. In principle, a known target or epitope-bearing substance is used to coat a multi-well plate. In parallel, a binding molecule with putative affinity for X is added and incubated with a solution containing target at varied concentrations. Following standard blocking and washing steps, samples containing the mixture of said binding molecule and the target are added to the well. Labeled detection antibody with affinity for the binding molecule is then applied for detection using relevant substrates (for example TMB). In principle, if there is a high concentration of target in the fluid sample, a significant reduction in signal output will be observed. In contrast, if there is very little target in the fluid sample, there will be very little reduction in the expected signal output. The skilled person appreciates that the signal output is also dependent on the affinity of the binding molecule for said target.

As used herein, the term “IC50” refers to the half maximal inhibitory concentration of a binding molecule which induces a response halfway between the baseline and maximum inhibition after a specified exposure time. Herein, a lower IC50 value indicates that a lower concentration of target is required to interfere with the binding of the detection antibody to the known target coated on the plate, as compared to a higher IC50 value. Thus, a lower IC50 value typically corresponds to a higher affinity.

The binding affinity of a binding molecule may also be tested by surface plasmon resonance (SPR). For example, the affinity may be tested in an experiment in which target or epitope X is immobilized on a sensor chip of the instrument, and the sample containing the binding molecule to be tested is passed over the chip. Alternatively, the binding molecule to be tested may be immobilized on a sensor chip of the instrument, and a sample containing X is passed over the chip. The skilled person may then interpret the results obtained by such experiments to establish at least a qualitative measure of the binding affinity for X of the binding molecule. If a quantitative measure is desired, for example to determine a K_{D} value for the interaction, SPR may also be used. Binding values may for example be defined in a Biacore (Cytiva) or ProteOn XPR 36 (Bio-Rad) instrument. The target or epitope is suitably immobilized on a sensor chip of the instrument, and samples of the binding molecule whose affinity is to be determined are prepared by serial dilution and injected. K_{D} values may then be calculated from the results using for example the 1:1 Langmuir binding model of the Biacore Insight Evaluation Software 2.0 or other suitable software, typically provided by the instrument manufacturer.

The binding affinity may also be measured by bio-layer interferometry (BLI), a label-free technology for measuring biomolecular interactions within the interactome. It is an optical analytical technique that analyzes the interference pattern of white light reflected from two surfaces: a layer of immobilized protein on the biosensor tip, and an internal reference layer. The binding between a ligand (target or epitope X) immobilized on the biosensor tip surface and an analyte (such as a binding molecule with a putative affinity for X) in solution produces an increase in optical thickness at the biosensor tip resulting in a wavelength shift, A, which is a direct measure of the change in thickness of the biological layer. Interactions are measured in real time, providing the ability to monitor binding specificity, rates of association and dissociation, or concentration, with precision and accuracy.

The skilled person is aware of the above mentioned and other methods for measuring the affinity of a binding molecule for a target or epitope X, either qualitatively or quantitatively or both.

### Stability of a Binding Molecule of the Disclosure

The introduction of cysteine residues and the resulting formation of a disulfide bridge in the binding molecule of the disclosure is contemplated to increase the stability of the binding molecule. As realized by a person of skill in the art from the context herein, such increased stability may for example be measured as an increase in the monomeric content of the binding molecule in a sample after storage, compared to the monomeric content of a binding molecule having an identical sequence except for the cysteine residues. Evaluating monomeric content may for example be done using size exclusion liquid chromatography (SEC) after simulated stress conditions and/or long-term storage. This will provide measures of the monomer content and presence of aggregates. In one embodiment, the binding molecule of the disclosure is defined as stable if it exhibits a monomer content of 90% or more after storage for two weeks at 40° C. as determined by SEC. In another embodiment, the binding molecule of the disclosure is defined as stable if it exhibits a monomer content of 95% or more after storage for two weeks at 40° C. as determined by SEC. In yet another embodiment, the binding molecule of the disclosure is defined as stable if it exhibits a monomer content of 98% or more after storage for two weeks at 40° C. as determined by SEC.

### Pharmaceutical Compositions

In a second aspect, the disclosure provides a pharmaceutical composition comprising a binding molecule as described herein and at least one pharmaceutically acceptable excipient or carrier.

Techniques for formulating polypeptides such as antibodies and their derivatives for human therapeutic use are well known in the art and are reviewed, for example, in Wang et al (2007), J Pharm Sci, 96:1-26, the contents of which are incorporated herein in their entirety.

Pharmaceutically acceptable excipients that may be used to formulate the compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene block polymers, polyethylene glycol and wool fat.

In certain embodiments, the pharmaceutical compositions are formulated for administration to a subject via any suitable route of administration including but not limited to intramuscular, intravenous, intradermal, intraperitoneal injection, subcutaneous, epidural, nasal, oral, rectal, topical, inhalational, buccal (e.g., sublingual), and transdermal administration. In preferred embodiments, the composition is formulated for intravenous or subcutaneous administration.

### Methods of Prevention, Treatment, Diagnosis, Prognosis and Detection

The binding molecule according to the present disclosure may be useful as a therapeutic, prophylactic, diagnostic and/or prognostic agent.

Hence, in a further aspect of the disclosure, there is provided a binding molecule according to the first aspect, or a pharmaceutical composition according to the second aspect, for use as a medicament.

In yet another aspect of the disclosure, there is provided a binding molecule according to the first aspect, or a pharmaceutical composition according to the second aspect, for use as a diagnostic agent.

In yet another aspect of the disclosure, there is provided a binding molecule according to the first aspect, or a pharmaceutical composition according to the second aspect, for use as a prognostic agent.

Also provided are methods of preventing, treating or diagnosing disease or assessing disease prognosis, wherein a binding molecule as disclosed herein is administered to a subject in need thereof, typically a human subject.

Also provided is the use of the disclosed binding molecule for the manufacture of compositions (such as medicaments) for use in the prevention, treatment, diagnosis and/or prognosis of any one of the listed diseases.

Thus, in one embodiment, the binding molecule, or pharmaceutical composition comprising it, is useful in the treatment, prevention, diagnosis and/or prognosis of a neurodegenerative disorder, for example a disorder selected from Alzheimer's disease and other disorders associated with Aβ protein aggregation, traumatic brain injury (TBI), Lewy body dementia (LBD), Down's syndrome (DS), amyotrophic lateral sclerosis (ALS), frontotemporal dementia, tauopathy, systemic amyloidosis, atherosclerosis, Parkinson's disease (PD), Parkinson's disease dementia (PDD), the Lewy body variant of Alzheimer's disease, multiple system atrophy, psychosis, schizophrenia, Creutzfeldt-Jakob disease, Huntington's disease, and familial amyloid neuropathy.

In a more specific embodiment, said disorder is selected from Alzheimer's disease and other disorders associated with Aβ protein aggregation, Lewy body dementia (LBD), Down's syndrome (DS), amyotrophic lateral sclerosis (ALS), frontotemporal dementia, tauopathy, Parkinson's disease (PD), Parkinson's disease dementia (PDD) and the Lewy body variant of Alzheimer's disease.

In an even more specific embodiment, said disorder is selected from Alzheimer's disease and other disorders associated with Aβ protein aggregation, Lewy body dementia (LBD), amyotrophic lateral sclerosis (ALS) and Parkinson's disease (PD), in particular Alzheimer's disease.

In an alternative embodiment, the binding molecule, or pharmaceutical composition comprising it, is useful in the treatment, prevention, diagnosis and/or prognosis of another disorder, for example a disorder selected from brain cancer, multiple sclerosis and lysosomal storage diseases.

In another aspect, there is provided a method of treatment, prevention, diagnosis and/or prognosis of a disorder as listed above, said method comprising administering to said mammal an amount, such as a therapeutically effective amount, of a binding molecule, or pharmaceutical composition comprising it.

### INCORPORATION BY REFERENCE

Various publications are cited in the present application, each of which is incorporated by reference herein in its entirety.

### BRIEF DESCRIPTION OF THE FIGURES

FIG. 1 shows the results of a binding screen of the indicated IgG antibodies from the immunization described in Example 1 towards human (hTfR1), cyno (cTfR1) and mouse (mTfR1) TfR1 in crude hybridoma supernatants by biolayer interferometry (BLI).

FIG. 2 shows the result of the BLI binding analysis described in Example 2 for the indicated Fab fragments of mouse antibodies 24B4, 26D3 and 37D10 as well as for a Fab fragment of control antibody 8D3.

FIG. 3 shows mapping of antibody binding epitopes to the protease-like domain of hTfR1 as described in Example 2, by selective antibody binding to ELISA plates coated with either human, mouse or one of three different chimeric human/mouse TfR1 receptors. Antibodies 24B4, 26D3 and 37D10 bind to hTfR1 as seen in FIG. A but not to mTfR1 as seen in FIG. B. In addition, 24B4, 26D3 and 37D10 also bind to h/m protease like domain chimera as seen in FIG. D, but not to any of the plates coated with the other chimeric receptors as seen in FIG. C and FIG. E.

FIG. 4 illustrates the epitope binning assay described in Example 2, with the following main four steps: Step 1-immobilization of bio-TfR1 on sensor chip; Step 2 wash of non-binding material; Step 3-binding of competing binder to TfR1; Step 4-association of binders to the TfR1: binder complex formed in Step 3. The data in Step 4 determines whether the two investigated binders compete in binding to hTfR1.

FIG. 5 shows the result of carrying out the epitope binding assay as described in Example 2, showing the degree of competition between antibodies for simultaneous binding to hTfR1. FIG. 5A shows binding of antibody 26D3 to preformed complexes of hTfR1 and either of the indicated antibodies. FIG. 5B shows binding of antibody 24B4 to preformed complexes of hTfR1 and either of the indicated antibodies. FIG. 5C shows binding of control antibody 15G11-1 to preformed complexes of hTfR1 and either of the indicated antibodies. Binding responses for all antibodies are normalized to the binding response measured to free hTfR1 (no competing antibody).

FIG. 6 shows binding by the indicated binders to hTfR1 on the surface of cells, studied as described in Example 2. The Y axes of both diagrams show the mean fluorescence intensity when staining cells with whole antibodies as in FIG. 6A and Fab fragments as in FIG. 6B of the indicated binders. No background staining is detected with the negative isotype control IgG as in FIG. 6A or the non-related Fab fragment, Ly128 as in FIG. 6A.

FIG. 7 shows the result of competition analysis of indicated binders with ferritin and transferrin as described in Example 3. The diagrams show in FIG. 7A MFI of the indicated binders binding to TfR1 expressed on THP-1 cell surfaces, in FIG. 7B MFI of ferritin on cell surface when exposed to the indicated binders, with the positive control antibody MA-712 competing with ferritin, and in FIG. 7C MFI of transferrin on cell surfaces when exposed to the indicated binders.

FIG. 8 is a collection of sensorgrams showing the result of SPR analysis of original 26D3 and 26D3 humanized as described in Example 4 (h26D3) in Fab formats when binding to hTfR1 and cTfR1 as indicated.

FIG. 9 shows the result of BLI and ELISA binding studies carried out on mouse and humanized versions of 26D3 in an scFv format as described in Example 4. FIG. 9A shows sensorgrams obtained by BLI measurement of binding of the indicated constructs to hTfR1. FIG. 9B shows binding responses from ELISA measurement of binding of the indicated constructs to coated TfR1.

FIG. 10 are depictions of the x-ray structure of the complex of 26D3-Fab and hTfR1, determined as described in Example 5. The chain names as used in the coordinate files are indicated. FIG. 10A shows refined structure showing overall folds of three independent complexes in the asymmetric unit. FIG. 10B shows example of electron density (2m|Fo|˜D|Fc|) contoured at the 1 σ level. The protein chains are drawn in cartoon representation while sugar moieties are shown in stick representation.

FIG. 11 is a ribbon representation of the h26D3-Fab human TfR1 complex determined with x-ray crystallography as described in Example 5. h26D3-Fab is depicted in dark gray and hTfR1 in white. The binding interface (epitope/paratope) is encircled.

FIG. 12 is a surface area representation of hTfR1 with the binding sites for the natural ligands ferritin and transferrin indicated, as well as the epitope for the binder 26D3 of the present disclosure. The different binding sites and epitope are depicted with a circle around each specific site.

FIG. 13 illustrates the work on generating and characterizing an hTfR1-KI mouse model as described in Example 6. FIG. 13A is a schematic illustration of the transgenic hTfR1-KI mouse construct. The extracellular domain of human TFRC was inserted in the murine Tfrc gene by homologous recombination. FIG. 13B is a quantitative reverse transcription PCR (RT-qPCR) analysis of human TFRC gene expression in brain (N=3/genotype). hTfR1-KI mice (grey circles) express human TFRC and mouse Tfrc in total brain homogenate, WT littermates only express mouse Tfrc (white). FIG. 13C is a quantitative reverse transcription PCR (RT-qPCR) analysis of mouse Tfrc gene expression in brain (N=3/genotype). hTfR1-KI mice (grey circles) express human TFRC and mouse Tfrc in total brain homogenate, WT littermates only express mouse Tfrc (white). FIG. 13D is a western blot analysis for hTfR1, total TfR1, and B-actin in brain. hTfR1-KI animals at 6-8 months (N=5) and 15 months (N=4) express comparable levels of hTfR1 protein. Total TfR1 levels are comparable between hTfR1-KI transgenic and WT littermates (N=3).

FIG. 14 shows the results of in vivo brain and plasma exposure analysis of various indicated hTfR1 binding molecules in hTfR1-KI transgenic mice as described in Example 7. FIG. 14A shows brain exposure 24 h after i.v. administration of the indicated hTfR1 binders. FIG. 14B shows plasma exposure 24 h after i.v. administration of the indicated hTfR1 binders. FIG. 14C shows Brain: Plasma ratio 24 h after i.v. administration of the indicated hTfR1 binders. The negative control is denoted “158”, and the positive control “15G11-1”.

FIG. 15 shows the results of in vivo brain exposure analysis of various indicated hTfR1 binding molecules in hTfR1-KI mice by immunohistochemistry as described in Example 8. Cortical brain capillary staining observed for several binding molecules, including h26D3. Reference hTfR1-binder “15G11-1” and non-TfR1 binder “158” were used as positive and negative control, respectively.

FIG. 16 shows BLI sensorgrams for the indicated alanine variants of h26D3 as described in Example 9. Each variant showed a different kinetic profile, illustrating the possibility to generate variants with different affinities against human TfR1 with specific mutations in the CDR regions of the heavy or light chain.

FIG. 17 shows representative SPR sensorgrams of the interaction between the indicated alanine variants of h26D3 with hTfR1 and cTfR1, measured as described in Example 9.

FIG. 18 shows the results of indirect ELISA analysis of the binding of the indicated alanine variants of h26D3 with hTfR1 and cTfR1, measured as described in Example 9.

FIG. 19 shows SPR sensorgrams of the interaction between the indicated alanine variants of h26D3, studied as scFv building blocks within a bispecific protein format as described in Example 9.

FIG. 20 shows chromatograms from preparative SEC of h26D3-HC6_DS as shown in FIG. 20A and h26D3-HC6 as shown in FIG. 20B, carried out as described in Example 10.

FIG. 21 shows chromatograms from analytical SEC of the indicated scFv proteins after formulation and short-term storage at −80° C., as described in Example 11.

FIG. 22 shows chromatograms from analytical SEC analysis of the indicated scFv proteins kept at −80° C. (TO) and then at 40° C. for 1, 2 and 4 weeks as indicated, carried out as described in Example 12.

FIG. 23 is a series of bar diagrams showing the percentage of monomeric scFv, as measured by analytical SEC, in samples of the respective indicated scFv molecule subjected to the thermal stability evaluation described in Example 12.

FIG. 24 shows chromatograms from analytical SEC analysis of the indicated scFv proteins kept at −80° C. (TO) and then at 40° C. for 1, 2 and 4 weeks as indicated, carried out as described in Example 12. The asterisk (*) in FIG. 24C highlights a shift in retention time for h26D3-LC1_DS which occurred due to drift in the chromatography equipment. The shift was also seen for a standard size control (not shown) injected on the same column, and is unrelated to the analyzed sample.

FIG. 25 demonstrates the results of the ELISA experiment described in Example 13, showing in FIG. 25A hTfR1 binding of a stabilized binding molecule of the disclosure after 48 h incubation in mouse serum at 37° C. and 4° C. in three separate experiments, in FIG. 25B binding curves obtained from the binding molecule incubated in serum, in comparison to incubation in PBS, and in FIG. 25C the ratio of binding activity at 37° C. to the binding activity at 4° C. in serum or PBS as indicated.

FIG. 26 shows representative SPR sensorgrams of the interaction between the indicated variants of h26D3 with hTfR1, measured as described in Example 15.

FIG. 27 shows a schematic model of the open and closed states of scFv molecules in “VH first” (top) and “VL first” (bottom) configurations, and how the formation of dimeric and further multimeric forms is prevented by the introduction of a disulfide bridge between the VH and VL domains. The disulfide bridge stabilizes the scFv in the closed state (left side), preventing the open monomeric state (middle). Were the scFv protein to transform into the open monomeric state it can proceed to form of dimer, trimer, and larger aggregates mediated by inter-chain VH-VL interactions (right side). For simplicity, only the monomeric and dimeric forms of the scFv are shown.

FIG. 28 shows stacked chromatograms from analytical SEC of the indicated scFv samples collected as TO or after temperature hold at 40° C. for 1, 2 or 4 weeks respectively, as described in Example 17. The relative absorbance mAU (220 nm) is shown on the X-axis of respective graph (FIGs. A-F).

FIG. 29 shows representative SPR sensorgrams of the interaction between the indicated scFv variants and hTfR1, as described in Example 19.

FIG. 30 shows representative SPR sensorgrams of the interaction between the indicated scFv variants and cTfR1, as described in Example 19.

FIG. 31 shows the results from screening and competition ELISA with serum from 107 donors against h26D3-HC6 scFv variants, as described in Example 20: FIG. 31A shows PE-ADA screening ELISA with 21 serum samples against TfR1 binding scFv variants, as indicated; FIG. 31B shows screening ELISA with serum from 107 donors against six selected variants show a reduced response frequency for all six variants compared to “h26D3-HC6_DS, VL-first”; FIG. 31C shows ELISA with 107 serum samples against h26D3-HC6_DS, VL-first, with competition (HC6 competition) och without competition (HC6 screen) (dotted line represents the assay cut-off point in the competition setting); FIG. 31D shows that among the serum samples from 107 donors, a majority of the samples render a response level above the assay cut-off point (horisontal line) in the ELISA setting without competition.

### EXAMPLES

While the invention has been described with reference to various exemplary aspects and embodiments, it will be understood by those skilled in the art that various changes may be made and equivalents may be substituted for elements thereof without departing from the scope of the invention. In addition, many modifications may be made to adapt a particular situation or molecule to the teachings of the invention without departing from the essential scope thereof. Therefore, it is intended that the invention is not limited to any particular embodiment, but that the invention will include all embodiments falling within the scope of the appended claims.

The invention will be further illustrated by the following non-limiting Examples. They are offered for illustrative purposes only and are not intended to limit the invention in any manner. Those of skill in the art will readily recognize a variety of non-critical parameters which can be changed or modified to yield essentially the same results. Efforts have been made to ensure accuracy with respect to numbers used (e.g. amounts, temperatures, etc.), but some experimental error and deviations may be present. Unless otherwise indicated, the practice of the invention employs conventional methods of protein chemistry, biochemistry, recombinant DNA techniques and pharmacology, within the skill of the art. Such techniques are explained fully in the existing literature. Additionally, it will be apparent to one of skill in the art that the methods for protein engineering applied herein can also be applied to other constructs described herein and contemplated by the present inventors to fall within the scope of the disclosure.

### Example 1

### Identification of Binders of Human TfR1 by Immunization and Screening

### Immunization and Hybridoma Screening

To identify monoclonal antibodies that bind human transferrin receptor 1 (hTfR1), four 6-10 weeks old Balb/c or C57BL/6 mice were immunized subcutaneously with immunogen together with adjuvant. The hTfR1 immunogen was designed to contain the ectodomain of the human TfR1 protein, N-terminally fused to a T-cell epitope from tetanus toxin, P2 (Kovacs-Nolan and Mine (2006), Biochim Biophys Acta 1760:1884-1893) via a GSS linker, and an N-terminal 10x histidine tag (His10-P2-hTfR1; SEQ ID NO:71). Following gene construction, recombinant His10-P2-hTfR1 protein was generated by transient transfection in Hek293 cells using the Expi293™ Expression system (Gibco), purified on a nickel column (HisTrap FF, cat. no. 17-5255-01, GE Healthcare), buffer exchanged to PBS and concentrated to 1 mg/ml. Expressed TfR1 immunogen was aliquoted and stored at −80° C. until use. Quil-A adjuvant (vac-quil, InvivoGen) was used for all immunizations except for the final booster injection in which no adjuvant was included. For use, Quil-A was resuspended in ddH₂O at a concentration of 1 mg/ml, sterile filtered and aliquoted in 0.1 ml aliquots stored at −80° C. Quil-A was administered at a dose of 10 μg/mouse.

Animals were immunized every month with the recombinantly produced immunogen, His₁₀-P2-hTfR1, mixed and co-administered with Quil-A. Three weeks after each immunization, blood samples were collected, and the plasma was analyzed for presence of antibodies reactive towards recombinantly produced human TfR1 and mouse TfR1. Titers were considered high enough when the ELISA response at 1/100,000 dilution exceeded the average of the blanks (i.e. background) plus 3 standard deviations of the blanks. The four mice used in this study received between 4 and 6 immunizations each.

Three days before fusion, the final intraperitoneal booster injection was given to the mice in absence of adjuvant. At sacrifice, mice were anesthetized with isoflurane. Intact spleens were collected by opening the abdominal cavity and dissected. Briefly, a single cell suspension of the spleen from an immunized mouse was prepared and mixed with Sp2/0 cells at a 3:1 ratio. The cells were fused using PEG and the cells were added to a bottle of ClonaCell™-HY Medium D (STEMCELL Technologies). 60-70 μl per well was then dispensed into 96-well plates. After 6-7 days, 150 μl HAT-medium was added to each well in the semi solid 96-well plates. The day after, 120 μl of supernatant was discarded from each well and 100 μl fresh HAT-medium was added. The next day, 100 μl of the supernatant of each well was taken and transferred to a storage plate and tested for presence of antibodies against mouse TfR1 using indirect ELISA on nickel-coated plates according to the protocol below. A repeated screen of the hybridoma plates was performed by adding 120 μl HAT-medium on day 12 and by 3 days later transferring 25 μl supernatant to ELISA plates to screen for reactivity against mouse TfR1 (both screens referred to as “primary screen”). Clones that were positive towards mouse TfR1 with OD>0.2 were transferred to 24-well plates, cultured for at least 3 days, and subjected to a secondary screen for reactivity towards murine, human and cynomolgus TfR1 in solution using biolayer interferometry (BLI) (referred to as “secondary screen”). Whereas binding of both hTfR1 and cynomolgus TfR1 was indicated, only very weak or no binding was detected for mTfR1 in the secondary screen. Supernatants from 24-well plates were also screened for binding towards His-tagged hTfR1 as well as lack of binding towards His-tagged amyloid-β precursor protein (APP; negative control) using both direct coated TfR1 plates and nickel-coated plates as described below. Binding towards cynomolgus TfR1 (cTfR1) was also analyzed using direct TfR1 coat. Notably, ELISA responses (OD450 values) were very low for mTfR1 compared to hTfR1 and cTfR1, indicating weaker binding to mTfR1 compared to the binding to hTfR1 and cTfR1 for all positive clones.

Selected clones were diluted using limiting dilution assays (LDA) to reach monoclonality. Reactivity against mouse TfR1 and human TfR1 were re-tested by ELISA on monoclonal cultures following LDA and expansion.

### Indirect ELISA Screening

ELISA assays were performed according to standard ELISA protocols in order to screen plasma samples for reactivity towards the target antigens after immunizations, or to identify hybridoma clones producing antibodies with reactivity against the TfR1 target protein. Briefly, 96-well half area plates (Corning) were coated with 1 μg/ml His₁₀-mTfR1 (SEQ ID NO:72) or His₁₀-hTfR1 (SEQ ID NO:73). His₁₀-mTfR1 and His₁₀-hTfR1 were recombinantly produced and purified using the procedure described above for the His₁₀-P2-hTfR1 immunogen. The plates were blocked with 150 μl/well of protein free blocking solution (Pierce) for 1 h at room temperature with shaking (600-900 rpm). The plates were washed four times with PBS containing 0.1% TWEEN®-20 and Kathon™. Plasma samples serially diluted from a starting dilution of 1/450 or hybridoma supernatants diluted 1/2 were added to the plates (50 μl/well; dilution buffer: PBS with 0.1% BSA and 0.05% TWEEN®-20) and incubated for 2 h at room temperature and then the plates were washed four times. Detection antibody (HRP-conjugated anti-mouse IgG, Southern Biotech, cat. no. 1030-05, diluted 1/5000 in dilution buffer) was added at 50 μl/well, and the plates were incubated for 1 h at room temperature. After another wash (as above), 50 μl/well TMB substrate (K-Blue® Aqueous, Neogen) was added, and the reaction was stopped after 10-15 min with 50 μl/well of 0.5 M H₂SO₄. The optical density at 450 nm was read using a plate reader (Tecan). The endpoint titers were defined as the dilution above the average of the blank wells (background) plus 3 standard deviations of the blank wells.

The primary screen of hybridoma clones producing antibodies with reactivity against the target protein was performed using nickel-coated ELISA plates. Briefly, 96-well Ni-coated plates (PIERCE) supplied pre-blocked with BSA were incubated with 3 μg/ml (100 μl) His₁₀-mTfR1 without shake overnight at 4° C. The plates were washed four times with PBS containing 0.1% TWEEN®-20 and Kathon™. Hybridoma supernatants diluted 1/4 were added to the plates (dilution buffer: PBS with 0.1% BSA and 0.05% TWEEN®-20) and incubated for 2 h at room temperature and then the plates were washed four times. Detection antibody (HRP-conjugated anti-mouse IgG, Southern Biotech, cat. no. 1030-05, diluted 1/5000 in dilution buffer) was added at 100 μl/well, and the plates were incubated for 1 h at room temperature. After another wash (as above), 100 μl/well of K-Blue® Aqueous substrate (Neogen) was added, and the reaction was stopped after 10-15 min with 100 μl/well of 0.5 M H₂SO₄. The optical density at 450 nm was read using an ELISA plate reader (Tecan).

Examples of clones considered to be positive in binding mouse TfR1 and human TfR1 are shown in Table 1. These clones were also confirmed to bind both His-tagged hTfR and cTfR by ELISA, and to lack binding to His-tagged APP (negative control). Selected clones were further characterized in various assays.

TABLE 1
Examples of identified clones from hybridoma screening

| | mTfR1 specificity | hTfR | cynoTfR | mTfR1 | Isotype |
|---|---|---|---|---|---|
| | by indirect ELISA | binding | binding by | binding by | (from |
| Clone | (Ni capture) | by BLI | BL | BL | sequencing) |
| 24B4 | OD > 0.2 | Yes | Yes | No/weak | IgG2a/K |
| 26D3 | OD > 0.2 | Yes | Yes | No/weak | IgG2a/K |
| 37D10 | OD > 0.2 | Yes | Yes | No/weak | IgG2a/K |

### Biolayer Interferometry Measurements

Selected clones were investigated using biolayer interferometry (BLI) on an Octet instrument (Octet Red384, ForteBio). In the setup used, the adopted method involves capture of IgG from the respective clone on the individual sensor tips to allow for detection of antibodies that bind to target in solution. In addition to providing a measure of binding, BLI measurements provide more details about the overall binding properties, because they include estimates of the on-rate and off-rate.

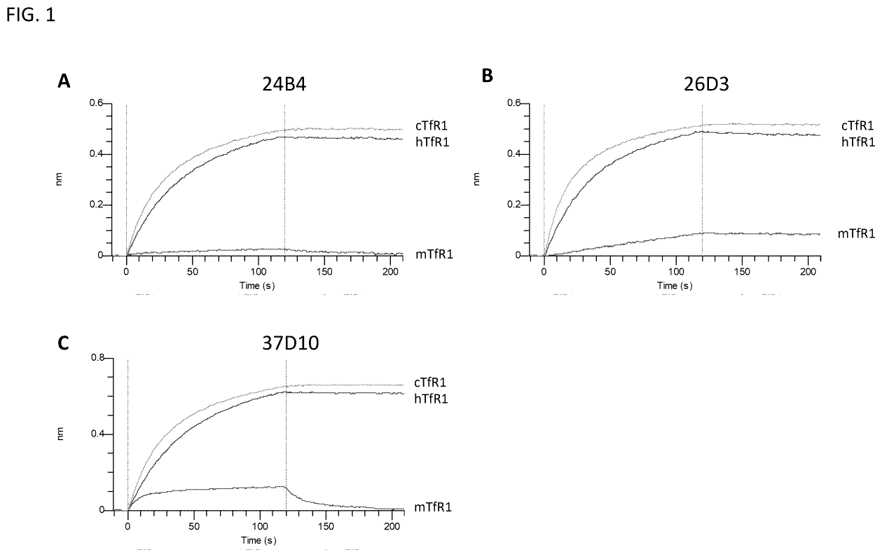

FIG. 1 shows the results of BLI measurements for three selected clones provided as examples, with binding measured directly in the crude hybridoma supernatant. Briefly, mouse IgG antibody clones in hybridoma supernatants, diluted 1:1 in running buffer (PBS, 0.02% TWEEN®-20 and 0.01% BSA), were captured on anti-mouse capture biosensors (anti-mouse capture, AMC, Molecular devices, Cat. 18-5580). Next, sensors with immobilized IgGs were briefly washed for 10 s before incubation in running buffer to establish a baseline signal. Association to target antigens were measured by incubating sensors for 120 s in wells of the assay plate containing the following concentrations of respective target antigen: 500 nM mTfR1, 250 nM hTfR1 and 250 nM cTfR1. All proteins were diluted in running buffer. Target dissociation was measured by incubating the biosensors in running buffer for 90 s. All tested clones, i.e. 24B4, 26D3 and 37D10, bind to both human and cynomolgus TfR1 but very weakly to mouse TfR1. Overall, most clones showed more cross-reactivity towards human and cynomolgus TfR1 than against mouse TfR1.

### Sequencing of Selected Clones

Clones of interest were cryopreserved and sequenced by whole transcriptome shotgun sequencing. Among the sequenced hybridoma clones were clones denoted 26D3, 24B4 and 37D10. The amino acid sequences obtained for the respective heavy chain variable (VH) and light chain variable (VL) regions of these antibodies are given in Table 2 below:

TABLE 2
Variable region amino acid sequences for selected primary antibodies

| | | SEQ |
|---|---|---|
| | Antibody | ID |
| Region | Amino acid sequence | NO: |
| | 26D3 | |
| VH | EVQLQQFGVELVKPGASVKISCKASGYIFTDYNMDWVKQSHGKSLEWIGDIN | 65 |
| | PDYDTTSYNEKFKGKATLTVDKSSSTAYMELRSLTSEDTAVYYCARGGYSGSSY | |
| | YHPMDYWGQGTSVTVSS | |
| VL | DIVMSQSPSSLAVSVGEKITMSCKSSQSLLYSTNQKNYLAWYQQKPGQSPELLI | 66 |
| | YWASTRESGVPDRFTGSGSGTDFTLTISNVRAEDLAVYYCQQYFIYPRTFGGGT | |
| | KLEIK | |
| | 24B4 | |
| VH | EVQLQQFGAELVKPGTSVKISCKASGYTFTDYNMDWVKQGHGKGLEWIGDIN | 67 |
| | PNYDTTSYSQKFKGKATLTVDKSSSTAYMELRSLTSEDTAVYYCARSEAGNYYWY | |
| | FDVWGAGTTVTVSS | |
| VL | DIVMSQSPSSLAVSVGEKVTMSCKSSQSLLYSSNRKNYLAWYROKPGQSPKLLI | 68 |
| | YWASTRESGVPDRFTGSGSGTDFTLTISSVKAEDLAVYSCQQYYNYPYTFGGGT | |
| | KLEIK | |
| | 37D10 | |
| VH | QVQLQQSGAELVRPGTSVKISCKASGYAFTNYWLGWVKQRPGHGLEWIGDIF | 69 |
| | PGSDNTYYNEKFKGKATLTADKSSSTAYMQLSSLASEDSAVYFCARSGNFYAMD | |
| | YWGQGTSVTVSS | |
| VL | ENVLTQSPAIMSASPGEKVTMTCSASSSVNYMNWYQQKSSTFPKLWIYDTSKL | 70 |
| | ASGVPGRFSGSGSGKFYSLTISSMEAEDVATYYCFQGSGYPFTFGSGTKLEIK | |

The complementarity determining regions (CDRs) of these antibodies were identified using the Kabat definition, and are given in Table 3 below.

TABLE 3
CDR sequences of primary antibodies

| Antibody | VHCDR1 | VHCDR2 | VHCDR3 |
|---|---|---|---|
| 26D3 | DYNMD | DINPDYDTTSYNEKFKG | GGYSGSSYYHPMDY |
| | (SEQ ID NO: 10) | (SEQ ID NO: 11) | (SEQ ID NO: 12) |
| 24B4 | DYNMD | DINPNYDTTSYSQKFKG | SEAGNYYWYFDV |
| | (SEQ ID NO: 10) | (SEQ ID NO: 34) | (SEQ ID NO: 35) |
| 37D10 | NYWLG | DIFPGSDNTYYNEKFKG | SGNFYAMDY |
| | (SEQ ID NO: 38) | (SEQ ID NO: 39) | (SEQ ID NO: 40) |
| | VLCDR1 | VLCDR2 | VLCDR3 |
| 26D3 | KSSQSLLYSTNQKNYLA | WASTRES | QQYFIYPRT |
| | (SEQ ID NO: 13) | (SEQ ID NO: 14) | (SEQ ID NO: 15) |
| 24B4 | KSSQSLLYSSNRKNYLA | WASTRES | QQYYNYPYT |
| | (SEQ ID NO: 36) | (SEQ ID NO: 14) | (SEQ ID NO: 37) |
| 37D10 | SASSSVNYMN | DTSKLAS | FQGSGYPFT |
| | (SEQ ID NO: 41) | (SEQ ID NO: 42) | (SEQ ID NO: 43) |

In accordance with the present disclosure, the VH and VL regions of the identified antibodies are mutated to introduce cysteine residues for the provision of a disulfide bridge between the VH and VL regions. The resulting sequences, variously denoted “disulfide stabilized variants”, “DS stabilized variants”, “DS versions” or similar herein, are given in Table 4.

TABLE 4
Disulfide stabilized variants of primary antibodies

| | | SEQ |
|---|---|---|
| | Antibody | ID |
| Region | Amino acid sequence | NO: |
| | 26D3_DS | |
| VH | EVQLQQFGVELVKPGASVKISCKASGYIFTDYNMDWVKQSHGKCLEWIGDIN | 102 |
| | PDYDTTSYNEKFKGKATLTVDKSSSTAYMELRSLTSEDTAVYYCARGGYSGSSY | |
| | YHPMDYWGQGTSVTVSS | |
| VL | DIVMSQSPSSLAVSVGEKITMSCKSSQSLLYSTNQKNYLAWYQQKPGQSPELLI | 112 |
| | YWASTRESGVPDRFTGSGSGTDFTLTISNVRAEDLAVYYCQQYFIYPRTFGCGT | |
| | KLEIK | |
| | 24B4_DS | |
| VH | EVQLQQFGAELVKPGTSVKISCKASGYTFTDYNMDWVKQGHGKCLEWIGDIN | 103 |
| | PNYDTTSYSQKFKGKATLTVDKSSSTAYMELRSLTSEDTAVYYCARSEAGNYYWY | |
| | FDVWGAGTTVTVSS | |
| VL | DIVMSQSPSSLAVSVGEKVTMSCKSSQSLLYSSNRKNYLAWYROKPGQSPKLLI | 113 |
| | YWASTRESGVPDRFTGSGSGTDFTLTISSVKAEDLAVYSCQQYYNYPYTFGCGT | |
| | KLEIK | |
| | 37D10_DS | |
| VH | QVQLQQSGAELVRPGTSVKISCKASGYAFTNYWLGWVKQRPGHCLEWIGDIF | 104 |
| | PGSDNTYYNEKFKGKATLTADKSSSTAYMQLSSLASEDSAVYFCARSGNFYAMD | |
| | YWGQGTSVTVSS | |
| VL | ENVLTQSPAIMSASPGEKVTMTCSASSSVNYMNWYQQKSSTFPKLWIYDTSKL | 114 |
| | ASGVPGRFSGSGSGKFYSLTISSMEAEDVATYYCFQGSGYPFTFGCGTKLEIK | |

### Example 2

### In Vitro Binding to Human and Cynomolgus TfR1 and Epitope Screen

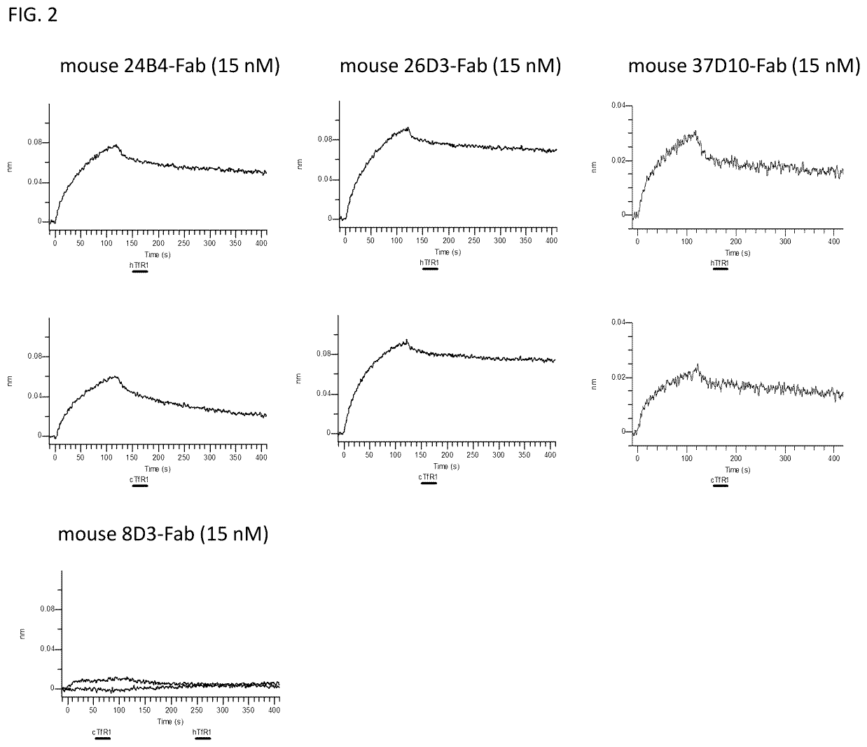

A more detailed binding analysis by BLI was performed on purified, selected antibodies. Binding of Fab fragments from the murine antibodies 26D3, 24B4 and 37D10 to human TfR1 and cynomolgus TfR1 was investigated. For example, the BLI instrument Octet Red384 was used to measure binding between immobilized TfR1 and the tested Fab fragments in solution. Antibody binding to TfR1 was measured with TfR1 complexed to the human transferrin ligand (Tf). Tf/TfR1-complexes were formed on streptavidin biosensors by first loading sensors with biotinylated human holo-transferrin followed by a complex-formation step by capturing either hTfR1 or cTfR1 on the sensors. Final complex density on the sensors was similar for both hTfR1 and cTfR1. Antibody binding to TfR1 was measured during an association phase of 120 s and a dissociation phase of 300 s. FIG. 2 shows sensorgrams for 15 nM of each of 24B4-Fab, 26D3-Fab and 37D10-Fab, as well as for a Fab derived from the known TfR1 binding antibody 8D3 (Boado et al (2009), Biotechnol Bioeng 102:1251-1258). The data indicate a similar binding profile against human and cTfR1 for both 24B4-Fab and 26D3-Fab, and cross-reactive binding to both species is also detected for 37D10-Fab, while no significant binding of 8D3-Fab against human or cynomolgus TfR1 was detected. Importantly, the experiment shows that 24B4-Fab, 26D3-Fab and 37D10-Fab all bind to TfR1 when the natural ligand transferrin is in complex with TfR1.

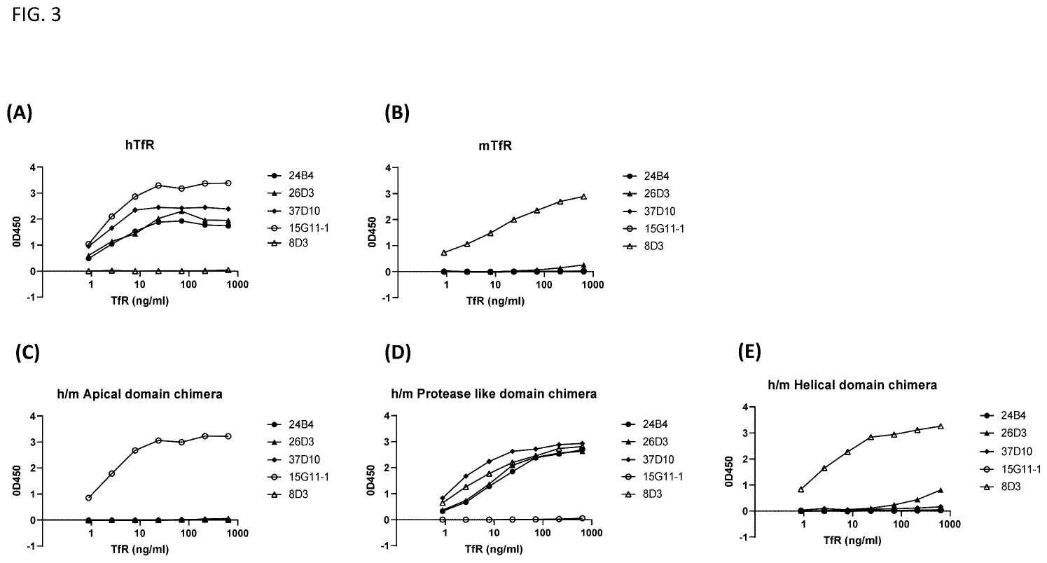

Next, an ELISA experiment showed that antibodies 26D3, 24B4 and 37D10 bind to the protease-like domain of TfR1. In the ELISA experiment, human, mouse or three different chimeric TfR1 receptors were used to coat ELISA plates (FIG. 3). The ELISA protocol was slightly modified as follows from the indirect ELISA described in Example 1. Briefly, ELISA plates were coated with the following His-tagged antigens at 1 μg/ml: ectodomain of human TfR1 (His₁₀-hTfR1; SEQ ID NO:74), ectodomain of mouse TfR1 (His₁₀-mTfR1; SEQ ID NO:75), chimeric TfR1 consisting of human apical domain grafted on mouse TfR1 ectodomain (h/m apical domain chimera, mhHD_TFR1; SEQ ID NO:76), chimeric TfR1 consisting of human helical domain grafted on mouse TfR1 ectodomain (h/m helical domain chimera, mhHD_TfR1; SEQ ID NO: 77) or chimeric TfR1 consisting of human protease like domain grafted on mouse TfR1 ectodomain (h/m protease-like domain chimera, mhPLD_TfR1; SEQ ID NO: 78). The coated plates were then blocked. Dilution series of mouse IgG of the analyzed antibodies were prepared in PBS and incubated on the ELISA plates. Unbound antibodies were then washed off before incubating wells with a HRP-conjugated secondary, anti-mouse-IgG for 1 h. Plates were then washed again before addition of HRP substrate TMB for development and detection of antibody binding to the wells. TMB development was stopped by adding 0.5 M H₂SO₄ to the wells and ELISA responses measured as the OD at 450 nm in an ELISA plate reader. As illustrated in FIG. 3, 26D3, 24B4 and 37D10 only bind hTfR1 (A) and not mTfR1 (B). There is no binding of 26D3, 24B4 or 37D10 to the construct with the human apical domain grafted onto the rest of the mTfR1 ectodomain (C). The control antibody 15G11-1 (Yu et al (2014), Sci Transl Med 6:261ra154) known to bind to the human apical domain shows binding to the h/m apical domain chimera as expected (C). In addition, 26D3, 24B4 and 37D10 bind to the h/m protease-like domain chimera (D), but not to any of the plates coated with the other chimeric receptors (C and E). Further, the control antibody 8D3, with an epitope in the apical domain of mTfR1, binds to all plates coated with TfR1 antigens including this domain, namely mTfR1 (B), h/m protease-like domain chimera (D) and h/m helical domain chimera (E), In summary, the experiment demonstrates that the epitope or epitopes for 26D3, 24B4 and 37D10 lie(s) predominantly within the protease-like domain of hTfR1, and that this is in contrast to the control antibodies 15G11-1 and 8D3.

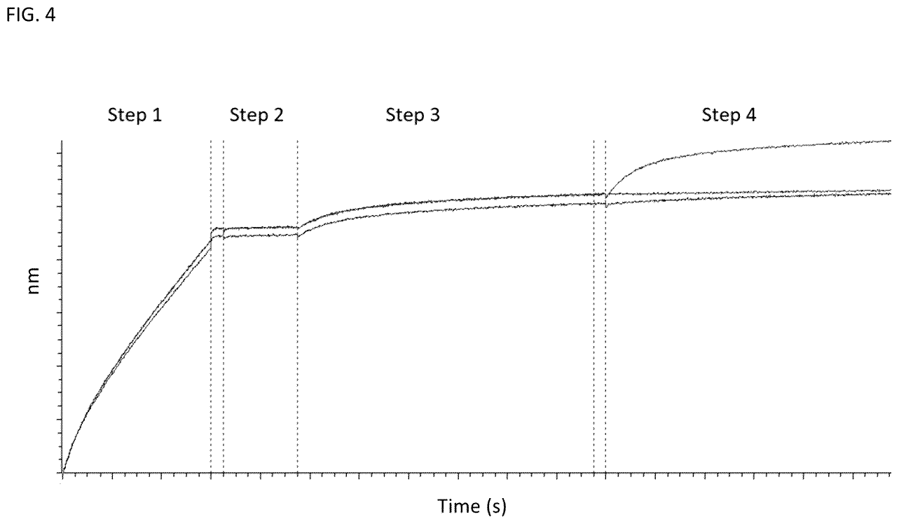

In a further BLI experiment carried out for the purpose of epitope binning (binding competition), it was then shown that binding by both 26D3 and 24B4 is targeted to the same or overlapping regions of hTfR1, with an epitope located outside the apical domain (FIG. 4). The epitope binning experiment by BLI was conducted on an Octet Red384 instrument (ForteBio) by first (Step 1) immobilizing biotinylated hTfR1 to streptavidin biosensors (High precision biosensors, ForteBio). Next (Step 2), a washing step was carried out. Then (Step 3), hTfR1 loaded sensors were incubated in either buffer (non-competitive reference) or 200 nM of the respective antibody (Ab) to form hTfR1: Ab complexes on the sensors. Finally (Step 4), sensors with free hTfR1 (reference) or respective preformed hTfR1: Ab complex was incubated in 200 nM of respective antibody to measure binding to hTfR1 in complex with the competing antibody. FIG. 4 shows representative BLI sensorgrams obtained during the indicated main assay steps. The signal in Step 4 is indicative of the degree of competition between the two analyzed antibodies. If the antibodies compete for binding to the same or overlapping epitope, there is no increase in the signal of the sensorgram in Step 4. Conversely, if the two tested antibodies bind to distinct and different epitopes, there will be an increased signal from Step 4.

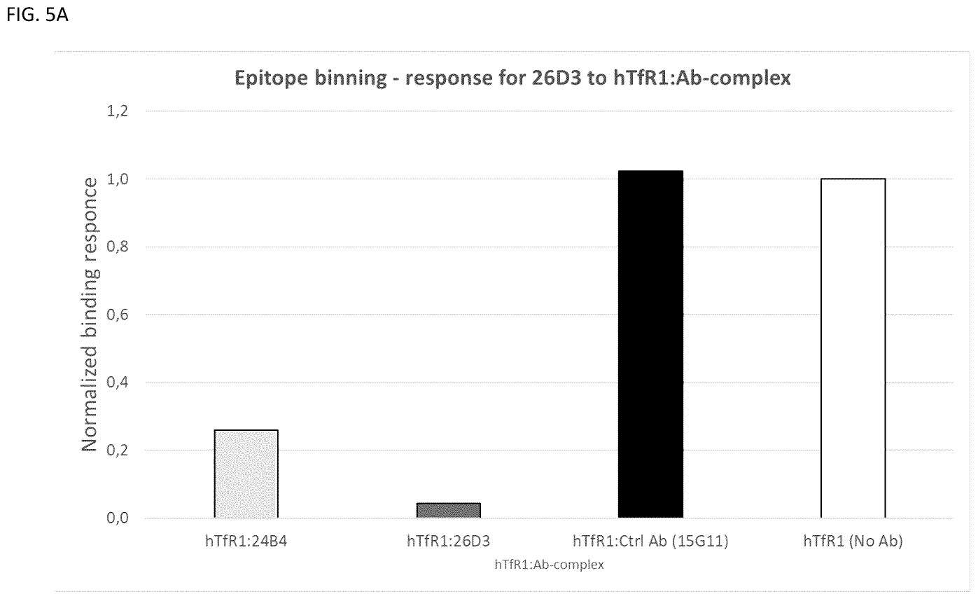

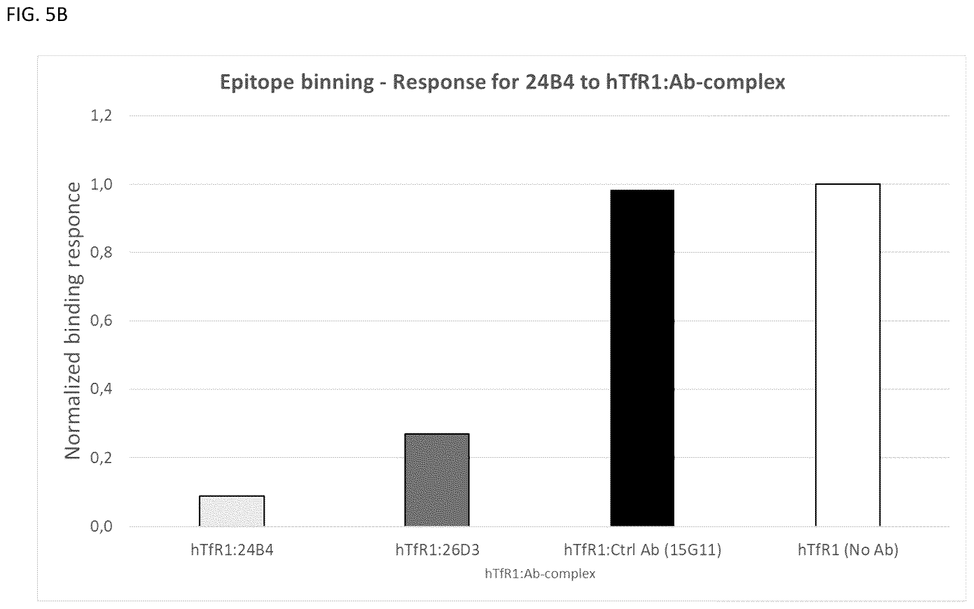

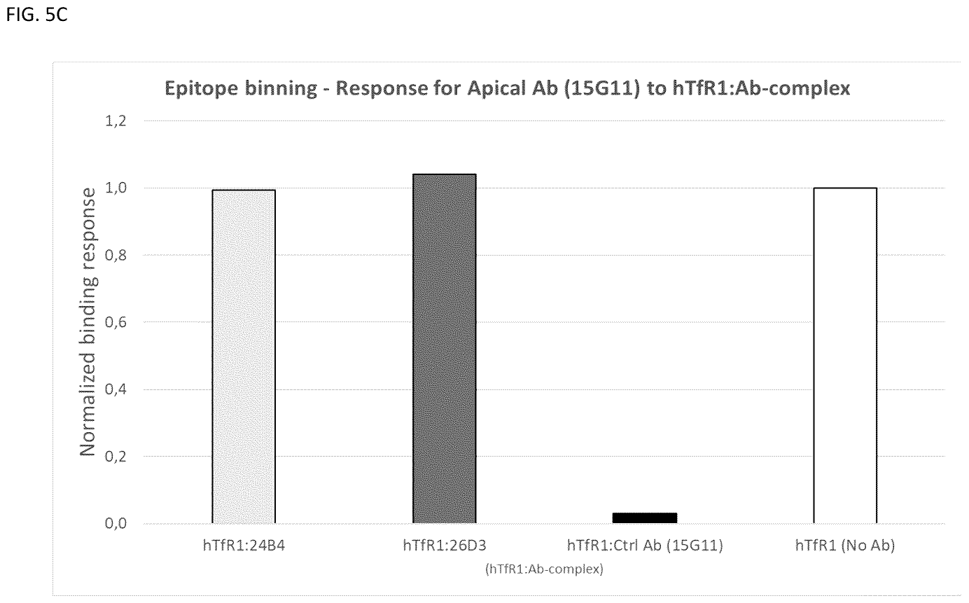

The results of competitive screening of antibody binding to epitopes on hTfR1 by epitope binning as described above is illustrated in FIG. 5. Antibodies 26D3 (dark grey bars) and 24B4 (light grey bars) were shown to bind to an overlapping epitope, which is distinct from the hTfR1 apical domain epitope of control antibody 15G11-1 (black bars). FIG. 5A shows that the binding response for 26D3 is reduced by over 70% when hTfR1 is in complex with 24B4. As expected, binding of 26D3 to pre-formed hTfR1:26D3-complex is nearly fully inhibited, illustrating that it blocks itself. Similarly, FIG. 5B shows that the binding response for 24B4 is 70% lower when hTfR1 is in complex with 26D3 and nearly fully inhibited by itself. Both 24B4 and 26D3 retain the full binding response to hTfR1 when hTfR1 is in complex with the control antibody 15G11-1, which has its binding epitope within the apical domain of hTfR1 (FIGS. 5A and 5B, black bars). As shown in FIG. 5C, the control antibody 15G11-1 has similar binding responses to the apical domain of hTfR1, regardless of whether it is tested against hTfR1 without competition antibody or when the receptor is in complex with 24B4 or 26D3. In FIG. 5, all responses were normalized to the respective antibody's maximal binding response to free hTfR1.

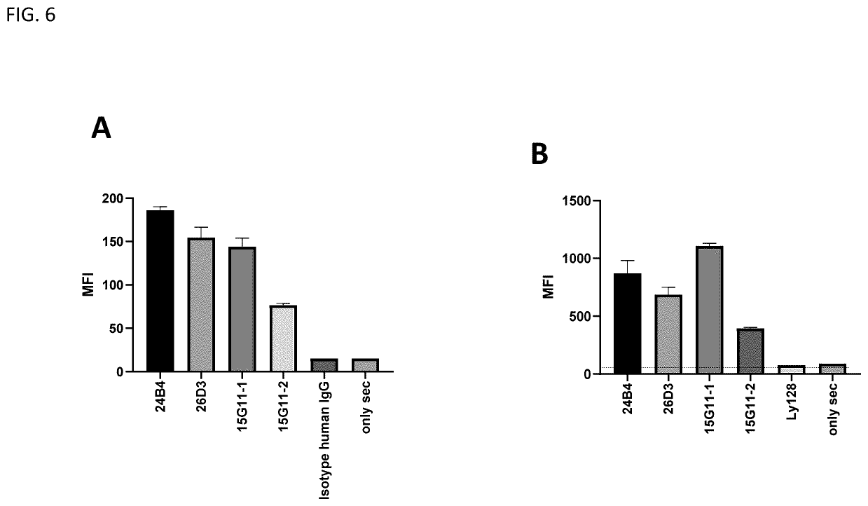

Furthermore, antibody binding to endogenous hTfR1 on brain endothelial cells was studied. Binding to endogenous hTfR1 on cell surfaces was monitored using flow cytometry and human hCMEC/D3 cells (Weksler et al (2013), Fluids Barriers CNS 10:16), which are known to express significant levels of hTfR1 on their surface. Cells that stained positively were plotted and the mean fluorescence intensity (MFI) is shown in FIG. 6. Both FIG. 6A (IgG1 antibodies) and 6B (Fab fragments) show that cells were positively stained for hTfR1 with 24B4 and 26D3 to a similar degree (MFI) compared to the positive control antibody 15G11-1 having a high hTfR1 affinity and to a higher degree than the low affinity control antibody 15G11-2 (Yu et al (2014), supra). No background staining was detected with the negative isotype control (FIG. 6A) or the non-related Fab fragment Ly128 (FIG. 6B). These data illustrate that both 24B4 and 26D3 bind to hTfR1 expressed on a cell surface.

### Example 3

### Competition for hTfR1 Binding with Ferritin and Transferrin

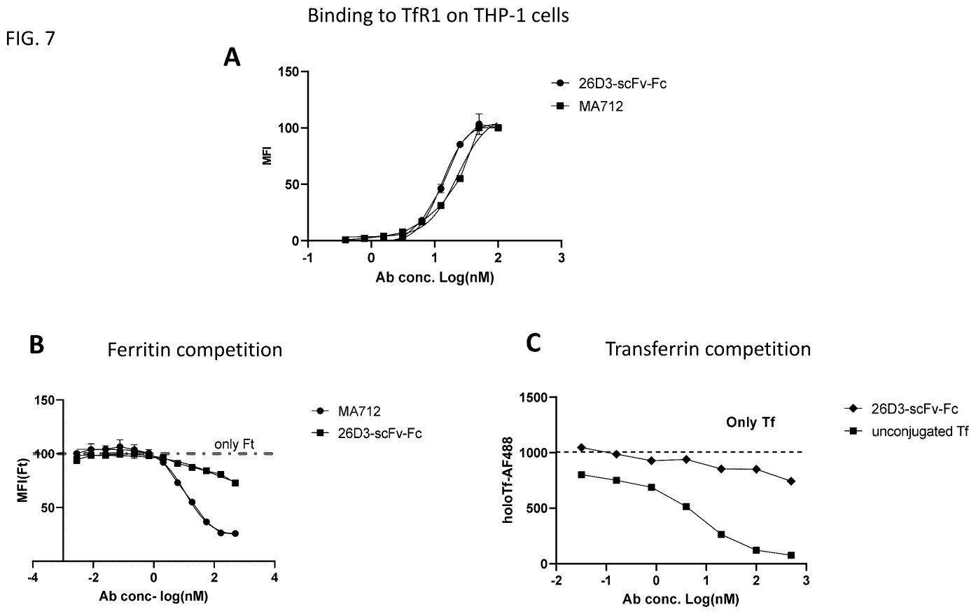

The unique binding to hTfR1 of the binders according to the disclosure, binding to the protease-like domain of hTfR1 and identified as described in Example 1, was evaluated for competition with natural TfR1 ligands ferritin (Ft) and transferrin (Tf). In order to test ferritin competition with antibody, the human monocytic cell line THP-1 (Sigma/ECACC) was used. Binding of the scFv-Fc format (see Example 4 below) and control antibody (M-A712) to hTfR1 on the THP-1 cell surface was confirmed, as shown in FIG. 7A. For evaluating the competition between ferritin and the disclosed binders, cells were incubated with serially diluted test binders along with ferritin from human liver (BioRad, 4420-4804) for 1 h at 4° C. After incubation, ferritin that had bound to hTfR1 on the cell surface was captured using a primary sheep antibody against human liver ferritin (BioRad, AHP2179G) and analyzed using flow cytometry. The results are displayed in FIG. 7B, and show that the 26D3 scFv-Fc does not compete with ferritin on the cell surface, whereas the control antibody anti-CD71, clone M-A712, known to bind to the same epitope on hTfR1 as Ft (Maier et al (2016), Mol Ther Nucleic Acids 5: e321) clearly competes with Ft binding. Also for the identified 26D3 hTfR1 binder, the impact on Ft binding is much less, illustrating that 26D3 has a different epitope on hTfR1 than the binding site for Ft (FIG. 7B).

For transferrin competition, K562 lymphoblast cells (Sigma/ECACC) were used. Cells were incubated with serially diluted test constructs along with Alexa Fluor 488 conjugated, human holo-transferrin (Thermo Fisher; T13342) and incubated for 1 h at 4° C. Transferrin bound to hTfR1 on cell surfaces was captured using flow cytometry, and the mean fluorescence intensity was plotted. FIG. 7C shows that there is no competition between the 26D3 binder and transferrin. When non-labeled (unconjugated) Tf was used as positive control for competition, the binding of labeled (AF488) Tf signal was reduced in a concentration dependent way. The experiment illustrates that a binder directed against the protease-like domain of TfR1 does not compete directly for the same epitope as transferrin.

Overall, this example shows that binding of 26D3 to hTfR1 does not negatively affect the ability of the two endogenous ligands ferritin and transferrin to bind to the receptor.

### Example 4

### Humanization of hTfR1 Binder 26D3

The Fab sequence of mouse antibody 26D3, identified and characterized as described in Examples 1-3, was analyzed and an in silico model of the 26D3 Fab 3D structure was generated using Bioluminate Software (Schrödinger). This murine Fab model was used as input for humanization. In this process, the CDRs of the VH and VL regions of 26D3 (see Table 3; SEQ ID NO:10-15) were grafted in silico into various human variable domains and some residues were back mutated to murine framework at some positions. Three variants having the fewest back mutations and otherwise desirable characteristics were generated and extracted from the software. One such humanized variant was selected for expression and denoted h26D3. h26D3 has the VH region sequence defined in SEQ ID NO:44 and the VL region sequence defined in SEQ ID NO:58. According to this disclosure, a DS version of h26D3 has the VH and VL amino acid sequences SEQ ID NO:88 and 105, respectively.

The humanized version h26D3 and the murine original sequence 26D3 were both expressed as His-tagged Fabs by transient transfection of Chinese Hamster Ovary cells (ExpiCHO; Thermo Fisher Scientific) according to the manufacturer's instructions. The harvested supernatant was purified using HiTrap IMAC Sepharose FF (Cytiva) followed by a size exclusion chromatography on HiLoad Superdex 200 μg 26/600 (Cytiva). The following buffers were used: Ni-NTA wash buffer: 20 mM Tris pH 8.0, 10 mM imidazole and 200 mM NaCl; Ni-NTA elution buffer: 20 mM Tris pH 8.0, 200 mM NaCl and 500 mM imidazole; size-exclusion buffer (SEC): 1×dPBS (Thermo Fisher).

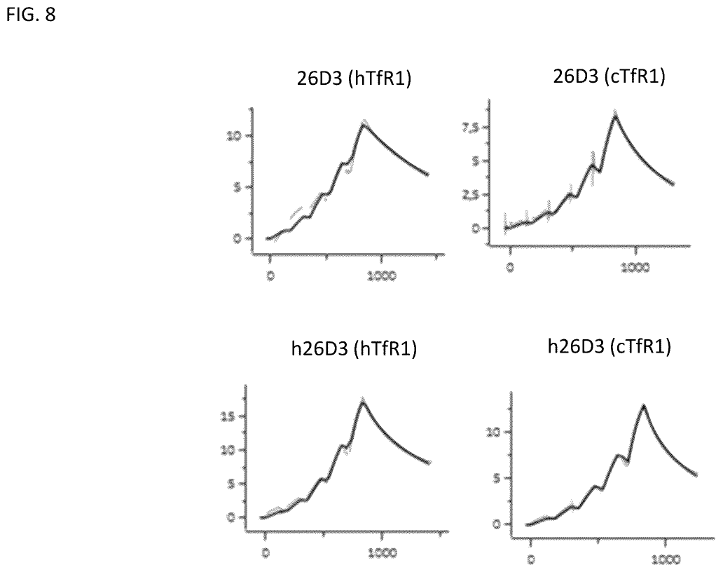

Binding of the purified Fabs to human and cynomolgus TfR1 was evaluated using surface plasmon resonance (SPR) on a Biacore 8K instrument (Cytiva) and the results are shown in FIG. 8. 1 μg/ml of human TfR1 (truncated hTfR1 of SEQ ID NO: 86) or cynomolgus TfR1 (truncated cTfR1 of SEQ ID NO:87) was immobilized on a Cm5 sensor chip (Cytiva, #BR100399) using the amine coupling kit type 2 (Cytiva, #BR100633) according to the manufacturer's instruction. The h26D3 and 26D3 Fabs were injected over the chip using a 2-fold dilution series in five steps starting at 25 nM. The interaction was measured using the single cycle kinetics method with a contact time of 120 s at a flow rate of 30 μl/ml followed by a dissociation time of 600 s. Regeneration of the surface between cycles was done by injecting 3M MgCl₂. The binding data were fitted to a 1:1 interaction model. The Fabs were diluted in HBS-EP+ (Cytiva, #BR100669). Experiments were performed at 25° C. The data confirm that the humanized variant of 26D3, i.e. h26D3, retained binding capacity for human and cynomolgus TfR1 (FIG. 8). The kinetic parameters obtained in the experiment are given in Table 5 below.

TABLE 5
SPR analysis of murine and humanized
26D3 Fabs vs. hTfR1 and cTfR1

| Tested binder | Target | kₐ (1/Ms) | k_{d} (1/s) | K_{D} (M) |
|---|---|---|---|---|
| 26D3 | hTfr1 | 2.8 × 10⁶ | 1.0 × 10⁻³ | 3.6 × 10⁻⁹ |
| 26D3 | cTfR1 | 5.7 × 10⁶ | 6.2 × 10⁻³ | 1.0 × 10⁻⁸ |
| h26D3 | hTfr1 | 1.2 × 10⁶ | 4.5 × 10⁻³ | 3.2 × 10⁻⁹ |
| h26D3 | cTfR1 | 2.7 × 10⁶ | 1.5 × 10⁻³ | 5.8 × 10⁻⁹ |

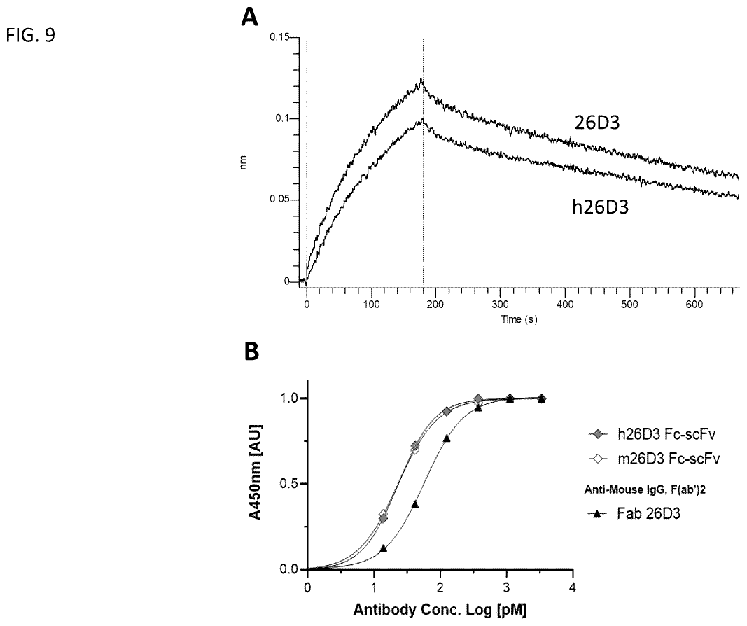

Both murine 26D3 and the humanized variant h26D3 were converted to the scFv format and confirmed to have maintained target binding as scFv (FIG. 9). Murine and humanized 26D3 were reformatted to scFv (SEQ ID NO:79 and SEQ ID NO: 80 respectively) and produced as monovalent Fc-fused scFv antibody fragments by employing the knob-into-hole (KiH) technology. In this format, one scFv fragment is fused only to the knob half of the Fc (SEQ ID NO:81), while the hole half of Fc (SEQ ID NO: 82) is left unfused. The resulting antibody format is a one-armed scFv-Fc. The 26D3 scFv fused to the knob half of the Fc has the complete amino acid sequence SEQ ID NO:83, whereas the h26D3 scFv fused to the knob half of the Fc has the complete amino acid sequence SEQ ID NO:84. The binding profiles for murine and humanized 26D3 in this scFv format are similar and confirm binding activity in the scFv format. Binding responses agree with those of the antibody in Fab format. This was confirmed by several methods, including a kinetic experiment using BLI (results shown in FIG. 9A) and an ELISA (results shown in FIG. 9B). Binding kinetics for murine and humanized 26D3-scFv-Fc were measured by BLI by first immobilizing biotinylated hTfR1 to streptavidin biosensors (Fortebio). Sensors were then washed in buffer (Kinetics buffer, Fortebio) before measuring association of 26D3-scFv-Fc (murine) and h26D3-scFv-Fc (humanized) at 25 nM concentrations followed by a 500 s dissociation phase. In the ELISA experiment, hTfR1 was used to coat the plates for standard binding ELISA experiments using the protocol for indirect ELISA described in Example 1.

### Example 5

### Crystallization and Structure Determination of h26D3-Fab in Complex with hTfR1

This example describes crystallization of a complex between h26D3-Fab and hTfR1 and determination of the binding interface. Ectodomain of human TfR1 (SEQ ID NO: 74) was expressed by transient transfection of human embryonic kidney cells (Expi297; Thermo Fisher Scientific) according to the manufacturer's instructions. The harvested supernatant was purified using HiTrap IMAC Sepharose FF (Cytiva) followed by size exclusion chromatography on HiLoad Superdex 200 μg 26/600 (Cytiva). The buffers used and purification of the humanized Fab were as described in Example 4.

The formation of a complex between humanized h26D3-Fab and hTfR1 was done by mixing of the two components at a molar ratio of 1:1 in 1x dPBS and incubation at room temperature for 1 h. Subsequently, the complex was purified using size exclusion chromatography on HiLoad Superdex 200 μg 26/600 (Cytiva) as described in Example 4.

Crystallization was performed using a stock solution of hTfR1-h26D3 at 15 mg/ml in PBS which was diluted to 4 mg/ml in PBS supplemented with 4 mM B-mercaptoethanol. A 100+100 nl drop was set up using the additive screen in reservoir: 0.1 M sodium potassium phosphate pH 6.5, 10% PEG 3000, 0.05% dichloromethane and 2 mM B-mercaptoethanol. The crystal was flash-frozen in reservoir solution supplemented by 8% glycerol and 16% PEG 400.

X-ray data collection and refinement were performed as follows. Data was collected to 3.87 Å at Diamond Light Source beamline 104. The beamline was equipped with a DECTRIS Eiger2 XE 16M detector. The data set was integrated using XDS (Kabsch (2010), Acta Crystallogr D Biol Crystallogr 66:125-132) with STARANISO anisotropic scaling (Tickle et al (2018), Global Phasing Ltd) and diffracted to 3.87 Å along the c* direction of the reciprocal lattice, and to 4.82 Å in the a/b* plane. Three complexes were found in the asymmetric unit. The structure was refined using the Buster refinement software and model building was carried out in Coot. Data collection and refinement parameters and statistics are given in Table 6 below.

TABLE 6
X-ray diffraction data collection and refinement statistics

| Resolution (Å) | 110.81-3.87 | (4.27-3.87) |
|---|---|---|
| Wavelength (Å) | 0.97950 | |
| Space group | P3121 | |
| Unit cell (Å) | a = b = 127.95, c = 472.91 | |
| Spherical completeness (%) | 63.9 | (12.7) |
| Ellipsoidal completeness (%) | 93.8 | (74.0) |
| No. of observations/unique reflections | 546922/27520 | (27874/1378) |
| Redundancy | 19.9 | (20.2) |
| <I/σ(I)> | 9.8 | (1.7) |
| CC(1/2) (%) | 99.9 | (81.5) |
| Rmerge (I) (%) | 20.6 | (200.9) |
| Rpim (I) (%) | 6.6 | (62.8) |
| Rmodel (F) (%) | 26.0 | (31.6) |
| Rfree (F) (%) | 31.4 | (34.3) |
| No. of non-hydrogen atoms | 24858 | |
| No. of water molecules | 0 | |
| rms deviations from ideal geometry | | |
| Bond lengths (Å) | 0.009 | |
| Bond angles (°) | 1.1 | |
| Mean B-factor protein chain | 76.9/110.0/103.0/132.2/ | |
| A/B/C/D/E/F/G/H/L (Å²) | 134.4/87.2/101.0/79.9/83.4 | |
| Mean B-factor glycosylations (Å²) | 70.7 | |
| Ramachandran plot quality | | |

| | |
|---|---|
| Favored regions (%) | 93.4 |
| Allowed regions (%) | 4.3 |
| Outliers (%) | 2.3 |

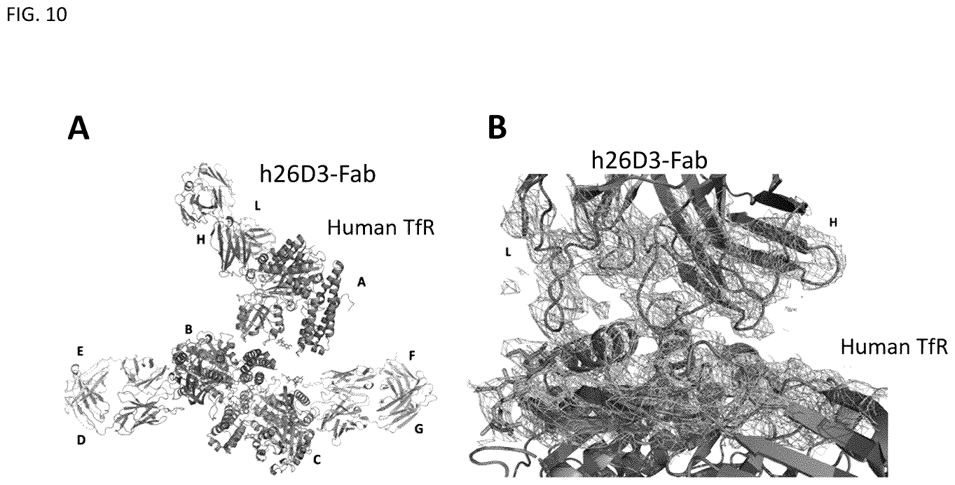

The final, refined structure of the complexes showing the overall folds is depicted in FIG. 10. As shown in FIG. 10A, there were three independent complexes in the asymmetric unit. The chain names as used in the coordinate files are indicated. FIG. 10B shows an example of the electron density contoured at the interface between hTfR1 and heavy/light chain of h26D3-Fab. The protein chains are drawn in cartoon representation while sugar moieties are shown in stick representation. The binding interface interaction between h26D3 and human TfR1 was extracted from the X-ray structure and described in the following to provide information about the precise binding of h26D3 to human TfR1.

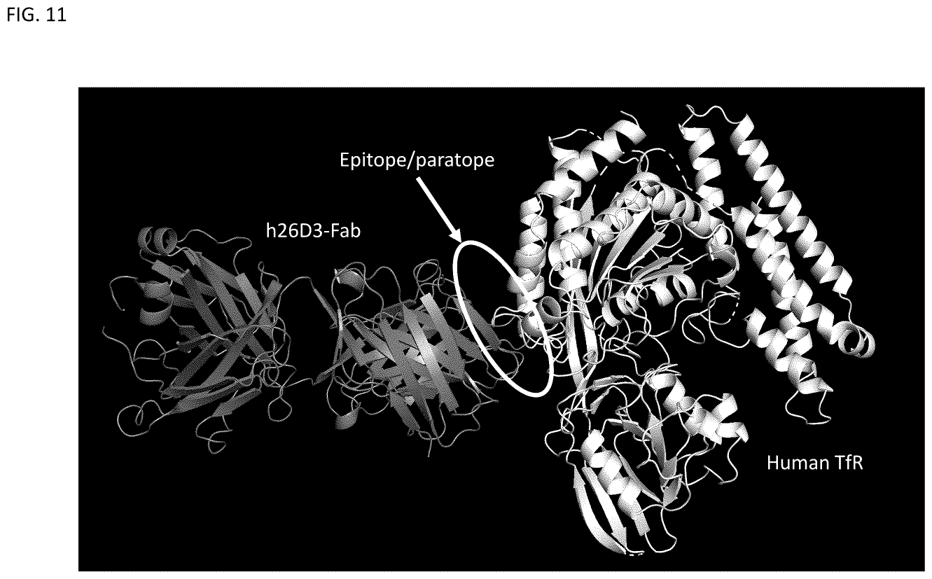

The binding interface between hTfR1 and h26D3-Fab is depicted in FIGS. 10 and 11, and interaction was observed between the amino acid residues indicated in Table 7.

TABLE 7
Amino acid residues involved in interaction
between h26D3 and hTfR1

| | | Human TfR1 |
|---|---|---|
| h26D3 heavy chain | | |
| 50ASP | interacts with | 150Asn |
| 57Thr | interacts with | 150Asn |
| 59Ser | interacts with | 150Asn |
| 105Ser | interacts with | 154Pro |
| 105Ser | interacts with | 159Ser |
| 105Ser | interacts with | 161Lys |
| 106Tyr | interacts with | 161Lys |
| 106Tyr | interacts with | 158Gly |
| 107Tyr | interacts with | 151Ser |
| h26D3 light chain | | |
| 32Ser | interacts with | 163Glu |
| 32Ser | interacts with | 385Lys |
| 33Thr | interacts with | 160GIn |

Table 7 describes the key residues from both sides involved in the epitope/paratope interface as determined from the crystal structure. Additional residues in the vicinity are also likely to be important for the binding between h26D3 and hTfR1. In addition, as described in Example 9 below, several positions outside the observed binding interaction show important participation in binding of h26D3 to hTfR1.

In Table 8 below, the amino acids of human TfR1 that are involved in the respective interactions with h26D3, Ft and Tf are listed. Notably, no amino acids involved in the binding of h26D3 form part of any of the binding interfaces for the endogenous ligands. This illustrates that the binders of the present disclosure, as exemplified by h26D3, bind to hTfR1 outside the binding sites used by Ft and Tf.

TABLE 8
Amino acid residues in hTfR1 which
interact with the respective ligand

| h26D3 | Ferritin* | Transferrin# |
|---|---|---|
| 150Asn | 195Ser | 121Arg |
| 151Ser | 197Gln | 123Tyr |
| 154Pro | 199Ser | 125Asp |
| 158Gly | 201Ile | 126Asp |
| 159Ser | 208Arg | 622Val |
| 161Lys | 209Leu | 623Arg |
| 163Glu | 210Val | 626Asn |
| 385Lys | 212Leu | 629Arg |
| | 215Asn | 640Gln |
| | 343Glu | 643Tyr |
| | 343Lys | 651Arg |
| | 344Lys | 661Gly |
| | 348Asn | 662Asn |
| | 374Lys | 663Ala |
| | | 664Glu |
| | | 667Asp |
| | | 757Asp |
| | | 758Asn |

*Montemiglio et al (2019), Nat Commun 10:1121
#Eckenroth et al (2011), Proc Natl Acad Sci USA 108:13089

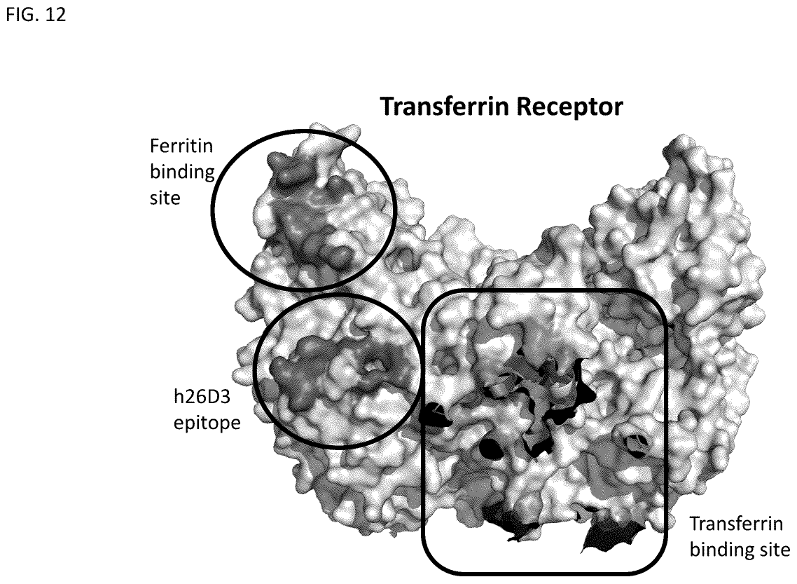

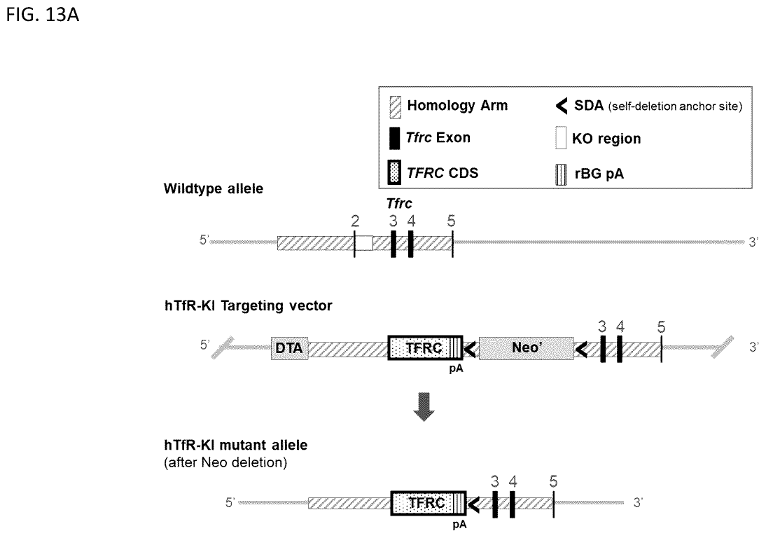

The different epitopes on the hTfR1 structure (pdb: 1SUV) are illustrated further in FIG. 12. As shown in FIG. 12, the Ft binding site is located on the apical domain of hTfR1, the Tf binding site is mainly located on the helical domain of hTfR1 and the h26D3 epitope is located on the protease-like domain of hTfR1. The structure illustrates that the different ligands and binder use distinct, specific surface areas on the hTfR1 structure. hTfR1 is a homodimer having two identical chains, and the epitopes are only indicated on one of these chains.

### Example 6

### Generation and Characterization of hTFR1 Knock-In Mice

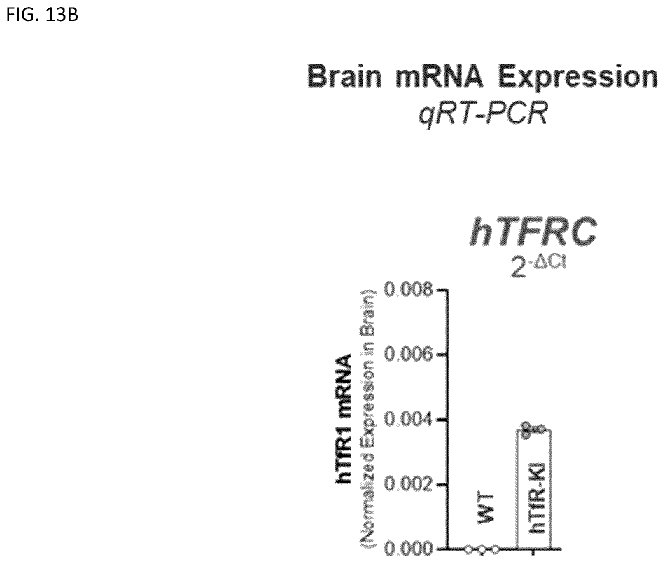

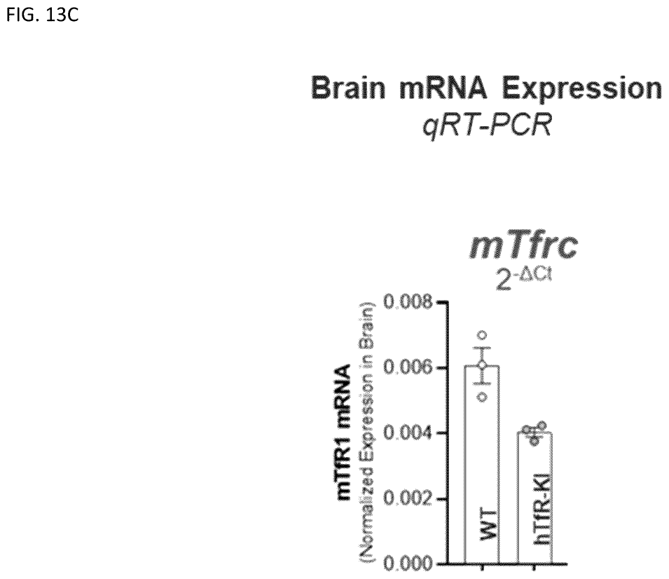

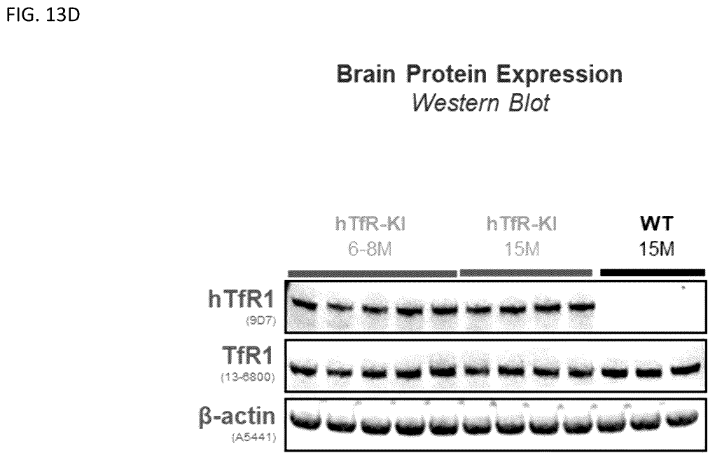

Human TfR1 knock-in (hTfR1-KI; TFR1C-KI) mice were generated by homologous recombination (experimental work performed at Cyagen US). A cDNA vector carrying the TFR1C (NCBI Reference Sequence: NM_001128148.3) ectodomain and murine Tfrc transmembrane and intracellular domain were introduced by pronuclear microinjection in C57BL/6N ES cells Tfrc. The coding region of Tfrc exon 2 plus partial intron 2 were replaced with the TFR1C chimeric cassette (FIG. 13A). Correct insertion of hTfR1 cDNA was verified by Southern blot and PCR. Transgene expression in hTfR1-KI mice was confirmed in brain tissue by qRT-PCR (FIG. 13B) and western blot (FIG. 13C), indicating endogenous expression levels. hTfR1-KI mice were maintained on a C57BL/6N background and only heterozygous hTfR1-KI mice were used for experiments.

### Example 7

### Brain Uptake of hTfR1 Binding Constructs In Vivo

To evaluate hTfR1-mediated brain uptake in vivo, monovalent Fc-scFv constructs (see Example 4) were produced for four different binding proteins. A known binder to hTfR1, 15G11-1, was used as a control (Yu et al (2014), supra). This hTfR1 binder has been described to be active in vivo and is used as a positive reference control for brain uptake. In addition, a construct containing a non-hTfR1 scFv binder based on the anti-amyloid B antibody mAb158 was designed and included as a negative control in the form of an Fc fusion construct (Fc-scFv158, also referred to as simply “158” here and in the FIG.s). The different Fc-scFv constructs were injected intravenously (i.v.) into hTfR1 knock-in (hTfR1-KI) mice produced as described in Example 6 (n=4 per construct) at equimolar doses of 30 nmol/kg (corresponding to approximately 2.3 mg/kg). Plasma and brain exposure was assessed 24 h after dose.

The animals were anaesthetized using isoflurane and terminal blood samples were collected from the orbital plexus into BD Microtainer K2EDTA tubes. The samples were inverted and centrifuged at 2400×g for 10 min at 4° C. Plasma was extracted and transferred to Eppendorf tubes and frozen at −80° C. Immediately following blood sampling, the abdomen of the animals was cut open and a cannula (21 G) was inserted into the left ventricle of the heart. A small cut was made in the right atrium and transcardial perfusion was performed with a minimum of 50 ml of cold PBS. Following perfusion, brains were extracted and the olfactory bulbs removed. The brains were separated into left and right hemispheres and cerebellum was removed from the left hemisphere, after which the left hemisphere was weighed and snap frozen on dry ice and stored at −80° C. until further preparation and analysis of the concentrations of injected constructs using a Meso Scale Discovery (MSD) based assay. The right hemispheres were placed in 4% formaldehyde and stored at 4° C. for 24 h, after which they were rinsed in cold PBS, transferred to cold 30% sucrose solution prepared in PBS and stored at 4° C. for further immunohistochemistry (IHC) processing (see Example 8 below).

For brain concentration measurements, frozen left hemispheres were thawed on ice and homogenized in TBS by automated bead homogenization. Triton was added to the homogenate to a final Triton concentration of 0.5% before centrifugation at 16 000×g, after which supernatants were collected. Brain and plasma concentrations of anti-hTfR1 Fc-scFv were determined using a custom build MSD assay detecting the human Fc. A standard 96-well MSD plate (MSD, #L15XA-3) was coated with 0.5 μg/ml goat anti-human IgG, Fcγ fragment specific antibody (Jackson Immuno Research Europe Ltd, #109-005-098) diluted in 1xPBS (Medicago AB, #09-9400-100). After incubation at 4° C. overnight, the plate was washed 4x in 1xPBS-TWEEN (Fisher Scientific, #09-9410-100) and blocked with 150 μl 1% BlockerA in PBS-TWEEN (MSD, #R93BA-4) per well. Samples and corresponding standards, ranging from 400 μM to 0.1 μM in 1:4 dilution steps, were added and incubated for 2 h and 900 rpm at room temperature. A 1 h incubation step with mouse anti-human IgG (Mabtech, 3850 Jan. 1000, MT145) diluted to 0.5 g/ml was included, followed by 1 h incubation of SULFO-TAG conjugated anti-mouse antibody (MSD, R32AC-1) diluted to 0.5 μg/ml when the plate was incubated for another hour at room temperature and 900 rpm. 150 μl MSD read buffer (MSD, #R92TC) per well was added before reading the plates in an MSD SECTOR Imager. Between each incubation step, a 4x wash in 1xPBS-TWEEN was performed. All antibodies and samples, except the coating antibody, were diluted in 1% Blocker A in PBS-TWEEN and added in a volume of 50 μl/well. The concentration of the analytes in the samples were evaluated with the MSD workbench software, using a 4PL curve fitting algorithm and curve weighting 1/Y2 for the standard curve. Statistical analysis was performed in GraphPad Prism (v. 9.0.0) using one way ANOVA with Tukey's post hoc test.

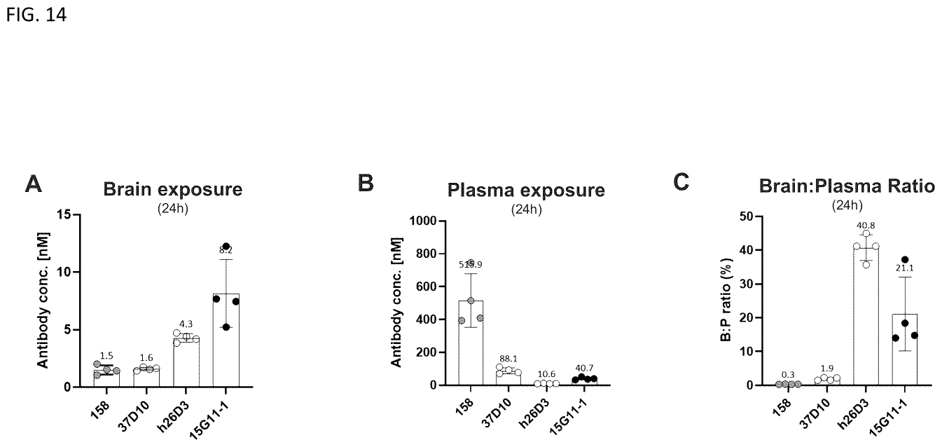

The results are shown in FIG. 14. As shown in FIG. 14A, substantially higher brain concentrations were observed for the two test constructs and the positive control 15G11-1, compared to the negative control (158) at 24 h after dose. As shown in FIG. 14B, the plasma concentrations of the two test constructs and the positive control 15G11-1 were lower at 24 h compared to that of 158, indicating that hTfR1 engagement leads to a faster plasma clearance. The brain-to-plasma concentration ratios are shown in FIG. 14C. The two test constructs and the positive control 15G11-1 showed a significantly enhanced brain exposure relative to plasma in comparison to the negative control. Taken together, the data supports hTfR1-mediated BBB transport in this experiment for the tested, novel hTfR1 binders.

### Example 8

### Immunohistochemistry Data on Brain Exposure

In vivo engagement of hTfR1 by the Fc-scFv construct was studied further using a qualitative immunohistochemistry (IHC) analysis. In brief, coronal brain sections at a thickness of 20 μm were obtained from PBS-perfused brain hemispheres of the mice described in Example 7 using a cryostat (Microm NX50 CryoStar, Epredia). The sections were collected on Superfrost plus slides (Menzel-Gläser, #J1800AMNZ) and air-dried prior to IHC. The brain sections were washed with PBS (pH 7.4) for 15 min and incubated in blocking buffer (5% BSA, 0.25% Triton-X in PBS) for 2 h at room temperature. To visualize i.v. dosed constructs, brain sections were incubated with a secondary goat anti-human IgG (heavy and light chain specific) conjugated to Alexa Fluor 488 (Invitrogen, #A11013) for 120 min at room temperature followed by 3×15 min wash in PBS. Slides were mounted with Fluoromount-G (Invitrogen, #00-4958-02) for imaging analysis. Confocal images from cerebral cortex were captured using a Leica Stellaris 5 confocal system equipped with a HC PL APO 40×/1.25 GLYC motCORR CS2 objective (Leica, #11506423).

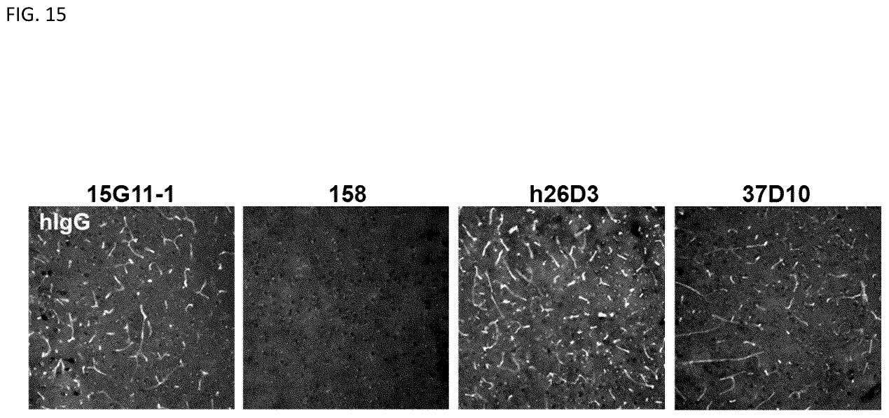

Distinct IHC immunofluorescence signals were observed in brain capillaries with positive reference module 15G11-1, while a minimal IHC signal was detected in brain sections from mice injected with negative control 158 (FIG. 15). Brain capillary IHC signal was observed for the two test constructs h26D3 and 37D10, of which h26D3 showed the strongest immunofluorescence signal, comparable to the positive control 15G11-1. Taken together, the MSD (Example 7) and IHC (this Example) analyses demonstrate that the hTfR1 binders of the disclosure in a scFv format exhibit an increased brain exposure in hTfR1-KI mice.

### Example 9

### Generation of Affinity Variants and Affinity Determinations

Several variants of the parental antibody h26D3 were generated by substituting tyrosine, tryptophan and aspartic acid residues in the CDRs one by one for alanine residues. The resulting variant VH regions were denoted HCl-HC13 and their amino acid sequences are provided in the sequence listing as SEQ ID NO:45-57, respectively. Disulfide stabilized versions of these variant VH regions have the amino acid sequences SEQ ID NO:89-101, respectively. Variant CDR sequences comprised in these variant VH regions are listed as SEQ ID NO:16-28, respectively. The resulting variant VL regions were denoted LC1-LC6 and their amino acid sequences are provided in the sequence listing as SEQ ID NO:59-64, respectively. Disulfide stabilized versions of these variant VL regions have the amino acid sequences SEQ ID NO: 106-111, respectively. Variant CDR sequences comprised in these variant VL regions are listed as SEQ ID NO:29-33, respectively. Table 9 below provides a summary of the specific mutations in each of the alanine variants.

TABLE 9
Alanine substitution variants of VH and VL of h26D3

| Variant | Mutation | Variant | Mutation | Variant | Mutation |
|---|---|---|---|---|---|
| LC1 | Y31A | HC1 | D31A | HC8 | Y60A |
| LC2 | Y38A | HC2 | Y32A | HC9 | Y101A |
| LC3 | Y55A | HC3 | D35A | HC10 | Y106A |
| LC4 | W56A | HC4 | D50A | HC11 | Y107A |
| LC5 | Y97A | HC5 | D54A | HC12 | D111A |
| LC6 | Y100A | HC6 | Y55A | HC13 | Y112A |
| | | HC7 | D56A | | |

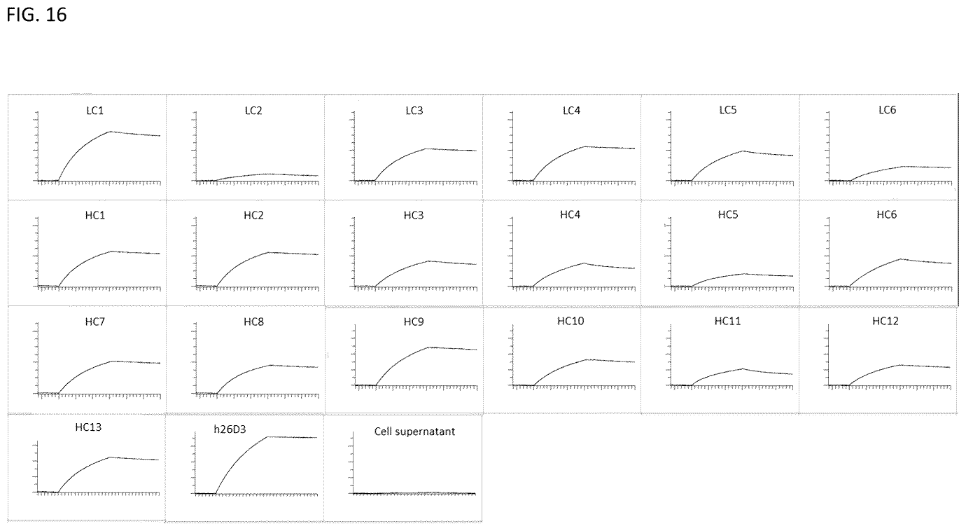

The generated alanine variants were expressed as single mutant, His-tagged Fabs by transient transfection of Chinese hamster ovary cells (ExpiCHO; Thermo Fisher Scientific) according to the manufacturer's instructions. Clarified media, into which the Fabs had been secreted, was used to assess binding to hTfR1 by BLI (Octet RED384, ForteBio). The expressed Fabs were loaded from the cell supernatants onto anti-Fab biosensors during 240 s. Thereafter, association of ectodomain of hTfR1 (SEQ ID NO:74), diluted to 3.75 μg/ml in 1x Kinetics buffer (ForteBio), to the loaded sensors was measured for 300 s, followed by dissociation for 300 s. All variants were confirmed to bind hTfR1 but were affected to different extent (FIG. 16).

Variants showing affected binding to hTfR1 in the screen were selected for further characterization. In addition, double mutants were generated by combining heavy and light chains with alanine substitutions. Table 10 below provides a summary of the specific mutations in each of the alanine variants that were selected.

TABLE 10
Variants of VH and VL of h26D3 selected
for further characterization

| Variant | Mutation VL | Mutation VH |
|---|---|---|
| LC1 | Y31A | None |
| LC5 | Y97A | None |
| LC6 | Y100A | None |
| HC2 | None | Y32A |
| HC3 | None | D35A |
| HC4 | None | D50A |
| HC5 | None | D54A |
| HC6 | None | Y55A |
| HC8 | None | Y60A |
| HC10 | None | Y106A |
| HC11 | None | Y107A |
| HC3/LC1 | Y31A | D35A |
| HC3/LC5 | Y97A | D35A |
| HC6/LC1 | Y31A | Y55A |
| HC6/LC5 | Y97A | Y55A |
| HC8/LC1 | Y31A | Y60A |
| HC8/LC5 | Y97A | Y60A |
| HC10/LC1 | Y31A | Y106A |
| HC10/LC5 | Y97A | Y106A |

The selected variants were expressed as His-tagged Fabs by transient transfection of Chinese hamster ovary cells (ExpiCHO; Thermo Fisher Scientific) according to the manufacturer's instructions. The Fabs were purified at small scale with HisPur™ Ni-NTA Magnetic Beads (Thermo Scientific) according to the manufacturer's instructions followed by buffer exchange into DPBS pH 7.4. Selected variants were also purified at a larger scale by application on a HisTrap Excel column (Cytiva), which was washed with 20 mM Tris, 200 mM NaCl and 5 mM imidazole. The proteins were eluted with 20 mM Tris, 200 mM NaCl and 500 mM imidazole, followed by buffer exchange to DPBS pH 7.4 using a HiPrep 26/10 Desalting column (Cytiva). The proteins were concentrated using an Amicon Ultra centrifugal concentrator (30 MWCO; Millipore). Selected variants were further polished by size exclusion chromatography (SEC; HiLoad 26/600 Superdex 200; Cytiva) in DPBS pH 7.4. Analytical characterization of the protein was done by UV protein determination, SDS-PAGE and HPLC-SEC.

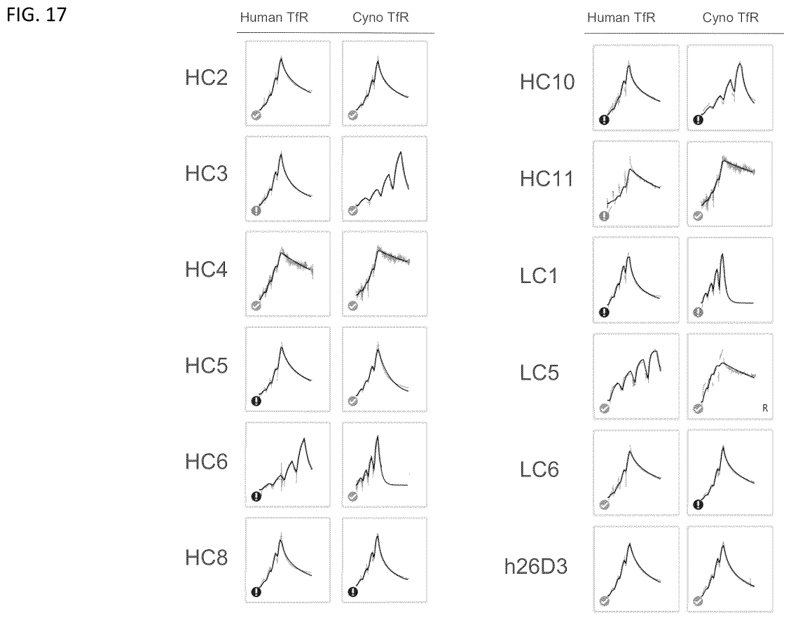

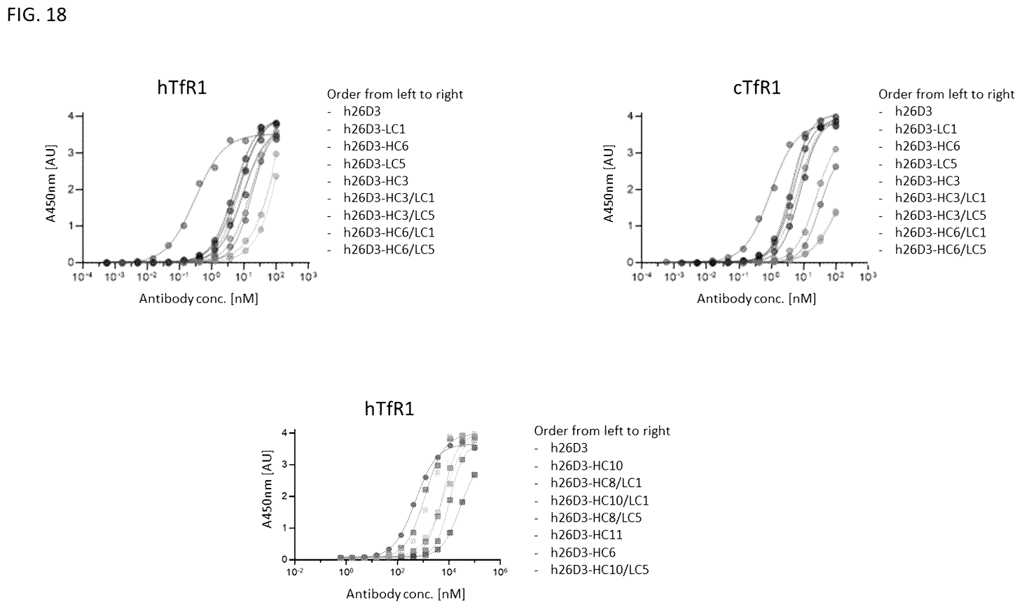

Binding of the purified Fabs to human and cynomolgus TfR1 was evaluated using either SPR (FIG. 17) or indirect ELISA (FIG. 18). For SPR, a Biacore 8K instrument (Cytiva) was used. 1 μg/ml of hTfR1 (SEQ ID NO:86) or cTfR1 (SEQ ID NO: 87) was immobilized on a Cm5 sensor chip (Cytiva, #BR100399) using the amine coupling kit type 2 (Cytiva, #BR100633) according to the manufacturer's instruction. The Fabs were injected over the chip using a 2-fold dilution series in four steps starting at 100 nM. The interaction was measured using the single cycle kinetics method with a contact time of 120 s at a flow rate of 30 μl/min followed by a dissociation time of 1000 s. Regeneration of the surface between cycles was done by injecting 3M MgCl₂. The binding data was fitted to a 1:1 interaction model. The Fabs were diluted in HBS-EP+ (Cytiva, #BR100669). Experiments were performed at 25° C. The results are shown in FIG. 17, and the calculated K_{D} values are given in Table 11 below.

TABLE 11
SPR analysis of variant h26D3 Fabs vs. hTfR1 and cTfR1

| Variant | | |
|---|---|---|
| h26D3 | hTfR1 | cTfR1 |
| Fab | K_{D} (nM) | K_{D} (nM) |
| HC2 | 8.7 | 12 |
| HC3 | 16 | 168 |
| HC4 | 5.1 | 8 |
| HC5 | 20 | 39 |
| HC6 | 156 | 164 |
| HC8 | 10 | 19 |
| HC10 | 9.2 | 4.6 |
| HC11 | 388 | 2.7 |
| LC1 | 14 | 70 |
| LC5 | 240 | ND |
| LC6 | 8.5 | 9.4 |
| h26D3 | 10 | 12 |

For the indirect ELISA, half area 96-well plates (Corning, #3690) were coated with 1 μg/ml recombinant ectodomain of hTfR1 (SEQ ID NO:74) in PBS overnight at 4° C. The coated plates were blocked using Pierce protein-free blocking solution (Thermo Fisher Scientific, #37572) for 1 h at room temperature with shaking and washed four times in PBS containing 0.1% TWEEN-20. Serial dilutions (1:3) of various expressed constructs in incubation buffer (1% BSA, 0.1% TWEEN-20 in PBS) were incubated for 1 h at room temperature. Following the four wash steps, bound test constructs were detected by addition of anti-human-IgG F (ab′) 2-HRP antibody (Jackson Immuno Research, #109-036-003) at 1:5000 dilution in incubation buffer (1 h, room temperature). Following four wash steps, K-Blue® Aqueous TMB substrate (Neogen, #331177) was added to the wells for 15 min at room temperature before the reaction was stopped with 1:1 dilution of 0.5 M H₂SO₄. The optical density at 450 nm was recorded (Spark, Tecan) and background signal was subtracted before analysis. The obtained results are shown in FIG. 18.

Based on the Biacore and ELISA measurements, several variants were identified within a wide range of affinities for human TfR1. Many variants exhibited a retained cross-reactivity to cynomolgus TfR1.

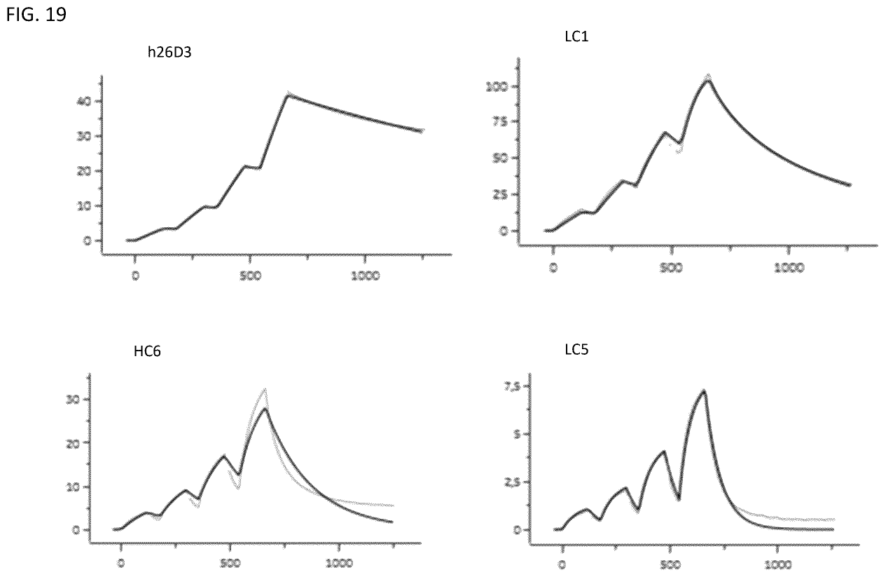

Finally, selected variants were reformatted to scFv and used in the context of the bispecific binding molecule format disclosed in WO2022/258841. Bispecific binding molecules comprising scFv modules constructed from h26D3 and selected alanine mutants were expressed in ExpiCHO cells as described above. Filtered supernatants were applied to a MabSelect SuRe column (Cytiva) which was subsequently washed with DPBS pH 7.4. Expressed binding molecules were eluted by application of 0.7% HAc pH 2.5, followed by immediate neutralization of the sample to pH 7.5. Purified samples were polished further by subjecting them to size exclusion chromatography (SEC; HiLoad 26/600 Superdex 200; Cytiva) in DPBS pH 7.4. The purified constructs were concentrated using centrifugal concentrators Amicon Ultra (30 MWCO, Millipore). Each purified expressed construct was characterized using SDS-PAGE, size-exclusion chromatography (Superdex 200 Increase 3.2/300; Cytiva) and UV protein determination. Binding to hTfR1 was evaluated using SPR as described above with adjustments of the concentration interval depending on the variant. As shown in FIG. 19 and in Table 12 below, the different tested variants exhibited a range of affinities for the hTfR1 target.

TABLE 12
SPR analysis of variant h26D3 scFv in bispecific format vs. hTfR1

| Variant | K_{D} vs hTfR1 (nM) |
|---|---|
| h26D3 | ~10 |
| LC1 | ~40 |
| HC6 | ~100-200 |
| LC5 | ~400-500 |

### Example 10

### Design, Production and Preparative SEC of Disulfide-Stabilized hTfR1 Binding Molecules

A panel of hTfR1 binding molecules in the scFv format were designed, produced and purified. The designed hTfR1 binding scFv molecules are listed in Table 13.

TABLE 13
hTfR1 binding scFv molecules and their amino acid sequences

| Designation | Amino acid sequence |
|---|---|
| h26D3-HC6 | SEQ ID NO: 115 |
| h26D3-HC6_DS | SEQ ID NO: 116 |
| h26D3-HC6, VL-first | SEQ ID NO: 117 |
| h26D3-HC6_DS, VL-first | SEQ ID NO: 118 |
| h26D3-LC1 | SEQ ID NO: 119 |
| h26D3-LC1_DS, VL-first | SEQ ID NO: 120 |
| h26D3wt | SEQ ID NO: 80 |
| h26D3wt_DS, VL-first | SEQ ID NO: 121 |

The scFv variants whose respective designation includes the “_DS” suffix all comprise two mutations which introduce cysteine residues at position 44 of the VH domain and at position 106 of the VL domain of the respective starting sequences. It is contemplated that these introduced cysteine residues cause the formation of a stabilizing disulfide bond between the VH and VL domains.

The test items were produced as His₆ tagged scFv constructs with the His₆ tag spaced from the remainder of the scFv by a flexible (G4S)₄ linker (combined tag sequence given by SEQ ID NO:122), by transient transfection of CHO cells in 400 ml culture volume per scFv. One of the test items, “h26D3-wt_DS, VL-first”, was also produced with both an His₆ tag and an Avi tag (combined tag sequence given by SEQ ID NO: 123) for site directed in vivo biotinylation, and was expressed in 1| culture volume.

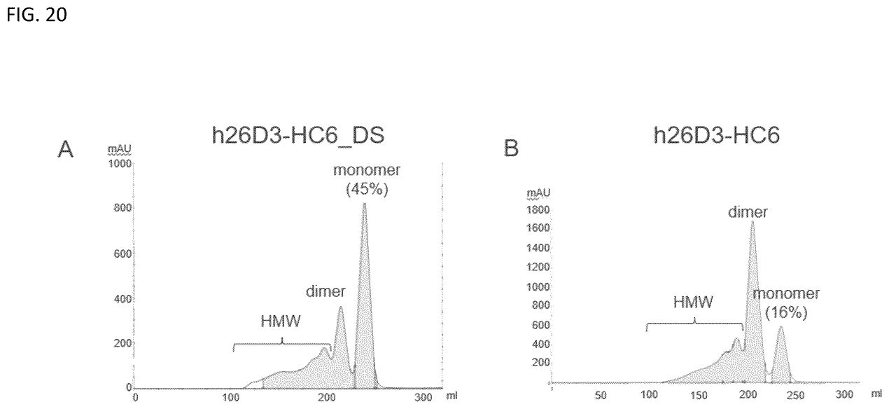

For purification, all scFv proteins were recovered by immobilized metal ion affinity chromatography (IMAC) purification from clarified cell supernatants. For IMAC, supernatants were loaded on a HisTrap excel 5 ml column (Cytiva) and unbound material washed out with wash buffer (PBS, 350 mM NaCl and 10 mM imidazole). Bound scFv was then eluted in elution buffer (PBS, 350 mM NaCl, 0.5 M imidazole, pH 7.5). Next, the eluted proteins were passed over a preparative SEC column (HiLoad 26/600 Superdex 200 μg; Cytiva) with PBS, pH 7.4 as running buffer. SEC fractions containing monomeric scFv were collected and brought to 1 mg/ml final concentration in PBS, pH 7.4. Representative chromatograms from this preparative SEC are shown in FIG. 20 for h26D3-HC6_DS (A) and h26D3-HC6 (B), and show that the scFv molecules are recovered with different degrees of aggregated forms during the initial IMAC purification. For h26D3-HC6_DS (A), 45% of the material elutes in the main peak and contain the monomeric, desired scFv. This is in contrast to h26D3-HC6 (B), for which the distribution between dimer and monomer is the opposite, showing scFv dimer in the main peak, and only 16% of the material in the monomer peak. The size distribution of higher molecular weight (HMW) species is similar for both constructs.

### Example 11

### Analytical SEC of Disulfide-Stabilized hTfR1 Binding Molecules

Following three freeze/thaw cycles between room temperature and −80° C., 1 μg of each scFv variant produced in Example 10 was injected to a SEC column (Waters BioSuite 250 UHR SEC 4 μm, 4.6×300 mm). Analyses were done with a running buffer of 0.2 M potassium phosphate, 0.25 M KCl, pH 6.2 at a flow rate of 0.35 ml/min.

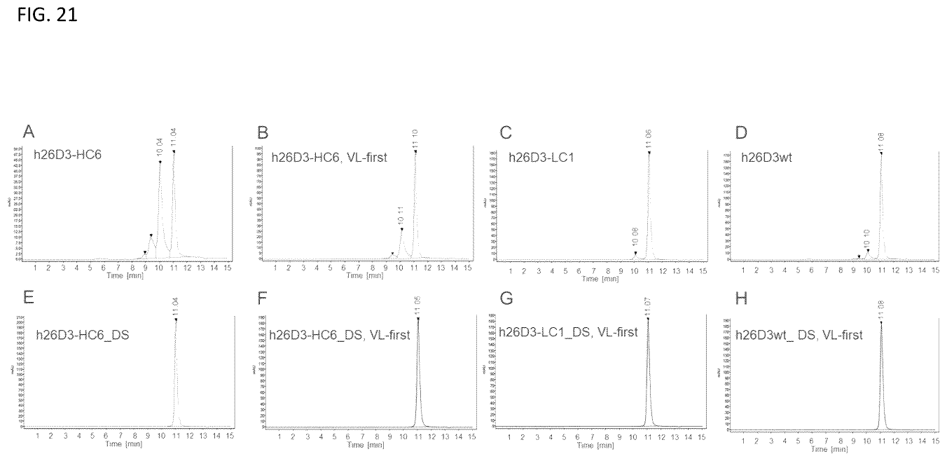

The results of the analytical SEC experiment are shown in FIG. 21 and Table 14. The monomeric form of all scFv samples have a retention time of 11 min (FIG. 21A-H). In scFv molecules lacking the DS mutations, additional peaks, corresponding to multimerized forms of scFv are detected (FIG. 21A-D). In all samples with DS mutations, 100% of the respective molecule migrate at 11 min as monomeric scFv (FIG. 21E-H). The percentage distribution of integrated peak areas from the analytical SEC samples are listed in Table 14. Again, for the four samples with stabilizing DS mutations, 100% of injected proteins are detected in the monomer peak, whereas additional peaks of multimeric forms are detected for corresponding samples without DS mutations.

TABLE 14
Distribution of peak areas from SEC chromatograms

| | Chromatogram | Monomer | Dimer | Tetramer | Hexamer |
|---|---|---|---|---|---|
| Molecule | in FIG. 21 | (%) | (%) | (%) | (%) |
| h26D3-HC6 | A | 38 | 47 | 13 | 2 |
| h26D3-HC6, VL-first | B | 73 | 23 | 4 | |
| h26D3-LC1 | C | 94 | 6 | | |
| h26D3wt | D | 90 | 9 | 1 | |
| h26D3-HC6_DS | E | 100 | | | |
| h26D3-HC6_DS, VL-first | F | 100 | | | |
| h26D3-LC1_DS, VL-first | G | 100 | | | |
| h26D3wt_DS, VL-first | H | 100 | | | |

### Example 12

### Thermal Stability of Disulfide-Stabilized hTfR1 Binding Molecules

Monomer stability of scFv samples was evaluated by HPLC SEC analysis. The panel of scFv molecules produced and studied in Examples 10-11, purified and stored in PBS, were subjected to temperature hold for one, two or four weeks at temperatures 4° C., 40° C. and frozen at −80° C., except for h26D3-HC6_DS, VL-first and h26D3-LC1, VL-first, which were held frozen at −70° C. and−75° C. respectively. At each timepoint, samples of each variant from each temperature were analyzed by HPLC-SEC as described in Example 11. At the initiation of the study, frozen samples were thawed and analyzed, and are denoted TO.

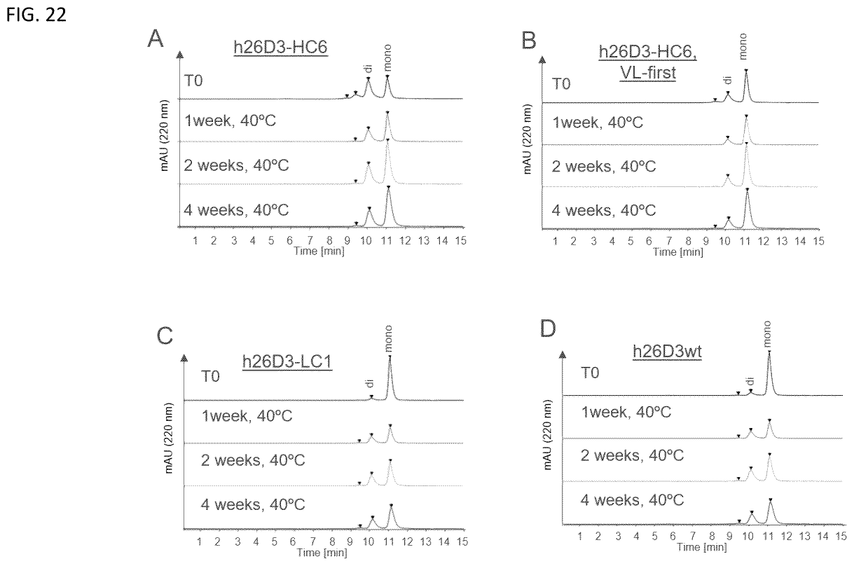

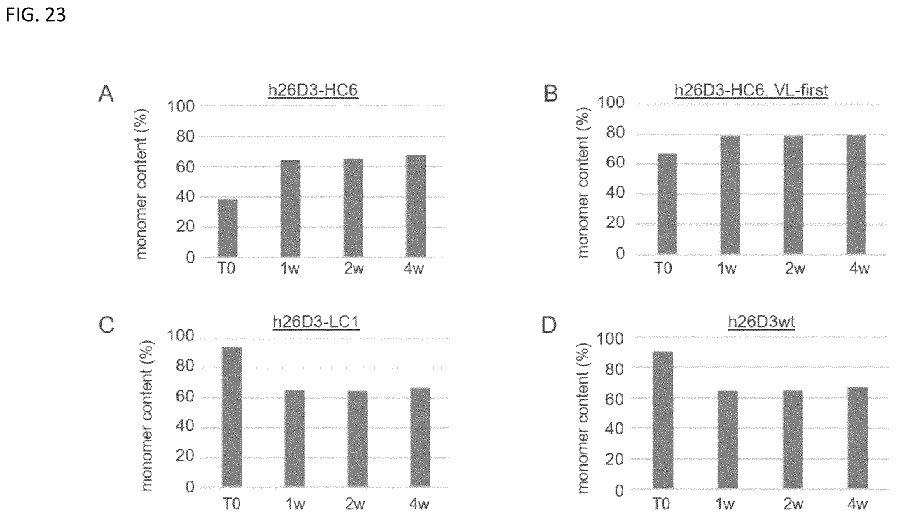

The results for scFv molecules without DS mutations after storage at 40° C. for 1-4 weeks are shown in FIGS. 22 and 23. All samples were isolated as pure monomers in the preceding preparative SEC purification described in Example 10. However, analytical SEC revealed that all samples contain both monomer (retention at approximately 11 min) and dimer (retention at approximately 10 min) forms already at the initial time point TO (FIG. 22A-D). The share of dimers is the most significant for h26D3-HC6 (FIG. 22A) and h26D3-HC6, VL-first (FIG. 22B), while the majority of scFv molecules are monomeric at TO for h26D3-LC1 (FIG. 22C) and h26D3 wt (FIG. 22D). The chromatograms show that the distribution between monomeric and dimeric forms shifts gradually during the study. In samples from 4 weeks, the monomer/dimer distributions are more similar between the different molecules as compared to the corresponding distributions at TO (FIG. 22A-D). Multimers are observed for all scFv molecules as a minor peak with a retention time between 9-10 min (FIG. 22A-D). The results indicate that, despite having been isolated in the pure monomeric form, scFv molecules without the stabilizing DS mutations form multimers during storage.

The same pattern is shown by the percentage proportions of monomeric scFv molecules exhibited in FIG. 23. The molecules h26D3-HC6 (FIG. 23A) and h26D3-HC6, VL-first (FIG. 23B) have lower proportions of monomeric scFv at TO (reference sample kept at −80° C.). The degree of monomer increases for these molecules in samples kept at 40° C. for 1-4 weeks (FIG. 23A-B). The opposite is seen for h26D3-LC1 (FIG. 23C) and h26D3 wt (FIG. 23D), where monomer content is high at TO and then decreases during storage at 40° C. for 1-4 weeks. The observation indicates that scFv molecules without DS mutations reach an equilibrium between monomeric and dimeric states during storage. For h26D3-HC6 and h26D3-HC6 VL-first, the monomer content increased over the course of the study, while for h26D3-LC1 and h26D3 wt, the monomer content decreased as compared to T0.

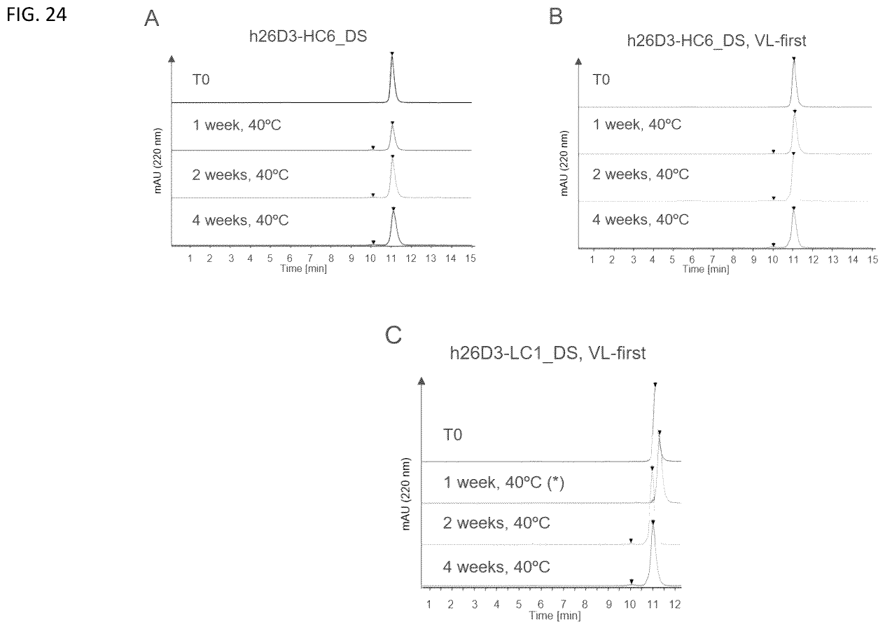

Corresponding chromatograms for scFv molecules with DS mutations after storage at 40° C. for 1-4 weeks are shown in FIG. 24. As seen here, the molecules with introduced DS mutations are highly stable as monomers over the studied period, with uniform peaks of monomeric scFv detected at a retention time just above 11 min for all the variants (FIG. 24A-C). Only in chromatograms from samples incubated for 4 weeks at 40° C. (FIG. 24C), a very small peak is observed at 10 min retention time. The results demonstrate that the monomeric state of DS-stabilized scFv molecules is highly stable also at an extended storage time at 40° C.

As can be seen in Table 15 below, the high stability of the scFv variants with DS mutations as compared to corresponding variants without DS mutations was also seen for samples kept at 4° C. or frozen.

TABLE 15
Percentage of monomer forms of scFv samples

| | h26D3-HC6 | | | | h26D3-HC6, VL-first | | | |
|---|---|---|---|---|---|---|---|---|
| Timepoint | Temp | | Retention time | Monomer % | Temp | | Retention time | Monomer % |
| T0 | | | 11.04 | 38.58 | | | 11.099 | 66.86 |
| 1 w | +4° | C. | 11.048 | 47.16 | +4° | C. | 11.104 | 74.81 |
| 2 w | +4° | C. | 11.047 | 50.75 | +4° | C. | 11.107 | 72.22 |
| 4 w | +4° | C. | 11.087 | 54.25 | +4° | C. | 11.148 | 70.69 |
| 1 w | +40° | C. | 11.048 | 64.23 | +40° | C. | 11.106 | 78.68 |
| 2 w | +40° | C. | 11.055 | 64.95 | +40° | C. | 11.114 | 78.72 |
| 4 w | +40° | C. | 11.099 | 67.56 | +40° | C. | 11.156 | 79.02 |
| 1 w | −80° | C. | 11.055 | 41.54 | −80° | C. | 11.107 | 52.6 |
| 2 w | −80° | C. | 11.053 | 45.24 | −80° | C. | 11.114 | 49.96 |
| 4 w | −80° | C. | 11.089 | 45.19 | −80° | C. | 11.155 | 56.69 |
| | h26D3-LC1 | | | | h26D3wt | | | |
| Timepoint | Temp | | Retention time | Monomer % | Temp | | Retention time | Monomer % |
| T0 | | | 11.065 | 93.96 | | | 11.079 | 90.26 |
| 1 w | +4° | C. | 11.079 | 88.36 | +4° | C. | 11.092 | 82.78 |
| 2 w | +4° | C. | 11.081 | 83.04 | +4° | C. | 11.093 | 76.23 |
| 4 w | +4° | C. | 11.126 | 75.98 | +4° | C. | 11.139 | 68.6 |
| 1 w | +40° | C. | 11.08 | 65.18 | +40° | C. | 11.094 | 64.55 |
| 2 w | +40° | C. | 11.087 | 64.61 | +40° | C. | 11.101 | 64.72 |
| 4 w | +40° | C. | 11.133 | 66.67 | +40° | C. | 11.145 | 66.8 |
| 1 w | −80° | C. | 11.078 | 87.63 | −80° | C. | 11.091 | 89.57 |
| 2 w | −80° | C. | 11.084 | 88.89 | −80° | C. | 11.095 | 86.32 |
| 4 w | −80° | C. | 11.128 | 91.28 | −80° | C. | 11.141 | 78.9 |

| | h26D3-HC6_DS | | | | h26D3-HC6_DS, VL-first | | | | h26D3-LC1_DS, VL-first | | | |
|---|---|---|---|---|---|---|---|---|---|---|---|---|
| | | | Retention | Monomer | | | Retention | Monomer | | | Retention | Monomer |
| Timepoint | Temp | | time | % | Temp | | time | % | Temp | | time | % |
| T0 | | | 11.034 | 100 | | | | | | | 11.151 | 99.47 |
| 1 w | +4° | C. | 11.046 | 100 | +4° | C. | 11.095 | 100 | +4° | C. | 11.342 | 100 |
| 2 w | +4° | C. | 11.05 | 100 | +4° | C. | 11.021 | 100 | +4° | C. | 10.997 | 98.63 |
| 4 w | +4° | C. | 11.092 | 99.36 | +4° | C. | 11.029 | 100 | +4° | C. | 11.03 | 99.19 |
| 1 w | +40° | C. | 11.048 | 99.42 | +40° | C. | 11.095 | 100 | +40° | C. | 11.329 | 98.91 |
| 2 w | +40° | C. | 11.056 | 98.83 | +40° | C. | 11.027 | 99.2 | +40° | C. | 11.008 | 97.69 |
| 4 w | +40° | C. | 11.098 | 98.24 | +40° | C. | 11.040 | 98.36 | +40° | C. | 11.052 | 97.34 |
| 1 w | −80° | C. | 11.045 | 100 | −70° | C. | 11.1 | 100 | −75° | C. | 11.364 | 100 |
| 2 w | −80° | C. | 11.051 | 100 | −70° | C. | 11.024 | 100 | −75° | C. | 10.998 | 98.82 |
| 4 w | −80° | C. | 11.091 | 99.54 | −70° | C. | 11.032 | 100 | −75° | C. | 11.029 | 99.43 |

### Example 13

Serum stability of disulfide-stabilized hTfR1 binding molecules Serum stability is a critical attribute for antibodies and different fragments such as scFv-containing biotherapeutics (Worn and Pluckthun (2001), J Mol Biol 305 (5): 989-1010; Austerberry et al (2017), Eur J Pharm Biopharm 115:18-30; Willuda et al (1999), Cancer Res 59:5758-67). In order to assess the stability in serum of scFv molecules with DS mutations, the variant h26D3 wt_DS, VL-first expressed with His₆ and Avi tags (see Example 10) was incubated in mouse serum (Capricon, MOU-1B) and 1xPBS (#09-9400-100, Medicago AB) respectively at both 4° C. and 37° C. using a thermal mixer (Eppendorf ThermoMixer C, Eppendorf). After 48 h incubation, binding of the scFv to hTfR1 was evaluated by ELISA. In brief, a half area 96-well plate (#3690, Corning) was coated overnight at 4° C. with hTfR1 diluted in 1xPBS, followed by blocking with Pierce Protein-Free Blocking Buffer (#37572, Thermo Fisher Scientific) for 1 h at room temperature (RT) with shaking. h26D3 wt_DS, VL-first was diluted or serially diluted in mouse serum or ELISA incubation buffer (EIB): 1xPBS-0.05% Tween20, 0.1% BSA (PBS-T, #09-9410-100, Medicago AB, A7030-100G, Sigma-Aldrich), added to plate and incubated for 2 h at 4-8° C. with shaking. Bound biotinylated scFv was detected using streptavidin-horseradish-peroxidase (#3310-9-100, Mabtech) in EIB for 1 h at RT with shaking, followed by TMB (#331177, Neogen). The reaction was stopped by 1:1 addition of 0.5 M sulfuric acid (#35354-1L, Honeywell). Optical density at 450 nm was obtained using a microplate reader (Spark, Tecan) and the collected data was plotted using GraphPad Prism software (GraphPad Software Inc). Serum stability of the scFv is displayed as % binding to hTfR1 and determined using the following equation:

$$Serum stability = ( ELISA OD 450 at 37 ° C . ) / ( ELISA OD 450 at 4 ° C . ) \times 100 \%$$

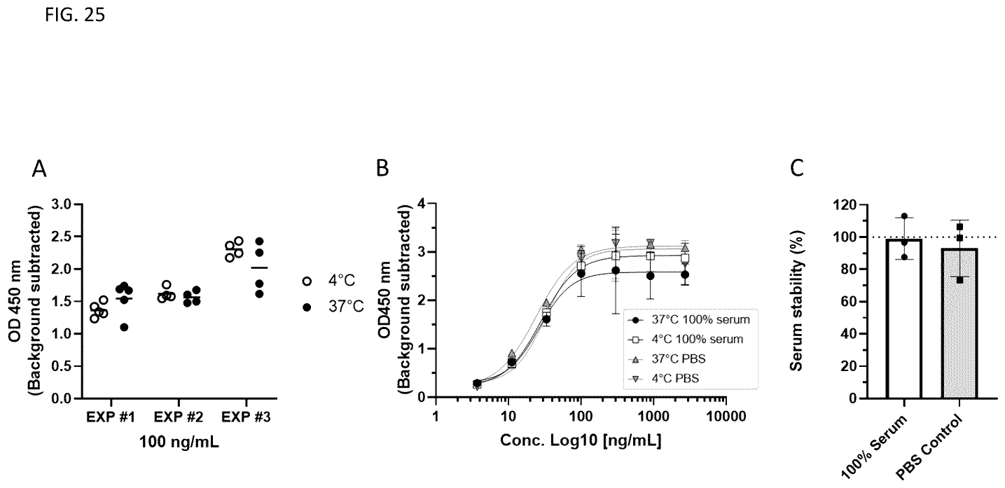

The results are shown in FIG. 25, and demonstrate stability and a highly retained hTfR1 binding ability of the tested scFv variant after incubation in mouse serum for 48 h at the tested temperatures. The results can be compared to other published scFv stability data in mouse serum (Liu et al (2022), mAbs 14:1, 2073632).

### Example 14

### Dynamic Light Scattering Analysis of Disulfide-Stabilized hTfR1 Binding Molecules

Dynamic light scattering (DLS) analysis of scFv variants h26D3-HC6_DS, VL-first; h26D3-HC6 and h26D3-HC6_DS (see Example 10) was performed at 25° C. or 20° C. using an Uncle instrument (Unchained Labs). Average hydrodynamic diameter and polydispersity index (PDI) were calculated from analyses run in triplicates h26D3-HC6_DS, VL-first) or duplicates (h26D3-HC6 and h26D3-HC6_DS). Samples were diluted to 1 mg/ml in PBS prior to analysis.

The results are shown in Table 16. The scFv variant without DS mutations (h26D3-HC6) exhibits a larger average hydrodynamic diameter than either of the two variants with DS mutations (h26D3-HC6_DS, VL-first and h26D3-HC6_DS). The results are expected from the high monomeric content for DS stabilized scFv as shown by analytical SEC (FIG. 21 and Table 14), and agree with other reported DLS analyses of scFv (Morioka et al (2019), Molecules 24 (14): 2620). The observed PDI values are around 0.1 in all variants, indicating monodisperse (PDI<0.1) or a low degree of polydispersity (PDI 0.1-0.2).

TABLE 16
Dynamic light scattering analysis of scFv variants

| | Average hydrodynamic | |
|---|---|---|
| Molecule | diameter (nm) | PDI |
| h26D3-HC6_DS, VL-first | 4.8 | 0.13 |
| h26D3-HC6 | 6.9 | 0.08 |
| h26D3-HC6_DS | 4.6 | 0.09 |

### Example 15

Surface plasmon resonance analysis of disulfide-stabilized hTfR1 binding molecules Binding to hTfR1 of six different purified scFv variants from Example 10 was evaluated by surface plasmon resonance (SPR) using Biacore 8K (Cytiva). 30 μg/ml of DYKDDDDK Tag mAb FG4R (Thermo Fisher Scientific, MA1-91878) in 10 mM acetate buffer, pH 4.5 was immobilized on a CM5 sensor chip (Cytiva, #BR100399) using the amine coupling kit type 2 (Cytiva, #BR100633) according to the manufacturer's instruction, resulting in immobilization levels of approximately 10000 RU. hTfR1 was captured via an N-terminal DYKDDDDK tag. To this end, the receptor was injected over the chip with a contact time of 60 s at a flow rate of 10 μl/min. Each of the six scFv variants h26D3-HC6; h26D3-HC6, VL-first; h26D3-LC1; h26D3-HC6_DS; h26D3-HC6_DS, VL-first and h26D3-LC1_DS, VL-first were injected over the chip using a 3-fold dilution series in five steps starting at 700 nM or 400 nM. Interaction was measured using the single cycle kinetics method with a contact time of 120 s at a flow rate of 30 μl/min followed by a dissociation time of 600 s. Regeneration of the surface between cycles was done by injecting 10 mM glycine-HCl pH 1.7 with a contact time of 30 s and a flow rate of 30 μl/min. The binding modules were diluted in HBS-EP+ (Cytiva, #BR100669). Experiments were performed at 25° C.

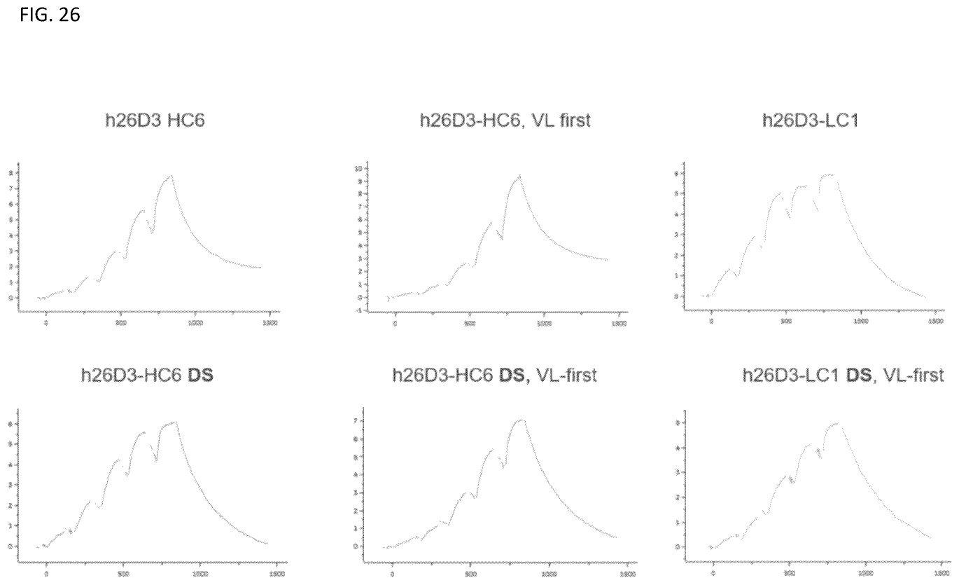

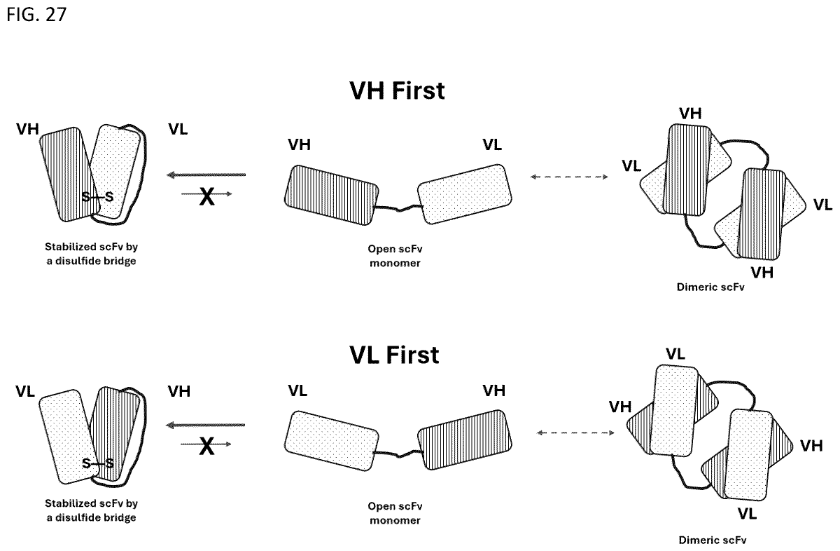

The resulting binding curves are shown in FIG. 26. As can be seen from the SPR diagrams, introduction of stabilizing DS mutations does not disrupt the binding of hTfR1 by the scFv variants. It can also be seen that the scFv variants without DS mutations dissociate from the hTfR1 antigen in a biphasic manner, which may be due to an element of avidity interaction observed for dimeric forms present in the samples.

### Example 16

### Design, Production and SEC Analysis of Additional Stabilized hTfR1 Binding Molecules

In the single-chain variable fragment (scFv) format, hydrophobic patches within the former V/C interface of the full-length antibody become exposed (Nieba et al (1997), Prot Eng 10 (4): 435-444). These newly accessible hydrophobic areas, particularly within the VH domain, can serve as binding sites for preexisting anti-drug antibodies (PE-ADA) when the scFv, or construct comprising it, is administered to a subject (Holland et al (2013), J Clin Immunol 33:1192-1203). Previous work has shown that mutation of certain hydrophobic residues within the former V/C interface of the VH domain to more hydrophilic residues leads to a reduction of such potential PE-ADA reactivity (Johansson et al (2023), mAbs 15 (1): 2215887).

Eleven variants of the disulfide-stabilized VH domain of h26D3-HC6 were designed, containing either single or double substitutions of hydrophobic amino acid residues to more hydrophilic and/or less bulky amino acids residues. The variants are listed in Table 17. All VH domain variants were expressed in the form of scFv domains having the VL first orientation. A flexible (G4S)₄ linker connects the VL domain to the VH domain within each scFv. For purification purposes, a His₆ tag and (G₄S)₄ linker was added to the N-terminus of the VL domain (combined tag sequence given by SEQ ID NO:122).

TABLE 17
VH variants of the hTfR1 binding scFv
molecule h26D3-HC6_DS, VL-first

| | VH AA sequence | scFv AA sequence |
|---|---|---|
| Designation | (SEQ ID NO:) | (SEQ ID NO:) |
| h26D3 HC6 DS S122K | 146 | 305 |
| h26D3 HC6 DS T120K | 163 | 306 |
| h26D3 HC6 DS L118S | 296 | 314 |
| h26D3 HC6 DS T91S S122K | 130 | 307 |
| h26D3 HC6 DS T91S T120K | 197 | 308 |
| h26D3 HC6 DS T91A | 247 | 311 |
| h26D3 HC6 DS S16E L118S | 264 | 312 |
| h26D3 HC6 DS S16E | 279 | 313 |
| h26D3 HC6 DS V11R T120K | 180 | 309 |
| h26D3 HC6 DS V11R T91A | 213 | 310 |
| h26D3 HC6 DS V11R | 230 | 315 |

These eleven scFv variants were produced in CHO cells and purified with IMAC plus preparative SEC as described in Example 10. The SEC fractions containing monomeric scFv were collected, sterile filtered and brought to 1 mg/ml final concentration in PBS, pH 7.4. The recovered molecules were analysed by CE-SDS, reducing and non-reducing, by loading 2.5 μg of respective molecule on a LabChip (GXII Touch™ HT Chip, PerkinElmer). Samples were prepared with the ProteinEXact Assay Reagent Kit (PerkinElmer). Instrument Assay used: LabChip GXII Touch HT Protein Characterization System (Perkin Elmer). Sample treatment: Before loading. samples were treated with DTT for reduced conditions, and both non-reduced and reduced samples were heated at 70° C. for 10 min. The results showed bands of expected size and high purity for all molecules.

Each scFv variant produced was then injected onto an SEC column and analyzed as described in Example 11. The results of the analytical SEC experiment are shown in Table 18 and demonstrate that all variants of the molecule were isolated with a high monomer content, similar to the DS-stabilized scFv molecules produced in Example 11.

TABLE 18
Results from aSEC of hTfR1 binding scFv variants

| | Main peak | |
|---|---|---|
| | retention time | Main peak area |
| scFv variant | (min) | (%) |
| h26D3 HC6 DS S122K | 10.905 | 99.85 |
| h26D3 HC6 DS T120K | 10.909 | 99.85 |
| h26D3 HC6 DS L118S | 10.910 | 99.85 |
| h26D3 HC6 DS T91S S122K | 10.910 | 99.86 |
| h26D3 HC6 DS T91S T120K | 10.914 | 99.87 |
| h26D3 HC6 DS T91A | 10.905 | 99.88 |
| h26D3 HC6 DS S16E L118S | 10.903 | 100.00 |
| h26D3 HC6 DS S16E | 10.902 | 100.00 |
| h26D3 HC6 DS V11R T120K | 10.922 | 99.84 |
| h26D3 HC6 DS V11R T91A | 10.919 | 99.88 |
| h26D3 HC6 DS V11R | 10.923 | 99.89 |

Based on the successful introduction of single and double mutations into the VH domain of stabilized h26D3 HC6 as evidenced above, the same mutations may also be introduced in any other VH domain of the disclosure. Introducing each of the eleven mutation patterns into each VH domain of the stabilized, murine and humanized, hTfR1 binding molecules disclosed herein, gives rise to VH domains having the sequences SEQ ID NO:124-304.

### Example 17

### Thermal Stability of Additional Variants of hTfR1 Binding Molecules

From the production and analysis of hTfR binding molecules in Example 16, six variant scFv: s (SEQ ID NO:305-310) were selected and subjected to a thermal stability study. Monomer stability of scFv samples was evaluated by HPLC SEC analysis. The scFv molecules, purified and stored in PBS, were subjected to temperature hold for one, two or four weeks at temperatures 4° C., 25° C., 40° C. and frozen at −75° C. At each timepoint, samples of each variant from each temperature were analyzed by HPLC-SEC as described in Example 16. At the initiation of the study, frozen samples were thawed and analyzed, and denoted “TO”. In addition, for each of the six molecules a sample was subjected to freeze-thaw stress. Molecules were subjected to 3x freeze-thaw cycles. Freezing was done at −75° C. for at least 16 h and thawing was done at ambient temperature until complete thawing of the sample (up to 60 min). Samples are denoted FT_3x and were analyzed immediately after the third freeze-thaw cycle completion.

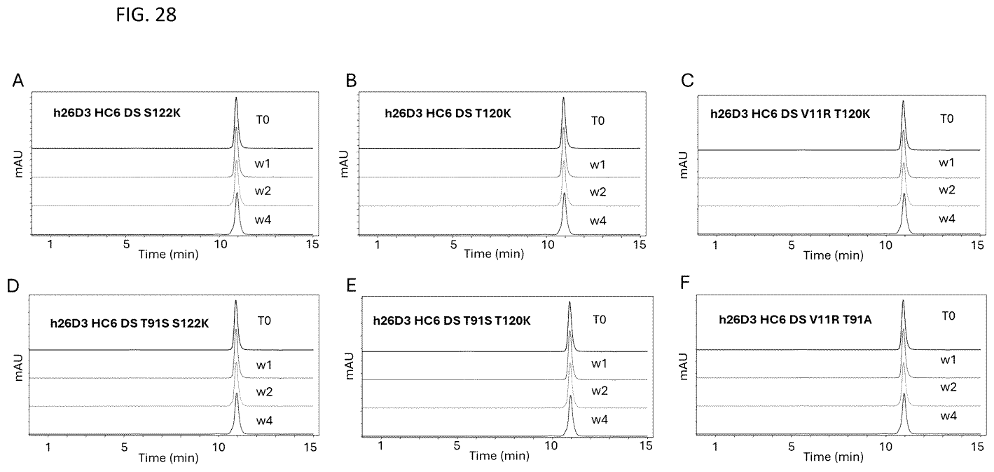

All six scFv molecules were stable monomers at the tested conditions. The main peak area from analytical SEC are shown in Tables 19-24. The results for the six selected scFv molecules from samples collected as TO or after temperature hold at 40° C. for 1-4 weeks respectively are shown as stacked chromatograms in FIG. 28A-F.

TABLE 19
Analytical SEC from thermal stability study on h26D3 HC6 DS S122K

| | | | | aSEC. | Main |
|---|---|---|---|---|---|
| | | | | Main peak | peak |
| | Temp | | Time | retention | area |
| Sample | (° C.) | | (week) | (min) | (%) |
| h26D3 HC6 DS S122K_T0 | −75° | C. | 0 | 10.901 | 99.85 |
| h26D3 HC6 DS S122K_FT_3x | — | | 0 | 10.909 | 99.86 |
| h26D3 HC6 DS S122K_+4 C._w 1 | +4° | C. | 1 | 10.907 | 99.83 |
| h26D3 HC6 DS S122K_+4 C._w 2 | +4° | C. | 2 | 10.919 | 99.82 |
| h26D3 HC6 DS S122K_+4 C._w 4 | +4° | C. | 4 | 10.936 | 99.75 |
| h26D3 HC6 DS S122K_+25 C._w 1 | +25° | C. | 1 | 10.908 | 99.62 |
| h26D3 HC6 DS S122K_+25 C._w 2 | +25° | C. | 2 | 10.919 | 99.64 |
| h26D3 HC6 DS S122K_+25 C._w 4 | +25° | C. | 4 | 10.938 | 99.56 |
| h26D3 HC6 DS S122K_+40 C._w 1 | +40° | C. | 1 | 10.906 | 99.74 |
| h26D3 HC6 DS S122K_+40 C._w 2 | +40° | C. | 2 | 10.922 | 98.90 |
| h26D3 HC6 DS S122K_+40 C._w 4 | +40° | C. | 4 | 10.944 | 93.82 |

TABLE 20
Analytical SEC from thermal stability study on h26D3 HC6 DS T120K

| | | | | aSEC. | Main |
|---|---|---|---|---|---|
| | | | | Main peak | peak |
| | Temp | | Time | retention | area |
| Sample | (° C.) | | (week) | (min) | (%) |
| h26D3 HC6 DS T120K_T0 | −75° | C. | 0 | 10.906 | 99.87 |
| h26D3 HC6 DS T120K_FT_3x | — | | 0 | 10.914 | 99.88 |
| h26D3 HC6 DS T120K_+4 C._w 1 | +4° | C. | 1 | 10.914 | 99.84 |
| h26D3 HC6 DS T120K_+4 C._w 2 | +4° | C. | 2 | 10.924 | 99.78 |
| h26D3 HC6 DS T120K_+4 C._w 4 | +4° | C. | 4 | 10.941 | 99.79 |
| h26D3 HC6 DS T120K_+25 C._w 1 | +25° | C. | 1 | 10.913 | 99.64 |
| h26D3 HC6 DS T120K_+25 C._w 2 | +25° | C. | 2 | 10.923 | 99.63 |
| h26D3 HC6 DS T120K_+25 C._w 4 | +25° | C. | 4 | 10.942 | 99.58 |
| h26D3 HC6 DS T120K_+40 C._w 1 | +40° | C. | 1 | 10.912 | 99.76 |
| h26D3 HC6 DS T120K_+40 C._w 2 | +40° | C. | 2 | 10.927 | 98.89 |
| h26D3 HC6 DS T120K_+40 C._w 4 | +40° | C. | 4 | 10.949 | 94.18 |

TABLE 21
Analytical SEC from thermal stability study on h26D3 HC6 DS T91S S122K

| | | | | aSEC. | Main |
|---|---|---|---|---|---|
| | | | | Main peak | peak |
| | Temp | | Time | retention | area |
| Sample | (° C.) | | (week) | (min) | (%) |
| h26D3 HC6 DS T91S S122K_T0 | −75° | C. | 0 | 10.901 | 99.87 |
| h26D3 HC6 DS T91S S122K_FT_3x | — | | 0 | 10.912 | 99.88 |
| h26D3 HC6 DS T91S S122K_+4 C._w 1 | +4° | C. | 1 | 10.905 | 99.84 |
| h26D3 HC6 DS T91S S122K_+4 C._w 2 | +4° | C. | 2 | 10.919 | 99.75 |
| h26D3 HC6 DS T91S S122K_+4 C._w 4 | +4° | C. | 4 | 10.935 | 99.79 |
| h26D3 HC6 DS T91S S122K_+25 C._w 1 | +25° | C. | 1 | 10.912 | 99.59 |
| h26D3 HC6 DS T91S S122K_+25 C._w 2 | +25° | C. | 2 | 10.920 | 99.58 |
| h26D3 HC6 DS T91S S122K_+25 C._w 4 | +25° | C. | 4 | 10.937 | 99.52 |
| h26D3 HC6 DS T91S S122K_+40 C._w 1 | +40° | C. | 1 | 10.908 | 99.73 |
| h26D3 HC6 DS T91S S122K_+40 C._w 2 | +40° | C. | 2 | 10.923 | 98.94 |
| h26D3 HC6 DS T91S S122K_+40 C._w 4 | +40° | C. | 4 | 10.944 | 93.82 |

TABLE 22
Analytical SEC from thermal stability study on h26D3 HC6 DS T91S T120K

| | | | | aSEC. | Main |
|---|---|---|---|---|---|
| | | | | Main peak | peak |
| | Temp | | Time | retention | area |
| Sample | (° C.) | | (week) | (min) | (%) |
| h26D3 HC6 DS T91S T120K_T0 | −75° | C. | 0 | 10.906 | 99.87 |
| h26D3 HC6 DS T91S T120K_FT_3x | — | | 0 | 10.919 | 99.88 |
| h26D3 HC6 DS T91S T120K_+4 C._w 1 | +4° | C. | 1 | 10.913 | 99.87 |
| h26D3 HC6 DS T91S T120K_+4 C._w 2 | +4° | C. | 2 | 10.924 | 99.78 |
| h26D3 HC6 DS T91S T120K_+4 C._w 4 | +4° | C. | 4 | 10.941 | 99.74 |
| h26D3 HC6 DS T91S T120K_+25 C._w 1 | +25° | C. | 1 | 10.917 | 99.61 |
| h26D3 HC6 DS T91S T120K_+25 C._w 2 | +25° | C. | 2 | 10.923 | 99.65 |
| h26D3 HC6 DS T91S T120K_+25 C._w 4 | +25° | C. | 4 | 10.942 | 99.47 |
| h26D3 HC6 DS T91S T120K_+40 C._w 1 | +40° | C. | 1 | 10.914 | 99.75 |
| h26D3 HC6 DS T91S T120K_+40 C._w 2 | +40° | C. | 2 | 10.928 | 99.25 |
| h26D3 HC6 DS T91S T120K_+40 C._w 4 | +40° | C. | 4 | 10.950 | 94.01 |

TABLE 23
Analytical SEC from thermal stability study on h26D3 HC6 DS V11R T120K

| | | | | aSEC. | Main |
|---|---|---|---|---|---|
| | | | | Main peak | peak |
| | Temp | | Time | retention | area |
| Sample | (° C.) | | (week) | (min) | (%) |
| h26D3 HC6 DS V11R T120K_T0 | −75° | C. | 0 | 10.912 | 99.85 |
| h26D3 HC6 DS V11R T120K_FT_3x | — | | 0 | 10.919 | 99.85 |
| h26D3 HC6 DS V11R T120K_+4 C._w 1 | +4° | C. | 1 | 10.919 | 99.82 |
| h26D3 HC6 DS V11R T120K_+4 C._w 2 | +4° | C. | 2 | 10.930 | 99.78 |
| h26D3 HC6 DS V11R T120K_+4 C._w 4 | +4° | C. | 4 | 10.947 | 99.72 |
| h26D3 HC6 DS V11R T120K_+25 C._w 1 | +25° | C. | 1 | 10.923 | 99.53 |
| h26D3 HC6 DS V11R T120K_+25 C._w 2 | +25° | C. | 2 | 10.931 | 99.56 |
| h26D3 HC6 DS V11R T120K_+25 C._w 4 | +25° | C. | 4 | 10.948 | 99.51 |
| h26D3 HC6 DS V11R T120K_+40 C._w 1 | +40° | C. | 1 | 10.920 | 99.71 |
| h26D3 HC6 DS V11R T120K_+40 C._w 2 | +40° | C. | 2 | 10.934 | 98.77 |
| h26D3 HC6 DS V11R T120K_+40 C._w 4 | +40° | C. | 4 | 10.955 | 94.12 |

TABLE 24
Analytical SEC from thermal stability study on h26D3 HC6 DS V11R T91A

| | | | | aSEC. | Main |
|---|---|---|---|---|---|
| | | | | Main peak | peak |
| | Temp | | Time | retention | area |
| Sample | (° C.) | | (week) | (min) | (%) |
| h26D3 HC6 DS V11R T91A_T0 | −75° | C. | 0 | 10.910 | 99.91 |
| h26D3 HC6 DS V11R T91A_FT_3x | — | | 0 | 10.919 | 99.88 |
| h26D3 HC6 DS V11R T91A_+4 C._w 1 | +4° | C. | 1 | 10.918 | 99.86 |
| h26D3 HC6 DS V11R T91A_+4 C._w 2 | +4° | C. | 2 | 10.928 | 99.80 |
| h26D3 HC6 DS V11R T91A_+4 C._w 4 | +4° | C. | 4 | 10.944 | 99.82 |
| h26D3 HC6 DS V11R T91A_+25 C._w 1 | +25° | C. | 1 | 10.921 | 99.60 |
| h26D3 HC6 DS V11R T91A_+25 C._w 2 | +25° | C. | 2 | 10.929 | 99.69 |
| h26D3 HC6 DS V11R T91A_+25 C._w 4 | +25° | C. | 4 | 10.945 | 99.62 |
| h26D3 HC6 DS V11R T91A_+40 C._w 1 | +40° | C. | 1 | 10.918 | 99.77 |
| h26D3 HC6 DS V11R T91A_+40 C._w 2 | +40° | C. | 2 | 10.933 | 98.82 |
| h26D3 HC6 DS V11R T91A_+40 C._w 4 | +40° | C. | 4 | 10.952 | 94.03 |

As seen in FIG. 28, the main peak for all samples has a retention time at ˜11 min, as expected for a monomeric scFv. All samples have a high monomer content at all tested conditions, demonstrating that the stabilized DS design is maintained in all six variants.

### Example 18

### Immunogenicity in Silico of Additional Variants of hTfR1 Binding Molecules

The six scFv sequences analyzed in Example 17 (SEQ ID NO:305-310) and the variant “h26D3 HC6 DS, VL-first” (SEQ ID NO:118; Example 10) were subjected to an in silico analysis of immunogenicity risk, based on identification of potential T cell epitopes in the respective amino acid sequence. The assessment was done using iTope-Al (Abzena Ltd., Cambridge, UK). In addition, the sequences were analysed for homology to known T cell epitopes, previously identified by ex vivo EpiScreen™ analysis of other protein sequences. Homology scores against known T cell epitopes (Bryson et al (2010), BioDrugs 24 (1): 1-8) are shown as TCED in Table 25.

The immunogenicity assessment of the respective protein sequence was performed using overlapping 9mer peptides, tested against 46 MHC class Il allotypes used in the iTope-Al platform. Individual peptides, together spanning the whole sequence, were given a binding score from 0 to 3 for each allotype, and those scores were added together to provide a “Position Risk Score”. The “Total Score” for the respective test protein in Table 25 was calculated by adding the “Position Risk Scores” obtained for all individual peptides. The highest “Position Risk Score” for each sequence was denoted “Hotspot Max” as also shown in Table 25.

Taken together, all seven tested protein sequences show very similar results, indicating that no novel risk sites of concern were identified in any of the assessed variants of the TfR binding molecule.

TABLE 25
In silico immunogenicity assessment
of hTfR1-binding scFv molecules

| | | Total | Hotspot | TCED |
|---|---|---|---|---|
| scFv variant | SEQ ID NO: | Score | Max | Homology |
| h26D3 HC6 DS, VL-first | 118 | 102 | 19 | 6 |
| h26D3 HC6 DS V11R T91A | 310 | 134 | 24 | 7 |
| h26D3 HC6 DS V11R T120K | 309 | 105 | 19 | 6 |
| h26D3 HC6 DS T91S T120K | 308 | 117 | 19 | 7 |
| h26D3 HC6 DS T91S S122K | 307 | 116 | 19 | 7 |
| h26D3 HC6 DS T120K | 306 | 103 | 19 | 6 |
| h26D3 HC6 DS S122K | 305 | 102 | 19 | 6 |

### Example 19

### SPR Analysis of Binding of Additional scFv Variants to hTfR1 and cTfR1

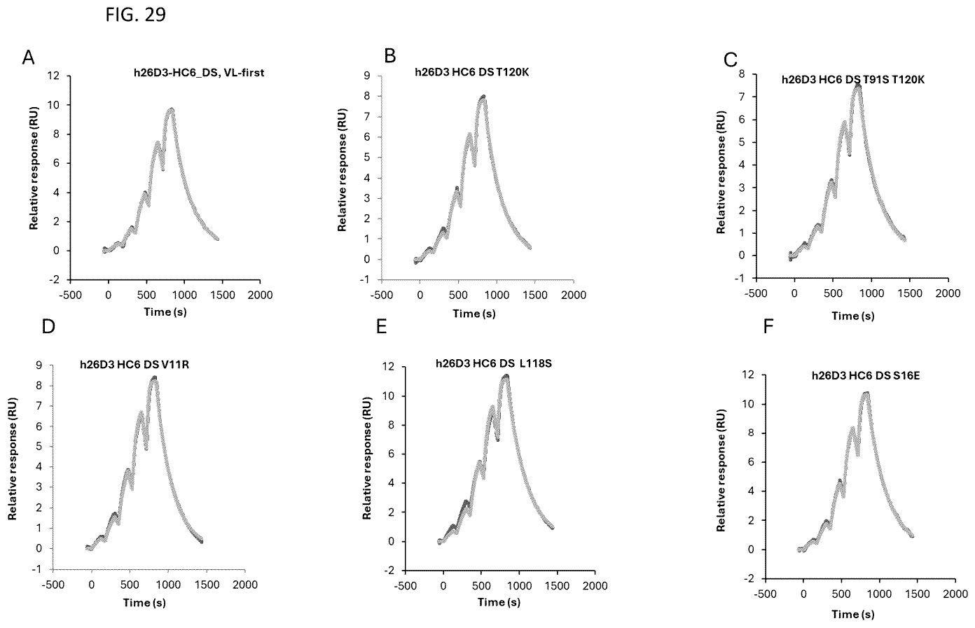

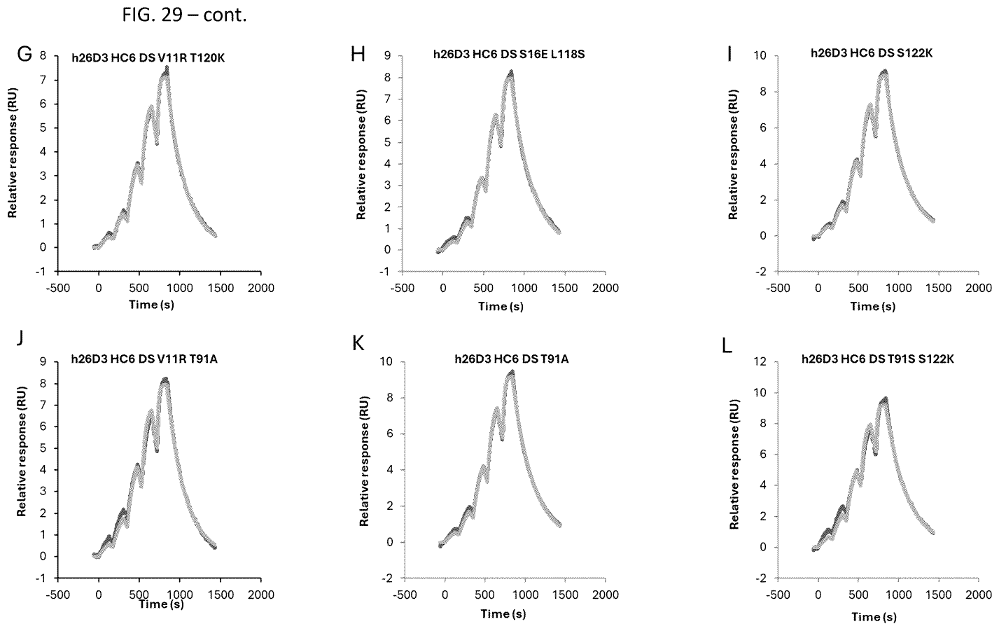

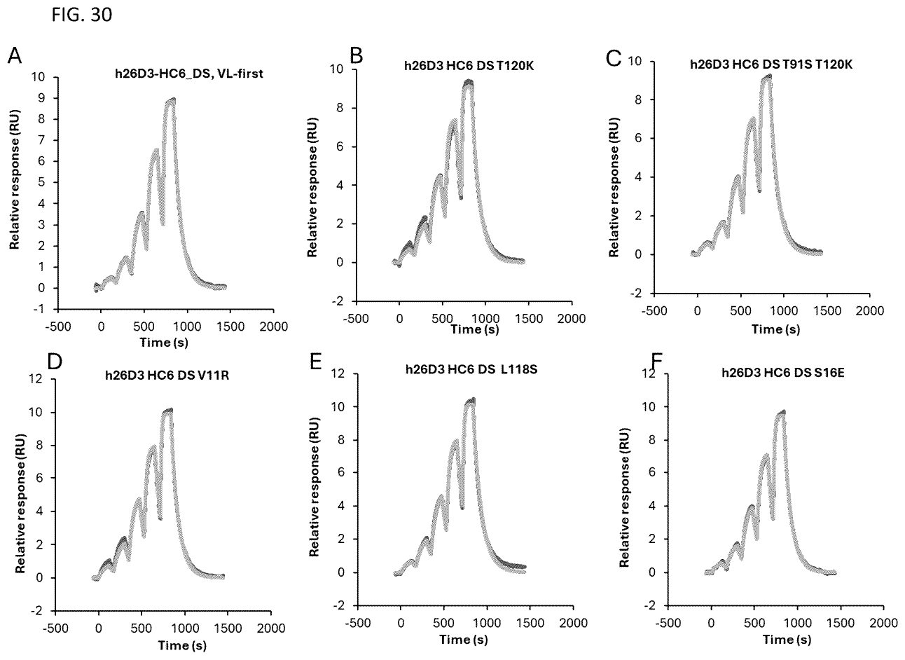

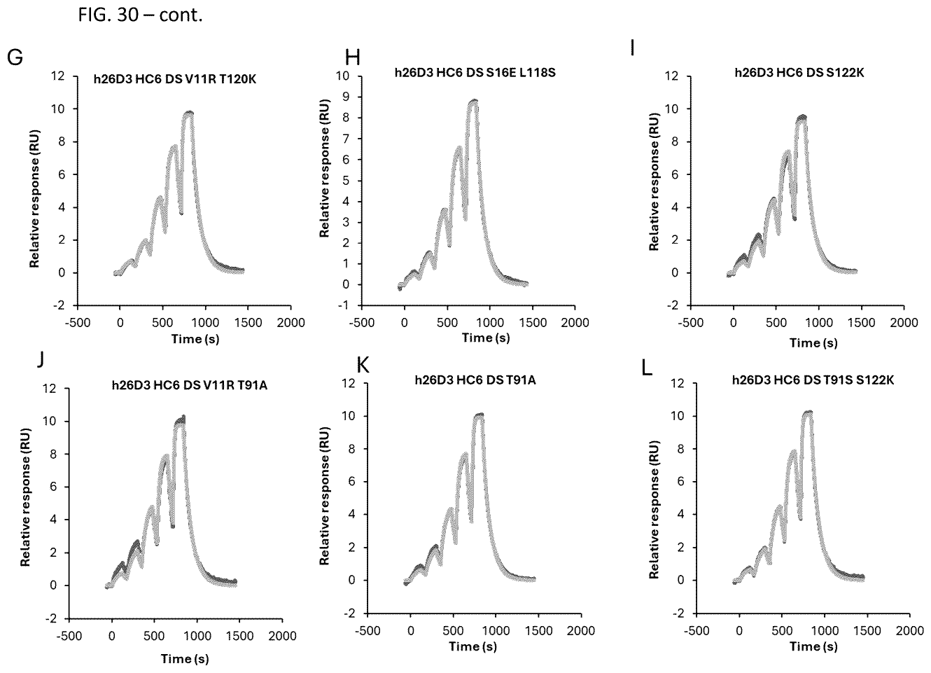

The 11 scFv molecules produced and purified as described in Example 16 and the variant “h26D3 HC6 DS, VL-first” (SEQ ID NO:118; Example 10) were evaluated for binding to human and cynomolgus TfR1 using SPR in a Biacore 8K instrument (Cytiva). For this, 30 μg/ml of DYKDDDDK Tag Monoclonal Antibody (FG4R) (ThermoFisher SCIENTIFIC. #14-6681-80) was immobilized on a CM5 sensor chip (Cytiva. #BR100399) using the amine coupling kit type 2 (Cytiva. #BR100633) according to the manufacturer's instruction. hTfR1 (SEQ ID NO:86) or cTfR1 (SEQ ID NO: 87), each containing a FLAG tag, was captured on the chip via injection of a 5 μg/ml solution for 60 s at 10 μl/min flow rate. Each scFv variant was injected over the chip using a 3-fold dilution series in five steps starting at 700 nM (hTfR1) or 1500 nM (cTfR1). The interaction was measured using the single cycle kinetics method with a contact time of 120 s at a flow rate of 30 μl/min followed by a dissociation time of 600 s. Regeneration of the surface between cycles was done by injecting 10 mM glycine-HCl, pH 1.7. The binding data was fitted to a 1:1 interaction model. The scFv variants were diluted in HBS-EP+ (Cytiva. #BR100669). Experiments were performed at 25° C. As expected, the binding to TfR was similar for all analyzed molecules. The results for binding to hTfR are shown in FIG. 29A-L, and to cTfR in FIG. 30A-L. The calculated K_{D} values for binding to hTfR and cTfR are given below in Tables 26 and 27, respectively.

TABLE 26
Affinity constants for binding to hTfR1

| scFv variant | ka (1/Ms) | kd (1/s) | KD (M) | N |
|---|---|---|---|---|
| h26D3-HC6_DS. VL-first | 6.35 ± 0.75 E+04 | 4.34 ± 0.14 E−03 | 6.89 ± 0.64 E−08 | 4 |
| h26D3 DS L118S | 6.62 ± 1.02 E+04 | 3.93 ± 0.04 E−03 | 6.00 ± 0.86 E−08 | 2 |
| h26D3 DS S16E | 5.77 ± 0.08 E+04 | 3.97 ± 0.01 E−03 | 6.89 ± 0.12 E−08 | 2 |
| h26D3 DS S16E L118S | 5.78 ± 0.05 E+04 | 3.87 ± 0.05 E−03 | 6.71 ± 0.14 E−08 | 2 |
| h26D3 DS S122K | 7.64 ± 1.03 E+04 | 4.07 ± 0.03 E−03 | 5.37 ± 0.69 E−08 | 2 |
| h26D3 DS T91A | 6.50 ± 0.04 E+04 | 3.88 ± 0.05 E−03 | 5.97 ± 0.11 E−08 | 2 |
| h26D3 DS T91S S122K | 7.62 ± 1.14 E+04 | 3.80 ± 0.05 E−03 | 5.05 ± 0.82 E−08 | 2 |
| h26D3 DS T91S T120K | 6.19 ± 0.12 E+04 | 4.09 ± 0.10 E−03 | 6.62 ± 0.30 E−08 | 2 |
| h26D3 DS T120K | 7.51 ± 2.02 E+04 | 4.29 ± 0.04 E−03 | 5.93 ± 1.65 E−08 | 2 |
| h26D3 DS V11R | 9.04 ± 2.74 E+04 | 4.65 ± 0.05 E−03 | 5.40 ± 1.69 E−08 | 2 |
| h26D3 DS V11R T91A | 7.61 ± 1.38 E+04 | 4.58 ± 0.13 E−03 | 6.13 ± 1.28 E−08 | 2 |
| h26D3 DS V11R T120K | 7.33 ± 0.29 E+04 | 4.41 ± 0.02 E−03 | 6.02 ± 0.26 E−08 | 2 |

TABLE 27
Affinity constants for binding to cTfR1

| scFv variant | ka (1/Ms) | kd (1/s) | KD (M) | N |
|---|---|---|---|---|
| h26D3-HC6_DS. VL-first | 4.76 ± 0.89 E+04 | 1.14 ± 0.02 E−02 | 2.46 ± 0.48 E−07 | 4 |
| h26D3 DS L118S | 4.40 ± 0.31 E+04 | 1.07 ± 0.07 E−02 | 2.43 ± 0.33 E−07 | 2 |
| h26D3 DS S16E | 4.38 ± 0.14 E+04 | 1.21 ± 0.07 E−02 | 2.77 ± 0.07 E−07 | 2 |
| h26D3 DS S16E L118S | 4.17 ± 0.09 E+04 | 1.09 ± 0.02 E−02 | 2.61 ± 0.01 E−07 | 2 |
| h26D3 DS S122K | 6.15 ± 0.78 E+04 | 1.03 ± 0.10 E−02 | 1.70 ± 0.38 E−07 | 2 |
| h26D3 DS T91A | 4.62 ± 0.29 E+04 | 1.11 ± 0.05 E−02 | 2.40 ± 0.04 E−07 | 2 |
| h26D3 DS T91S S122K | 4.86 ± 0.11 E+04 | 1.08 ± 0.02 E−02 | 2.21 ± 0.09 E−07 | 2 |
| h26D3 DS T91S T120K | 4.67 ± 0.02 E+04 | 1.15 ± 0.09 E−02 | 2.45 ± 0.18 E−07 | 2 |
| h26D3 DS T120K | 5.74 ± 0.06 E+04 | 1.13 ± 0.04 E−02 | 1.97 ± 0.09 E−07 | 2 |
| h26D3 DS V11R | 5.15 ± 0.69 E+04 | 1.13 ± 0.01 E−02 | 2.22 ± 0.27 E−07 | 2 |
| h26D3 DS V11R T91A | 5.81 ± 0.01 E+04 | 1.20 ± 0.14 E−02 | 2.06 ± 0.24 E−07 | 2 |
| h26D3 DS V11R T120K | 6.13 ± 0.89 E+04 | 1.24 ± 0.21 E−02 | 2.01 ± 0.05 E−07 | 2 |

Next, in order to compare binding parameters with more certainty, a follow-up SPR analysis with additional replicates (N=4), was conducted for six selected molecules (SEQ ID NO:305-310) and the variant “h26D3 HC6 DS, VL-first”. The selected molecules were produced as described above. As shown in Table 28 below, the measured affinity K_{D} values are very similar between all selected variants.

TABLE 28
Affinity constants for binding to hTfR1 measured in SPR with 4 replicates

| scFv variant | ka (1/Ms) | kd (1/s) | KD (M) | N |
|---|---|---|---|---|
| h26D3-HC6_DS, VL-first | 6.71 ± 0.46 E+04 | 4.39 ± 1.73 E−03 | 6.62 ± 2.77 E−08 | 4 |
| h26D3 DS S122K | 8.58 ± 3.29 E+04 | 4.82 ± 0.71 E−03 | 6.26 ± 2.41 E−08 | 4 |
| h26D3 DS T91S S122K | 6.70 ± 0.37 E+04 | 4.63 ± 0.37 E−03 | 6.95 ± 0.88 E−08 | 4 |
| h26D3 DS T91S T120K | 8.72 ± 1.88 E+04 | 4.65 ± 0.36 E−03 | 5.55 ± 1.41 E−08 | 4 |
| h26D3 DS T120K | 6.73 ± 0.47 E+04 | 4.78 ± 0.27 E−03 | 7.13 ± 0.75 E−08 | 4 |
| h26D3 DS V11R T91A | 7.49 ± 1.60 E+04 | 4.19 ± 0.64 E−03 | 5.87 ± 1.78 E−08 | 4 |
| h26D3 DS V11R T120K | 9.57 ± 2.00 E+04 | 4.49 ± 0.79 E−03 | 4.86 ± 1.34 E−08 | 4 |

### Example 20

### PE-ADA Assay in Serum

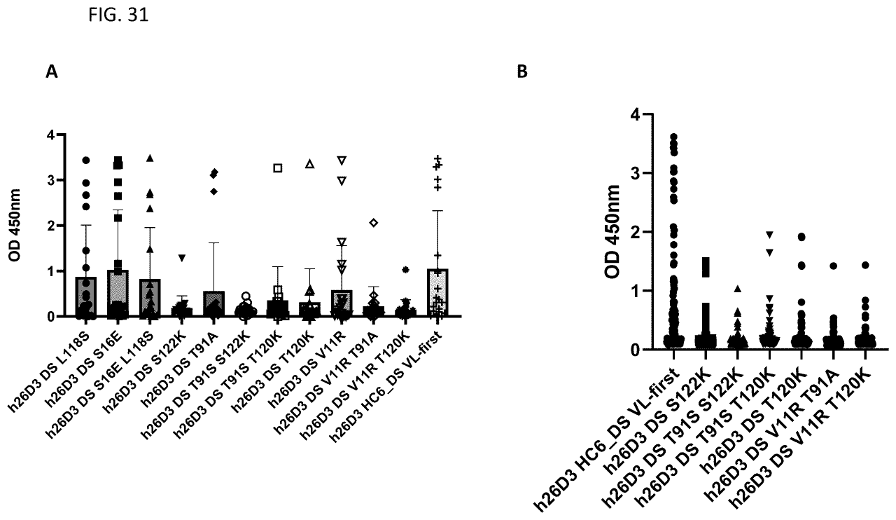

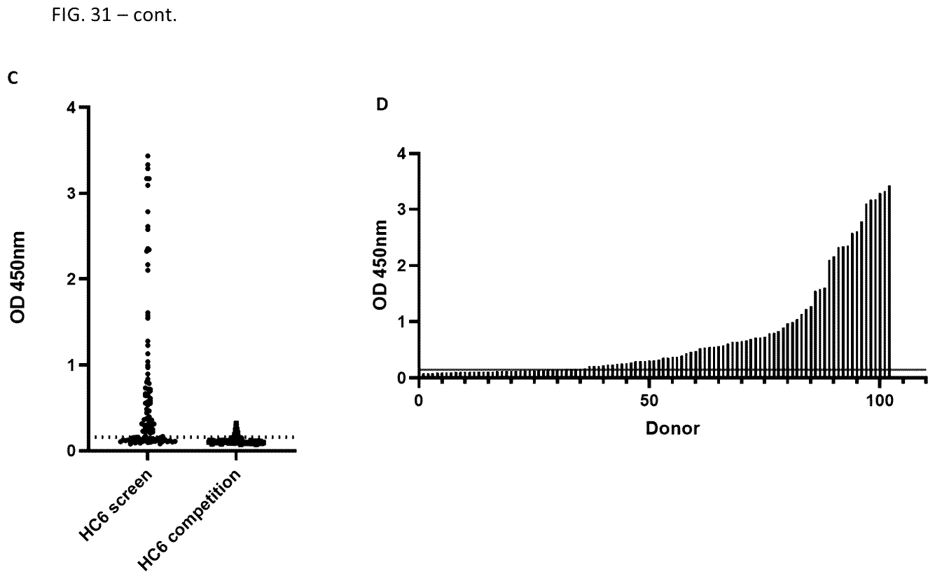

To investigate the degree of reactivity from pre-existing anti-drug antibodies among non-exposed individuals to a set of scFv molecules, a representative subset of 21 human serum samples was used to screen for binding response to 11 new scFv variants (SEQ ID NO:305-315) produced as described in Example 16. The response was measured using a direct ELISA as described below. Results from the direct ELISA screen show that the reactivity frequences differ between the screened variants, and that the response frequency is reduced for some of the investigated variants in line with previous findings (Johansson et al (2023), mAbs 15 (1): 2215887). The binding reactivity is shown in FIG. 31A.

From the results of this initial ELISA screen, six variants (SEQ ID NO: 305-310) were selected and subjected to additional ELISA measurements of PE-ADA reactivity in a larger set of 107 human serum samples, as described below. As seen in FIG. 31B, all six variants have a reduced reactivity compared to “h26D3-HC6_DS, VL-first” (SEQ ID NO: 118).

To further evaluate the observed PE-ADA reactivity, a competition ELISA assay was conducted, as described below. The result is shown in FIG. 31C-D and verify that the reactivity is directed to “h26D3-HC6_DS, VL-first” and could be largely blocked by competition with “h26D3-HC6_DS, VL-first” in solution.

Serum samples: Serum was obtained from healthy donors giving blood during 2023-2025 (ethical permission D-nr 2018/804-31 from the regional ethics board in Stockholm, Sweden).

Screening of PE-ADA response by direct ELISA: 96-well half-area ELISA plates (Corning. #3690) were coated with 50 μl per well of 3.4 nM scFv variant in PBS for 2 h at RT with shaking at 600-900 rpm. Two wells per plate were instead coated with anti-human IgG F (ab′) 2 (JacksonImmuno; #109-005-097) to serve as positive control wells. For blocking, plates were emptied and tapped dry on paper towels prior to addition of 150 μl per well of blocking solution (1% BSA, 0.01% Tween20 in PBS). Plates were incubated with blocking solution for at least 1 h at RT. shaking at 600-900 rpm. Plates were then washed 4 times with 150 μl per well of ELISA wash buffer with an automated plate washer (Tecan). Plates were tapped dry on paper towels and 25 μl of LowCross buffer (Candor Bioscience; #100-050) were added to each well. Then, 25 μl of 1:20 diluted serum samples in LowCross Buffer were added in duplicates to reach a final serum dilution of 1:40. Rituximab diluted in LowCross buffer was added to positive control wells. Samples were incubated for 25 min at RT without shaking. Plates were washed 4 times as above prior to addition of 50 μl per well of HRP-F (ab′) 2 goat anti-human IgG Fcγ specific polyclonal antibody (JacksonImmuno #109-036-008) diluted 1:5000 in LowCross buffer for 1 h at RT without shaking. Plates were washed 4 times as above prior to addition of 50 μl per well of TMB (Neogen; #331177). The colorimetric signal was allowed to develop for 5 min prior to stopping with 50 μl per well of sulfuric acid (Honeywell; #35354-1L). Plates were read immediately after addition of sulfuric acid on a plate reader at 450 nM (Tecan).

Confirmatory competition ELISA assay: The competition ELISA was performed analogous to the direct ELISA screening assay described above, with the following adaptation: during blocking of plates, serum diluted 1:20 in LowCross buffer was either mixed with an equal volume of LowCross buffer or with an equal volume of 3400 nM “h26D3-HC6_DS, VL-first” scFv, resulting in a final serum dilution of 1:40 and 500× excess scFv compared to coating. The samples were pre-incubated in 96-well PP plate for 1 h, with shaking at 900 rpm. After blocking and washing, duplicates of either serum only or serum with soluble scFv were added to the plate and incubated for 25 min at RT without shaking. From here, the protocol for screening was followed.

Calculation of assay cut-off points: In order to determine the assay cut-off point, technical outliers with a % CV larger than 20 were first removed. For this, the mean of the sample duplicates was calculated, followed by calculation of the standard deviation. % CV was calculated as % CV=STDEV/MEAN*100.

The sample population minus technical outliers was then analyzed for statistical outliers by ROUT analysis in GraphPad Prism (Q=1%; allowing for 1% of false positive identified outliers). The sample population minus technical and statistical outliers made up the pseudo-negative population and was used to calculate the assay cut-off point. In case of high PE-ADA prevalence, the cut-off point was based on the competition assay: cut-off point=mean (of pseudo negative population)+2.33×STDEV (of pseudo negative population), allowing for a 1% false positive rate.

In case of low PE-ADA prevalence, the cut-off point can be directly calculated from the screening assay: cut-off point=mean (screening population with outliers removed)+1.645× STDEV (of screening population with outliers removed), allowing for a 5% false positive rate.

### Itemized Listing of Embodiments

1. A transferrin receptor 1 (TfR1) binding molecule, which
is capable of selective binding to an epitope located in the protease-like domain of TfR1 defined by amino acid residues 121-183 and 384-605 in SEQ ID NO: 85,
comprises an immunoglobulin heavy chain variable region (VH) and an immunoglobulin light chain variable region (VL), said VH and VL regions forming a VH/VL pair comprising an antigen-binding surface, in which said antigen-binding surface provides the binding molecule with the capacity to bind selectively to said epitope; and
comprises one first cysteine residue in said VH region and one second cysteine residue in said VL region, said first and second cysteine residues being arranged such that they form a disulfide bridge connecting the VH and VL regions.

2. Binding molecule according to item 1, wherein said first cysteine residue is located at an amino acid position selected from VH position 39-49, such as selected from VH position 41-47, such as selected from VH position 43-45, such as at VH position 44, all as determined by reference to the Kabat numbering scheme.

3. Binding molecule according to item 1 or 2, wherein said second cysteine residue is located at an amino acid position selected from VL position 95-105, such as selected from VL position 97-103, such as selected from VL position 99-101, such as at VL position 100, all as determined by reference to the Kabat numbering scheme.

4. Binding molecule according to item 2 or 3, wherein said first cysteine residue is located at VH position 44 and said second cysteine residue is located at VL position 100, as determined by reference to the Kabat numbering scheme.

5. Binding molecule according to any preceding item, said epitope comprising or consisting of the amino acid residues 150, 151, 154, 158, 159, 161, 163 and 385 in SEQ ID NO:85.

6. Binding molecule according to any preceding item, in which said antigen-binding surface is composed of three complementarity-determining regions (CDRs) from said VH region and three CDRs from said VL region, and in which said CDRs comprise the following:

VHCDR1:
(SEQ ID NO: 1)
X1X2NMX3,

wherein
X1 is selected from D and A;
X2 is selected from Y and A; and
X3 is selected from D and A;

VHCDR2:
(SEQ ID NO: 2)
X4INPX5X6X7TTSX8X9X10KFKG,

wherein
X4 is selected from D and A;
X5 is selected from D, N and A;
X6 is selected from Y and A;
X7 is selected from D and A;
X8 is selected from Y and A;
X9 is selected from N and S; and
X10 is selected from E and Q;

VLCDR1:
(SEQ ID NO: 4)
KSSQSLLX11SX12NX13KNX14LA,

wherein
X11 is selected from Y and A;
X12 is selected from T and S;
X13 is selected from Q and R; and
X14 is selected from Y and A;

VLCDR2:
(SEQ ID NO: 5)
X15ASTRES

wherein
X15 is selected from W and A; and

VLCDR3:
(SEQ ID NO: 6)
QQX16X17X18X19PX20T

wherein
X16 is selected from Y and A;
X17 is selected from F and Y;
X18 is selected from I and N;
X19 is selected from Y and A; and
X20 is selected from R and Y.

7. Binding molecule according to item 6, said CDRs further comprising

VHCDR3:
(SEQ ID NO: 3)
GGX21SGSSX22X23HPMX24X25

wherein
X21 is selected from Y and A;
X22 is selected from Y and A;
X23 is selected from Y and A;
X24 is selected from D and A; and
X25 is selected from Y and A.

8. Binding molecule according to any one of items 6-7, in which said VHCDR2 is:

VHCDR2:
(SEQ ID NO: 7)
X4INPX5X6X7TTSX8NEKFKG,

wherein
X4 is selected from D and A;
X5 is selected from D and A;
X6 is selected from Y and A;
X7 is selected from D and A; and
X8 is selected from Y and A.

9. Binding molecule according to any one of items 6-8, in which said VLCDR1 is:

VLCDR1:
(SEQ ID NO: 8)
KSSQSLLX11STNQKNX14LA,

wherein
X11 is selected from Y and A; and
X14 is selected from Y and A.

10. Binding molecule according to any one of items 6-9, in which said VLCDR3 is:

VLCDR3:
(SEQ ID NO: 9)
QQX16FIX19PRT

wherein X16 is selected from Y and A; and
X19 is selected from Y and A.

11. Binding molecule according to any one of items 6-10, in which the amino acid sequence of said VHCDR1 is selected from the group consisting of SEQ ID NO:10 and 16-18.

12. Binding molecule according to any one of items 6-11, in which the amino acid sequence of said VHCDR2 is selected from the group consisting of SEQ ID NO:11, 19-23 and 34, for example selected from the group consisting of SEQ ID NO:11 and 19-23.

13. Binding molecule according to any one of items 6-12, in which the amino acid sequence of said VHCDR3 is selected from the group consisting of SEQ ID NO:12, 24-28 and 35, for example selected from the group consisting of SEQ ID NO:12 and 24-28.

14. Binding molecule according to any one of items 6-13, in which the amino acid sequence of said VLCDR1 is selected from the group consisting of SEQ ID NO:13, 29, and 36, for example selected from the group consisting of SEQ ID NO:13, 29 and 30.

15. Binding molecule according to any one of items 6-14, in which the amino acid sequence of said VLCDR2 is selected from the group consisting of SEQ ID NO:14 and 31.

16. Binding molecule according to any one of items 6-15, in which the amino acid sequence of said VLCDR3 is selected from the group consisting of SEQ ID NO:15, 32, 33 and 37, for example selected from the group consisting of SEQ ID NO:15, 32 and 33.

17. Binding molecule according to any one of items 6-16, in which the amino acid sequences of the six CDRs are the following:

VHCDR1:
(SEQ ID NO: 10)
DYNMD,
VHCDR2:
(SEQ ID NO: 11)
DINPDYDTTSYNEKFKG,
VHCDR3:
(SEQ ID NO: 12)
GGYSGSSYYHPMDY,
VLCDR1:
(SEQ ID NO: 13)
KSSQSLLYSTNQKNYLA,
VLCDR2:
(SEQ ID NO: 14)
WASTRES,
and
VLCDR3:
(SEQ ID NO: 15)
QQYFIYPRT.

18. Binding molecule according to any one of items 6-16, in which the amino acid sequences of the six CDRs are the following:

VHCDR1:
(SEQ ID NO: 10)
DYNMD,
VHCDR2:
(SEQ ID NO: 21)
DINPDADTTSYNEKFKG,
VHCDR3:
(SEQ ID NO: 12)
GGYSGSSYYHPMDY,
VLCDR1:
(SEQ ID NO: 13)
KSSQSLLYSTNQKNYLA,
VLCDR2:
(SEQ ID NO: 14)
WASTRES,
and
VLCDR3:
(SEQ ID NO: 15)
QQYFIYPRT.

19. Binding molecule according to item 6, in which the amino acid sequences of the six CDRs are the following:

VHCDR1:
(SEQ ID NO: 10)
DYNMD,
VHCDR2:
(SEQ ID NO: 34)
DINPNYDTTSYSQKFKG,
VHCDR3:
(SEQ ID NO: 35)
SEAGNYYWYFDV,
VLCDR1:
(SEQ ID NO: 36)
KSSQSLLYSSNRKNYLA,
VLCDR2:
(SEQ ID NO: 14)
WASTRES,
and
VLCDR3:
(SEQ ID NO: 37)
QQYYNYPYT.

20. Binding molecule according to any preceding item, wherein said VH region comprises or consists of an amino acid sequence selected from
(i) the group consisting of SEQ ID NO:88-103, for example the group consisting of SEQ ID NO: 88-101, for example the group consisting of SEQ ID NO:88 and 94; and
(ii) a sequence having at least 80%, at least 90%, at least 92%, at least 94%, at least 95%, at least 96%, at least 97%, at least 98% or at least 99% identity to a sequence defined in (i), provided that the sequences of the CDR regions are 100% identical to those of the CDR regions in a sequence defined in (i), and provided that the sequence comprises a cysteine residue at position 44.

21. Binding molecule according to any preceding item, wherein said VL region comprises or consists of an amino acid sequence selected from
(i) the group consisting of SEQ ID NO:105-113, for example the group consisting of SEQ ID NO:105-111; and
(ii) a sequence having at least 80%, at least 90%, at least 92%, at least 94%, at least 95%, at least 96%, at least 97%, at least 98% or at least 99% identity to a sequence defined in (i), provided that the sequences of the CDR regions are 100% identical to those of the CDR regions in a sequence defined in (i), and provided that the sequence comprises a cysteine residue at position 106.

22. Binding molecule according to any one of items 20-21, wherein said VH region is as defined in item 20 and said VL region is as defined in item 21.

23. Binding molecule according to item 22, in which said VH region comprises SEQ ID NO: 88 and said VL region comprises a sequence selected from SEQ ID NO:105-111.

24. Binding molecule according to item 22, in which said VH region comprises a sequence selected from SEQ ID NO:88-101 and said VL region comprises SEQ ID NO: 105.

25. Binding molecule according to any one of items 23-24, in which said VH region comprises SEQ ID NO:88 and said VL region comprises SEQ ID NO:105.

26. Binding molecule according to any one of items 23-24, in which said VH region comprises SEQ ID NO:94 and said VL region comprises SEQ ID NO:105.

27. Binding molecule according to any one of items 1-5, in which said antigen-binding surface is composed of three complementarity-determining regions (CDRs) from said VH region and three CDRs from said VL region, and in which said CDRs comprise the following:

VHCDR1:
(SEQ ID NO: 38)
NYWLG,
VHCDR2:
(SEQ ID NO: 39)
DIFPGSDNTYYNEKFKG,
VHCDR3:
(SEQ ID NO: 40)
SGNFYAMDY,
VLCDR1:
(SEQ ID NO: 41)
SASSSVNYMN,
VLCDR2:
(SEQ ID NO: 42)
DTSKLAS,
and
VLCDR3:
(SEQ ID NO: 43)
FQGSGYPFT.

28. Binding molecule according to item 27, wherein said VH region comprises or consists of an amino acid sequence selected from SEQ ID NO:104 and a sequence having at least 80%, at least 90%, at least 92%, at least 94%, at least 95%, at least 96%, at least 97%, at least 98% or at least 99% identity to SEQ ID NO:104, provided that the sequences of the CDR regions are 100% identical to those of the CDR regions in SEQ ID NO:104, and provided that the sequence comprises a cysteine residue at position 44.

29. Binding molecule according to any one of items 27-28, wherein said VL region comprises or consists of an amino acid sequence selected from SEQ ID NO:114 and a sequence having at least 80%, at least 90%, at least 92%, at least 94%, at least 95%, at least 96%, at least 97%, at least 98% or at least 99% identity to SEQ ID NO:114, provided that the sequences of the CDR regions are 100% identical to those of the CDR regions in SEQ ID NO:114, and provided that the sequence comprises a cysteine residue at position 99.

30. Binding molecule according to any one of items 28-29, wherein said VH region is as defined in item 28 and said VL region is as defined in item 29.

31. Binding molecule according to any preceding item, in which said VH/VL pair forms part of an antibody construct.

32. Binding molecule according to item 31, in which said antibody construct has more than one binding specificity.

33. Binding molecule according to item 32, in which said antibody construct is bispecific.

34. Binding molecule according to item 33, wherein the VH/VL pair as defined in any preceding item is present in an antibody fragment selected from the group consisting of a Fab fragment, a single chain Fab (scFab) fragment, an Fv fragment and a single chain (scFv) fragment.

35. Binding molecule according to item 34, wherein said antibody fragment is an scFv.

36. Binding molecule according to any one of items 32-35, further comprising an antibody or antigen binding fragment thereof capable of selective binding to a target present in the brain of a mammal.

37. Binding molecule according to item 36, wherein said target is selected from the group consisting of amyloid-β peptide or derivatives or fragments thereof, alpha-synuclein or derivatives or fragments thereof, TAR DNA-binding protein 43 (TDP-43) or derivatives or fragments thereof, triggering receptor expressed on myeloid cells 2 (TREM2), beta-secretase 1 (BACE1), superoxide dismutase (SOD), huntingtin, transthyretin, P-secretase 1, epidermal growth factor, epidermal growth factor receptor 2, Tau, phosphorylated Tau or fragments thereof, apolipoprotein E4, CD20, prion protein, leucine rich repeat kinase 2, parkin, presenilin 2, gamma secretase, death receptor 6, amyloid-β precursor protein, p75 neurotrophin receptor, neuregulin and caspase 6.

38. Binding molecule according to item 37, wherein said target is selected from the group consisting of amyloid-β peptide or derivatives or fragments thereof, alpha-synuclein or derivatives or fragments thereof, TAR DNA-binding protein 43 (TDP-43) or derivatives or fragments thereof, triggering receptor expressed on myeloid cells 2 (TREM2), Tau, phosphorylated Tau or fragments thereof and apolipoprotein E4.

39. Binding molecule according to item 38, wherein said target is selected from the group consisting of amyloid-β peptide or derivatives or fragments thereof, alpha-synuclein or derivatives or fragments thereof and TAR DNA-binding protein 43 (TDP-43) or derivatives or fragments thereof.

40. Binding molecule according to any one of items 36-39, wherein said antibody or antigen binding fragment thereof capable of selective binding to a target present in the brain of a mammal is an anti-AB antibody, for example an antibody selected from the group consisting of lecanemab, gantenerumab, aducanumab, donanemab, PBD-C06 and KHK6640.

41. Binding molecule according to any one of items 36-39, wherein said antibody or antigen binding fragment thereof capable of selective binding to a target present in the brain of a mammal is an anti-alpha-synuclein antibody, for example an antibody selected from the group consisting of prasinezumab, UCB7853, Lu AF82422, TAK-341 and BAN0805.

42. Pharmaceutical composition, comprising a binding molecule according to any preceding item and a pharmaceutically acceptable carrier or excipient.

43. A binding molecule according to any one of items 1-41 or a composition according to item 42 for use in treatment, such as for use in therapeutic treatment or for use in prophylactic treatment.

44. A binding molecule according to any one of items 1-41 or a composition according to item 42 for use in diagnosis in vivo or prognosis in vivo.

45. A binding molecule or composition for use according to any one of items 43-44, wherein said therapy, prophylaxis, in vivo diagnosis or in vivo prognosis is with respect to a neurodegenerative disorder, for example a disorder selected from Alzheimer's disease and other disorders associated with Aβ protein aggregation, traumatic brain injury (TBI), Lewy body dementia (LBD), Down's syndrome (DS), amyotrophic lateral sclerosis (ALS), frontotemporal dementia, tauopathy, systemic amyloidosis, atherosclerosis, Parkinson's disease (PD), Parkinson's disease dementia (PDD), the Lewy body variant of Alzheimer's disease, multiple system atrophy, psychosis, schizophrenia, Creutzfeldt-Jakob disease, Huntington's disease, and familial amyloid neuropathy.

46. A binding molecule or composition for use according to item 45, wherein said therapy, prophylaxis, in vivo diagnosis or in vivo prognosis is with respect to a disorder selected from Alzheimer's disease and other disorders associated with AB protein aggregation, Lewy body dementia (LBD), Down's syndrome (DS), amyotrophic lateral sclerosis (ALS), frontotemporal dementia, tauopathy, Parkinson's disease (PD), Parkinson's disease dementia (PDD) and the Lewy body variant of Alzheimer's disease.

47. A binding molecule or composition for use according to item 46, wherein said therapy, prophylaxis, in vivo diagnosis or in vivo prognosis is with respect to a disorder selected from Alzheimer's disease and other disorders associated with AB protein aggregation, Lewy body dementia (LBD), amyotrophic lateral sclerosis (ALS) and Parkinson's disease (PD).

48. A binding molecule or composition for use according to item 47, wherein said therapy, prophylaxis, in vivo diagnosis or in vivo prognosis is with respect to Alzheimer's disease.

49. A binding molecule or composition for use according to any one of items 43-44, wherein said therapy, prophylaxis, in vivo diagnosis or in vivo prognosis is with respect to a disorder selected from brain cancer, multiple sclerosis and lysosomal storage diseases.

50. A method of therapeutic or prophylactic treatment of a mammal having, or being at risk of developing, a disorder, said method comprising administering to said mammal a therapeutically effective amount of a binding molecule according to any one of items 1-41 or a composition according to item 42.

51. A method according to item 50, wherein said disorder is a neurodegenerative disorder, for example a neurodegenerative disorder as defined in any one of items 43-46.

52. A method according to item 50, wherein said disorder is as defined in item 47.

1. A transferrin receptor 1 (TfR1) binding molecule, which
is capable of selective binding to an epitope located in the protease-like domain of TfR1 defined by amino acid residues 121-183 and 384-605 in SEQ ID NO: 85,
comprises an immunoglobulin heavy chain variable region (VH) and an immunoglobulin light chain variable region (VL), said VH and VL regions forming a VH/VL pair comprising an antigen-binding surface, in which said antigen-binding surface provides the binding molecule with the capacity to bind selectively to said epitope; and
comprises one first cysteine residue in said VH region and one second cysteine residue in said VL region, said first and second cysteine residues being arranged such that they form a disulfide bridge connecting the VH and VL regions.

2. Binding molecule according to claim 1, wherein said first cysteine residue is located at an amino acid position selected from VH position 39-49, such as selected from VH position 41-47, such as selected from VH position 43-45, such as at VH position 44, all as determined by reference to the Kabat numbering scheme.

3. Binding molecule according to claim 1 or 2, wherein said second cysteine residue is located at an amino acid position selected from VL position 95-105, such as selected from VL position 97-103, such as selected from VL position 99-101, such as at VL position 100, all as determined by reference to the Kabat numbering scheme.

4. Binding molecule according to any preceding claim, in which said antigen-binding surface is composed of three complementarity-determining regions (CDRs) from said VH region and three CDRs from said VL region, and in which said CDRs comprise the following:
VHCDR1:
(SEQ ID NO: 1)
X1X2NMX3,
wherein
X1 is selected from D and A;
X2 is selected from Y and A; and
X3 is selected from D and A;
VHCDR2:
(SEQ ID NO: 2)
X4INPX5X6X7TTSX8X9X10KFKG,
wherein
X4 is selected from D and A;
X5 is selected from D, N and A;
X6 is selected from Y and A;
X7 is selected from D and A;
X8 is selected from Y and A;
X9 is selected from N and S; and
X10 is selected from E and Q;
VLCDR1:
(SEQ ID NO: 4)
KSSQSLLX11SX12NX13KNX14LA,
wherein
X11 is selected from Y and A;
X12 is selected from T and S;
X13 is selected from Q and R; and
X14 is selected from Y and A;
VLCDR2:
(SEQ ID NO: 5)
X15ASTRES
wherein
X15 is selected from W and A; and
VLCDR3:
(SEQ ID NO: 6)
QQX16X17X18X19PX20T
wherein
X16 is selected from Y and A;
X17 is selected from F and Y;
X18 is selected from I and N;
X19 is selected from Y and A; and
X20 is selected from R and Y;
optionally further comprising
VHCDR3:
(SEQ ID NO: 3)
GGX21SGSSX22X23HPMX24X25
wherein
X21 is selected from Y and A;
X22 is selected from Y and A;
X23 is selected from Y and A;
X24 is selected from D and A; and
X25 is selected from Y and A.

5. Binding molecule according to claim 4, in which the amino acid sequences of the six CDRs are the following:
VHCDR1:
(SEQ ID NO: 10)
DYNMD,
VHCDR2:
(SEQ ID NO: 11)
DINPDYDTTSYNEKFKG,
VHCDR3:
(SEQ ID NO: 12)
GGYSGSSYYHPMDY,
VLCDR1:
(SEQ ID NO: 13)
KSSQSLLYSTNQKNYLA,
VLCDR2:
(SEQ ID NO: 14)
WASTRES,
and
VLCDR3:
(SEQ ID NO: 15)
QQYFIYPRT.

6. Binding molecule according to claim 4, in which the amino acid sequences of the six CDRs are the following:
VHCDR1:
(SEQ ID NO: 10)
DYNMD,
VHCDR2:
(SEQ ID NO: 21)
DINPDADTTSYNEKFKG,
VHCDR3:
(SEQ ID NO: 12)
GGYSGSSYYHPMDY,
VLCDR1:
(SEQ ID NO: 13)
KSSQSLLYSTNQKNYLA,
VLCDR2:
(SEQ ID NO: 14)
WASTRES,
and
VLCDR3:
(SEQ ID NO: 15)
QQYFIYPRT.

7. Binding molecule according to any preceding claim, wherein said VH region comprises or consists of an amino acid sequence selected from
(i) the group consisting of SEQ ID NO:88-103, for example the group consisting of SEQ ID NO:88-101, for example the group consisting of SEQ ID NO:88 and 94; and
(ii) a sequence having at least 80%, at least 90%, at least 92%, at least 94%, at least 95%, at least 96%, at least 97%, at least 98% or at least 99% identity to a sequence defined in (i), provided that the sequences of the CDR regions are 100% identical to those of the CDR regions in a sequence defined in (i), and provided that the sequence comprises a cysteine residue at position 44.

8. Binding molecule according to any preceding claim, wherein said VL region comprises or consists of an amino acid sequence selected from
(i) the group consisting of SEQ ID NO:105-113, for example the group consisting of SEQ ID NO:105-111; and
(ii) a sequence having at least 80%, at least 90%, at least 92%, at least 94%, at least 95%, at least 96%, at least 97%, at least 98% or at least 99% identity to a sequence defined in (i), provided that the sequences of the CDR regions are 100% identical to those of the CDR regions in a sequence defined in (i), and provided that the sequence comprises a cysteine residue at position 106.

9. Binding molecule according to any one of claims 7-8, wherein said VH region is as defined in claim 7 and said VL region is as defined in claim 8.

10. Binding molecule according to any one of claims 8-9, in which said VH region comprises SEQ ID NO:94 and said VL region comprises SEQ ID NO:105.

11. Binding molecule according to any preceding claim, in which said VH/VL pair forms part of an antibody construct, for example a bispecific antibody construct.

12. Binding molecule according to claim 11, wherein the VH/VL pair as defined in any preceding claim is present in an antibody fragment selected from the group consisting of a Fab fragment, a single chain Fab (scFab) fragment, an Fv fragment and a single chain (scFv) fragment, for example an scFv.

13. Binding molecule according to any one of claims 11-12, further comprising an antibody or antigen binding fragment thereof capable of selective binding to a target present in the brain of a mammal, for example wherein said target is selected from the group consisting of amyloid-β peptide or derivatives or fragments thereof, alpha-synuclein or derivatives or fragments thereof, TAR DNA-binding protein 43 (TDP-43) or derivatives or fragments thereof, triggering receptor expressed on myeloid cells 2 (TREM2), beta-secretase 1 (BACE1), superoxide dismutase (SOD), huntingtin, transthyretin, P-secretase 1, epidermal growth factor, epidermal growth factor receptor 2, Tau, phosphorylated Tau or fragments thereof, apolipoprotein E4, CD20, prion protein, leucine rich repeat kinase 2, parkin, presenilin 2, gamma secretase, death receptor 6, amyloid-β precursor protein, p75 neurotrophin receptor, neuregulin and caspase 6.

14. Pharmaceutical composition, comprising a binding molecule according to any preceding claim and a pharmaceutically acceptable carrier or excipient.

15. A binding molecule according to any one of claims 1-13 or a composition according to claim 14 for use in treatment, such as for use in therapeutic treatment or for use in prophylactic treatment, or for use in diagnosis in vivo or prognosis in vivo, for example wherein said therapy, prophylaxis, in vivo diagnosis or in vivo prognosis is with respect to a neurodegenerative disorder, for example a disorder selected from Alzheimer's disease and other disorders associated with Aβ protein aggregation, traumatic brain injury (TBI), Lewy body dementia (LBD), Down's syndrome (DS), amyotrophic lateral sclerosis (ALS), frontotemporal dementia, tauopathy, systemic amyloidosis, atherosclerosis, Parkinson's disease (PD), Parkinson's disease dementia (PDD), the Lewy body variant of Alzheimer's disease, multiple system atrophy, psychosis, schizophrenia, Creutzfeldt-Jakob disease, Huntington's disease, and familial amyloid neuropathy.


SEQUENCE LISTING (315 sequences)

SEQ ID NO: 1 - skipped

SEQ ID NO: 2 - protein, 17 aa, synthetic construct
  VARIANT 1: note = D or A
  VARIANT 5: note = D or N or A
  VARIANT 6: note = Y or A
  VARIANT 7: note = D or A
  VARIANT 11: note = Y or A
  VARIANT 12: note = N or S
  VARIANT 13: note = E or Q
XINPXXXTTSXXXKFKG

SEQ ID NO: 3 - protein, 14 aa, synthetic construct
  VARIANT 3: note = Y or A
  VARIANT 8: note = Y or A
  VARIANT 9: note = Y or A
  VARIANT 13: note = D or A
  VARIANT 14: note = Y or A
GGXSGSSXXHPMXX

SEQ ID NO: 4 - protein, 17 aa, synthetic construct
  VARIANT 8: note = Y or A
  VARIANT 10: note = T or S
  VARIANT 12: note = Q or R
  VARIANT 15: note = Y or A
KSSQSLLXSXNXKNXLA

SEQ ID NO: 5 - protein, 7 aa, synthetic construct
  VARIANT 1: note = W or A
XASTRES

SEQ ID NO: 6 - protein, 9 aa, synthetic construct
  VARIANT 3: note = Y or A
  VARIANT 4: note = F or Y
  VARIANT 5: note = I or N
  VARIANT 6: note = Y or A
  VARIANT 8: note = R or Y
QQXXXXPXT

SEQ ID NO: 7 - protein, 17 aa, synthetic construct
  VARIANT 1: note = D or A
  VARIANT 5: note = D or A
  VARIANT 6: note = Y or A
  VARIANT 7: note = D or A
  VARIANT 11: note = Y or A
XINPXXXTTSXNEKFKG

SEQ ID NO: 8 - protein, 17 aa, synthetic construct
  VARIANT 8: note = Y or A
  VARIANT 15: note = Y or A
KSSQSLLXSTNQKNXLA

SEQ ID NO: 9 - protein, 9 aa, synthetic construct
  VARIANT 3: note = Y or A
  VARIANT 6: note = Y or A
QQXFIXPRT

SEQ ID NO: 10 - protein, 5 aa, synthetic construct
DYNMD

SEQ ID NO: 11 - protein, 17 aa, synthetic construct
DINPDYDTTSYNEKFKG

SEQ ID NO: 12 - protein, 14 aa, synthetic construct
GGYSGSSYYHPMDY

SEQ ID NO: 13 - protein, 17 aa, synthetic construct
KSSQSLLYSTNQKNYLA

SEQ ID NO: 14 - protein, 7 aa, synthetic construct
WASTRES

SEQ ID NO: 15 - protein, 9 aa, synthetic construct
QQYFIYPRT

SEQ ID NO: 16 - protein, 5 aa, synthetic construct
AYNMD

SEQ ID NO: 17 - protein, 5 aa, synthetic construct
DANMD

SEQ ID NO: 18 - protein, 5 aa, synthetic construct
DYNMA

SEQ ID NO: 19 - protein, 17 aa, synthetic construct
AINPDYDTTSYNEKFKG

SEQ ID NO: 20 - protein, 17 aa, synthetic construct
DINPAYDTTSYNEKFKG

SEQ ID NO: 21 - protein, 17 aa, synthetic construct
DINPDADTTSYNEKFKG

SEQ ID NO: 22 - protein, 17 aa, synthetic construct
DINPDYATTSYNEKFKG

SEQ ID NO: 23 - protein, 17 aa, synthetic construct
DINPDYDTTSANEKFKG

SEQ ID NO: 24 - protein, 14 aa, synthetic construct
GGASGSSYYHPMDY

SEQ ID NO: 25 - protein, 14 aa, synthetic construct
GGYSGSSAYHPMDY

SEQ ID NO: 26 - protein, 14 aa, synthetic construct
GGYSGSSYAHPMDY

SEQ ID NO: 27 - protein, 14 aa, synthetic construct
GGYSGSSYYHPMAY

SEQ ID NO: 28 - protein, 14 aa, synthetic construct
GGYSGSSYYHPMDA

SEQ ID NO: 29 - protein, 17 aa, synthetic construct
KSSQSLLASTNQKNYLA

SEQ ID NO: 30 - protein, 17 aa, synthetic construct
KSSQSLLYSTNQKNALA

SEQ ID NO: 31 - protein, 7 aa, synthetic construct
AASTRES

SEQ ID NO: 32 - protein, 9 aa, synthetic construct
QQAFIYPRT

SEQ ID NO: 33 - protein, 9 aa, synthetic construct
QQYFIAPRT

SEQ ID NO: 34 - protein, 17 aa, synthetic construct
DINPNYDTTSYSQKFKG

SEQ ID NO: 35 - protein, 12 aa, synthetic construct
SEAGNYYWYFDV

SEQ ID NO: 36 - protein, 17 aa, synthetic construct
KSSQSLLYSSNRKNYLA

SEQ ID NO: 37 - protein, 9 aa, synthetic construct
QQYYNYPYT

SEQ ID NO: 38 - protein, 5 aa, synthetic construct
NYWLG

SEQ ID NO: 39 - protein, 17 aa, synthetic construct
DIFPGSDNTYYNEKFKG

SEQ ID NO: 40 - protein, 9 aa, synthetic construct
SGNFYAMDY

SEQ ID NO: 41 - protein, 10 aa, synthetic construct
SASSSVNYMN

SEQ ID NO: 42 - protein, 7 aa, synthetic construct
DTSKLAS

SEQ ID NO: 43 - protein, 9 aa, synthetic construct
FQGSGYPFT

SEQ ID NO: 44 - protein, 123 aa, synthetic construct
EVQLVQSGAEVKKPGSSVKVSCKASGYIFTDYNMDWVRQAPGQGLEWIGDINPDYDTTSYNEKFKGRATLTVDKSTSTAYMELSSLRSEDTAVYYCARGGYSGSSYYHPMDYWGQGTLVTVSS

SEQ ID NO: 45 - protein, 123 aa, synthetic construct
EVQLVQSGAEVKKPGSSVKVSCKASGYIFTAYNMDWVRQAPGQGLEWIGDINPDYDTTSYNEKFKGRATLTVDKSTSTAYMELSSLRSEDTAVYYCARGGYSGSSYYHPMDYWGQGTLVTVSS

SEQ ID NO: 46 - protein, 123 aa, synthetic construct
EVQLVQSGAEVKKPGSSVKVSCKASGYIFTDANMDWVRQAPGQGLEWIGDINPDYDTTSYNEKFKGRATLTVDKSTSTAYMELSSLRSEDTAVYYCARGGYSGSSYYHPMDYWGQGTLVTVSS

SEQ ID NO: 47 - protein, 123 aa, synthetic construct
EVQLVQSGAEVKKPGSSVKVSCKASGYIFTDYNMAWVRQAPGQGLEWIGDINPDYDTTSYNEKFKGRATLTVDKSTSTAYMELSSLRSEDTAVYYCARGGYSGSSYYHPMDYWGQGTLVTVSS

SEQ ID NO: 48 - protein, 123 aa, synthetic construct
EVQLVQSGAEVKKPGSSVKVSCKASGYIFTDYNMDWVRQAPGQGLEWIGAINPDYDTTSYNEKFKGRATLTVDKSTSTAYMELSSLRSEDTAVYYCARGGYSGSSYYHPMDYWGQGTLVTVSS

SEQ ID NO: 49 - protein, 123 aa, synthetic construct
EVQLVQSGAEVKKPGSSVKVSCKASGYIFTDYNMDWVRQAPGQGLEWIGDINPAYDTTSYNEKFKGRATLTVDKSTSTAYMELSSLRSEDTAVYYCARGGYSGSSYYHPMDYWGQGTLVTVSS

SEQ ID NO: 50 - protein, 123 aa, synthetic construct
EVQLVQSGAEVKKPGSSVKVSCKASGYIFTDYNMDWVRQAPGQGLEWIGDINPDADTTSYNEKFKGRATLTVDKSTSTAYMELSSLRSEDTAVYYCARGGYSGSSYYHPMDYWGQGTLVTVSS

SEQ ID NO: 51 - protein, 123 aa, synthetic construct
EVQLVQSGAEVKKPGSSVKVSCKASGYIFTDYNMDWVRQAPGQGLEWIGDINPDYATTSYNEKFKGRATLTVDKSTSTAYMELSSLRSEDTAVYYCARGGYSGSSYYHPMDYWGQGTLVTVSS

SEQ ID NO: 52 - protein, 123 aa, synthetic construct
EVQLVQSGAEVKKPGSSVKVSCKASGYIFTDYNMDWVRQAPGQGLEWIGDINPDYDTTSANEKFKGRATLTVDKSTSTAYMELSSLRSEDTAVYYCARGGYSGSSYYHPMDYWGQGTLVTVSS

SEQ ID NO: 53 - protein, 123 aa, synthetic construct
EVQLVQSGAEVKKPGSSVKVSCKASGYIFTDYNMDWVRQAPGQGLEWIGDINPDYDTTSYNEKFKGRATLTVDKSTSTAYMELSSLRSEDTAVYYCARGGASGSSYYHPMDYWGQGTLVTVSS

SEQ ID NO: 54 - protein, 123 aa, synthetic construct
EVQLVQSGAEVKKPGSSVKVSCKASGYIFTDYNMDWVRQAPGQGLEWIGDINPDYDTTSYNEKFKGRATLTVDKSTSTAYMELSSLRSEDTAVYYCARGGYSGSSAYHPMDYWGQGTLVTVSS

SEQ ID NO: 55 - protein, 123 aa, synthetic construct
EVQLVQSGAEVKKPGSSVKVSCKASGYIFTDYNMDWVRQAPGQGLEWIGDINPDYDTTSYNEKFKGRATLTVDKSTSTAYMELSSLRSEDTAVYYCARGGYSGSSYAHPMDYWGQGTLVTVSS

SEQ ID NO: 56 - protein, 123 aa, synthetic construct
EVQLVQSGAEVKKPGSSVKVSCKASGYIFTDYNMDWVRQAPGQGLEWIGDINPDYDTTSYNEKFKGRATLTVDKSTSTAYMELSSLRSEDTAVYYCARGGYSGSSYYHPMAYWGQGTLVTVSS

SEQ ID NO: 57 - protein, 123 aa, synthetic construct
EVQLVQSGAEVKKPGSSVKVSCKASGYIFTDYNMDWVRQAPGQGLEWIGDINPDYDTTSYNEKFKGRATLTVDKSTSTAYMELSSLRSEDTAVYYCARGGYSGSSYYHPMDAWGQGTLVTVSS

SEQ ID NO: 58 - protein, 113 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGQGTKVEIK

SEQ ID NO: 59 - protein, 113 aa, synthetic construct
DIQMTQSPSSVSASVGDRVTITCKSSQSLLASTNQKNYLAWYQQKPGKAPKLLIYWASTRESGVPSRFSGSGSGTDFTLTISSLQPEDFATYYCQQYFIYPRTFGQGTKVEIK

SEQ ID NO: 60 - protein, 113 aa, synthetic construct
DIQMTQSPSSVSASVGDRVTITCKSSQSLLYSTNQKNALAWYQQKPGKAPKLLIYWASTRESGVPSRFSGSGSGTDFTLTISSLQPEDFATYYCQQYFIYPRTFGQGTKVEIK

SEQ ID NO: 61 - protein, 113 aa, synthetic construct
DIQMTQSPSSVSASVGDRVTITCKSSQSLLYSTNQKNYLAWYQQKPGKAPKLLIAWASTRESGVPSRFSGSGSGTDFTLTISSLQPEDFATYYCQQYFIYPRTFGQGTKVEIK

SEQ ID NO: 62 - protein, 113 aa, synthetic construct
DIQMTQSPSSVSASVGDRVTITCKSSQSLLYSTNQKNYLAWYQQKPGKAPKLLIYAASTRESGVPSRFSGSGSGTDFTLTISSLQPEDFATYYCQQYFIYPRTFGQGTKVEIK

SEQ ID NO: 63 - protein, 113 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQAFIYPRTFGQGTKVEIK

SEQ ID NO: 64 - protein, 113 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIAPRTFGQGTKVEIK

SEQ ID NO: 65 - protein, 123 aa, synthetic construct
EVQLQQFGVELVKPGASVKISCKASGYIFTDYNMDWVKQSHGKSLEWIGDINPDYDTTSYNEKFKGKATLTVDKSSSTAYMELRSLTSEDTAVYYCARGGYSGSSYYHPMDYWGQGTSVTVSS

SEQ ID NO: 66 - protein, 113 aa, synthetic construct
DIVMSQSPSSLAVSVGEKITMSCKSSQSLLYSTNQKNYLAWYQQKPGQSPELLIYWASTRESGVPDRFTGSGSGTDFTLTISNVRAEDLAVYYCQQYFIYPRTFGGGTKLEIK

SEQ ID NO: 67 - protein, 121 aa, synthetic construct
EVQLQQFGAELVKPGTSVKISCKASGYTFTDYNMDWVKQGHGKGLEWIGDINPNYDTTSYSQKFKGKATLTVDKSSSTAYMELRSLTSEDTAVYYCARSEAGNYYWYFDVWGAGTTVTVSS

SEQ ID NO: 68 - protein, 113 aa, synthetic construct
DIVMSQSPSSLAVSVGEKVTMSCKSSQSLLYSSNRKNYLAWYRQKPGQSPKLLIYWASTRESGVPDRFTGSGSGTDFTLTISSVKAEDLAVYSCQQYYNYPYTFGGGTKLEIK

SEQ ID NO: 69 - protein, 118 aa, synthetic construct
QVQLQQSGAELVRPGTSVKISCKASGYAFTNYWLGWVKQRPGHGLEWIGDIFPGSDNTYYNEKFKGKATLTADKSSSTAYMQLSSLASEDSAVYFCARSGNFYAMDYWGQGTSVTVSS

SEQ ID NO: 70 - protein, 106 aa, synthetic construct
ENVLTQSPAIMSASPGEKVTMTCSASSSVNYMNWYQQKSSTFPKLWIYDTSKLASGVPGRFSGSGSGKFYSLTISSMEAEDVATYYCFQGSGYPFTFGSGTKLEIK

SEQ ID NO: 71 - protein, 703 aa, synthetic construct
HHHHHHHHHHGGSQYIKANSKFIGITELGGSCKGVEPKTECERLAGTESPVREEPGEDFPAARRLYWDDLKRKLSEKLDSTDFTSTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 72 - protein, 700 aa, synthetic construct
HHHHHHHHHHSGGGGSGGGGSGGGGSKRVEQKEESVKLAETEETDKSETMETEDVPTSSRLYWADLKTLLSEKLNSIEFADTIKQLSQNTYTPREAGSQKDESLAYYIENQFHEFKFSKVWRDEHYVKIQVKSSIGQNMVTIVQSNGNLDPVESPEGYVAFSKPTEVSGKLVHANFGTKKDFEELSYSVNGSLVIVRAGEITFAEKVANAQSFNAIGVLIYMDKNKFPVVEADLALFGHAHLGTGDPYTPGFPSFNHTQFPPSQSSGLPNIPVQTISRAAAEKLFGKMEGSCPARWNIDSSCKLELSQNQNVKLIVKNVLKERRILNIFGVIKGYEEPDRYVVVGAQRDALGAGVAAKSSVGTGLLLKLAQVFSDMISKDGFRPSRSIIFASWTAGDFGAVGATEWLEGYLSSLHLKAFTYINLDKVVLGTSNFKVSASPLLYTLMGKIMQDVKHPVDGKSLYRDSNWISKVEKLSFDNAAYPFLAYSGIPAVSFCFCEDADYPYLGTRLDTYEALTQKVPQLNQMVRTAAEVAGQLIIKLTHDVELNLDYEMYNSKLLSFMKDLNQFKTDIRDMGLSLQWLYSARGDYFRATSRLTTDFHNAEKTNRFVMREINDRIMKVEYHFLSPYVSPRESPFRHIFWGSGSHTLSALVENLKLRQKNITAFNETLFRNQLALATWTIQGVANALSGDIWNIDNEF

SEQ ID NO: 73 - protein, 697 aa, synthetic construct
HHHHHHHHHHSGGGGSGGGGSGGGGSKGVEPKTESERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 74 - protein, 689 aa, synthetic construct
HHHHHHHHHHENLYFQPCKGVEPKTECERLAGTESPVREEPGEDFPAARRLYWDDLKRKLSEKLDSTDFTSTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 75 - protein, 694 aa, synthetic construct
HHHHHHHHHHALEVLFQGPCKRVEQKEECVKLAETEETDKSETMETEDVPTSSRLYWADLKTLLSEKLNSIEFADTIKQLSQNTYTPREAGSQKDESLAYYIENQFHEFKFSKVWRDEHYVKIQVKSSIGQNMVTIVQSNGNLDPVESPEGYVAFSKPTEVSGKLVHANFGTKKDFEELSYSVNGSLVIVRAGEITFAEKVANAQSFNAIGVLIYMDKNKFPVVEADLALFGHAHLGTGDPYTPGFPSFNHTQFPPSQSSGLPNIPVQTISRAAAEKLFGKMEGSCPARWNIDSSCKLELSQNQNVKLIVKNVLKERRILNIFGVIKGYEEPDRYVVVGAQRDALGAGVAAKSSVGTGLLLKLAQVFSDMISKDGFRPSRSIIFASWTAGDFGAVGATEWLEGYLSSLHLKAFTYINLDKVVLGTSNFKVSASPLLYTLMGKIMQDVKHPVDGKSLYRDSNWISKVEKLSFDNAAYPFLAYSGIPAVSFCFCEDADYPYLGTRLDTYEALTQKVPQLNQMVRTAAEVAGQLIIKLTHDVELNLDYEMYNSKLLSFMKDLNQFKTDIRDMGLSLQWLYSARGDYFRATSRLTTDFHNAEKTNRFVMREINDRIMKVEYHFLSPYVSPRESPFRHIFWGSGSHTLSALVENLKLRQKNITAFNETLFRNQLALATWTIQGVANALSGDIWNIDNEF

SEQ ID NO: 76 - protein, 700 aa, synthetic construct
HHHHHHHHHHSGGGGSGGGGSGGGGSKRVEQKEESVKLAETEETDKSETMETEDVPTSSRLYWADLKTLLSEKLNSIEFADTIKQLSQNTYTPREAGSQKDESLAYYIENQFHEFKFSKVWRDEHYVKIQVKSSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKERRILNIFGVIKGYEEPDRYVVVGAQRDALGAGVAAKSSVGTGLLLKLAQVFSDMISKDGFRPSRSIIFASWTAGDFGAVGATEWLEGYLSSLHLKAFTYINLDKVVLGTSNFKVSASPLLYTLMGKIMQDVKHPVDGKSLYRDSNWISKVEKLSFDNAAYPFLAYSGIPAVSFCFCEDADYPYLGTRLDTYEALTQKVPQLNQMVRTAAEVAGQLIIKLTHDVELNLDYEMYNSKLLSFMKDLNQFKTDIRDMGLSLQWLYSARGDYFRATSRLTTDFHNAEKTNRFVMREINDRIMKVEYHFLSPYVSPRESPFRHIFWGSGSHTLSALVENLKLRQKNITAFNETLFRNQLALATWTIQGVANALSGDIWNIDNEF

SEQ ID NO: 77 - protein, 700 aa, synthetic construct
HHHHHHHHHHSGGGGSGGGGSGGGGSKRVEQKEESVKLAETEETDKSETMETEDVPTSSRLYWADLKTLLSEKLNSIEFADTIKQLSQNTYTPREAGSQKDESLAYYIENQFHEFKFSKVWRDEHYVKIQVKSSIGQNMVTIVQSNGNLDPVESPEGYVAFSKPTEVSGKLVHANFGTKKDFEELSYSVNGSLVIVRAGEITFAEKVANAQSFNAIGVLIYMDKNKFPVVEADLALFGHAHLGTGDPYTPGFPSFNHTQFPPSQSSGLPNIPVQTISRAAAEKLFGKMEGSCPARWNIDSSCKLELSQNQNVKLIVKNVLKERRILNIFGVIKGYEEPDRYVVVGAQRDALGAGVAAKSSVGTGLLLKLAQVFSDMISKDGFRPSRSIIFASWTAGDFGAVGATEWLEGYLSSLHLKAFTYINLDKVVLGTSNFKVSASPLLYTLMGKIMQDVKHPVDGKSLYRDSNWISKVEKLSFDNAAYPFLAYSGIPAVSFCFCEDADYPYLGTRLDTYEALTQKVPQLNQMVRTAAEVAGQLIIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 78 - protein, 697 aa, synthetic construct
HHHHHHHHHHSGGGGSGGGGSGGGGSKGVEPKTESERLAGTESPVREEPGEDFPAARRLYWDDLKRKLSEKLDSTDFTGTIKLLNENSYVPREAGSQKDENLALYVENQFREFKLSKVWRDQHFVKIQVKDSIGQNMVTIVQSNGNLDPVESPEGYVAFSKPTEVSGKLVHANFGTKKDFEELSYSVNGSLVIVRAGEITFAEKVANAQSFNAIGVLIYMDKNKFPVVEADLALFGHAHLGTGDPYTPGFPSFNHTQFPPSQSSGLPNIPVQTISRAAAEKLFGKMEGSCPARWNIDSSCKLELSQNQNVKLIVKNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYEMYNSKLLSFMKDLNQFKTDIRDMGLSLQWLYSARGDYFRATSRLTTDFHNAEKTNRFVMREINDRIMKVEYHFLSPYVSPRESPFRHIFWGSGSHTLSALVENLKLRQKNITAFNETLFRNQLALATWTIQGVANALSGDIWNIDNEF

SEQ ID NO: 79 - protein, 251 aa, synthetic construct
EVQLQQFGVELVKPGASVKISCKASGYIFTDYNMDWVKQSHGKSLEWIGDINPDYDTTSYNEKFKGKATLTVDKSSSTAYMELRSLTSEDTAVYYCARGGYSGSSYYHPMDYWGQGTSVTVSSGGGGSGGGGSGGGGSDIVMSQSPSSLAVSVGEKITMSCKSSQSLLYSTNQKNYLAWYQQKPGQSPELLIYWASTRESGVPDRFTGSGSGTDFTLTISNVRAEDLAVYYCQQYFIYPRTFGGGTKLEIK

SEQ ID NO: 80 - protein, 251 aa, synthetic construct
EVQLVQSGAEVKKPGSSVKVSCKASGYIFTDYNMDWVRQAPGQGLEWIGDINPDYDTTSYNEKFKGRATLTVDKSTSTAYMELSSLRSEDTAVYYCARGGYSGSSYYHPMDYWGQGTLVTVSSGGGGSGGGGSGGGGSDIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGQGTKVEIK

SEQ ID NO: 81 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 83 - protein, 483 aa, synthetic construct
EVQLQQFGVELVKPGASVKISCKASGYIFTDYNMDWVKQSHGKSLEWIGDINPDYDTTSYNEKFKGKATLTVDKSSSTAYMELRSLTSEDTAVYYCARGGYSGSSYYHPMDYWGQGTSVTVSSGGGGSGGGGSGGGGSDIVMSQSPSSLAVSVGEKITMSCKSSQSLLYSTNQKNYLAWYQQKPGQSPELLIYWASTRESGVPDRFTGSGSGTDFTLTISNVRAEDLAVYYCQQYFIYPRTFGGGTKLEIKGGGGSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 483 aa, synthetic construct
EVQLVQSGAEVKKPGSSVKVSCKASGYIFTDYNMDWVRQAPGQGLEWIGDINPDYDTTSYNEKFKGRATLTVDKSTSTAYMELSSLRSEDTAVYYCARGGYSGSSYYHPMDYWGQGTLVTVSSGGGGSGGGGSGGGGSDIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGQGTKVEIKGGGGSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 760 aa, synthetic construct
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 86 - protein, 664 aa, synthetic construct
HHHHHHHHHHDYKDDDDKENLYFQGLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 87 - protein, 664 aa, synthetic construct
HHHHHHHHHHDYKDDDDKENLYFQGLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVSNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGLSLQWLYSARGDFFRATSRLTTDFRNAEKRDKFVMKKLNDRVMRVEYYFLSPYVSPKESPFRHVFWGSGSHTLSALLESLKLRRQNNSAFNETLFRNQLALATWTIQGAANALSGDVWDIDNEF

SEQ ID NO: 88 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDTAVYYCARGGYSGSSYYHPMDYWGQGTLVTVSS

SEQ ID NO: 89 - protein, 123 aa, synthetic construct
EVQLVQSGAEVKKPGSSVKVSCKASGYIFTAYNMDWVRQAPGQCLEWIGDINPDYDTTSYNEKFKGRATLTVDKSTSTAYMELSSLRSEDTAVYYCARGGYSGSSYYHPMDYWGQGTLVTVSS

SEQ ID NO: 90 - protein, 123 aa, synthetic construct
EVQLVQSGAEVKKPGSSVKVSCKASGYIFTDANMDWVRQAPGQCLEWIGDINPDYDTTSYNEKFKGRATLTVDKSTSTAYMELSSLRSEDTAVYYCARGGYSGSSYYHPMDYWGQGTLVTVSS

SEQ ID NO: 91 - protein, 123 aa, synthetic construct
EVQLVQSGAEVKKPGSSVKVSCKASGYIFTDYNMAWVRQAPGQCLEWIGDINPDYDTTSYNEKFKGRATLTVDKSTSTAYMELSSLRSEDTAVYYCARGGYSGSSYYHPMDYWGQGTLVTVSS

SEQ ID NO: 92 - protein, 123 aa, synthetic construct
EVQLVQSGAEVKKPGSSVKVSCKASGYIFTDYNMDWVRQAPGQCLEWIGAINPDYDTTSYNEKFKGRATLTVDKSTSTAYMELSSLRSEDTAVYYCARGGYSGSSYYHPMDYWGQGTLVTVSS

SEQ ID NO: 93 - protein, 123 aa, synthetic construct
EVQLVQSGAEVKKPGSSVKVSCKASGYIFTDYNMDWVRQAPGQCLEWIGDINPAYDTTSYNEKFKGRATLTVDKSTSTAYMELSSLRSEDTAVYYCARGGYSGSSYYHPMDYWGQGTLVTVSS

SEQ ID NO: 94 - protein, 123 aa, synthetic construct
EVQLVQSGAEVKKPGSSVKVSCKASGYIFTDYNMDWVRQAPGQCLEWIGDINPDADTTSYNEKFKGRATLTVDKSTSTAYMELSSLRSEDTAVYYCARGGYSGSSYYHPMDYWGQGTLVTVSS

SEQ ID NO: 95 - protein, 123 aa, synthetic construct
EVQLVQSGAEVKKPGSSVKVSCKASGYIFTDYNMDWVRQAPGQCLEWIGDINPDYATTSYNEKFKGRATLTVDKSTSTAYMELSSLRSEDTAVYYCARGGYSGSSYYHPMDYWGQGTLVTVSS

SEQ ID NO: 96 - protein, 123 aa, synthetic construct
EVQLVQSGAEVKKPGSSVKVSCKASGYIFTDYNMDWVRQAPGQCLEWIGDINPDYDTTSANEKFKGRATLTVDKSTSTAYMELSSLRSEDTAVYYCARGGYSGSSYYHPMDYWGQGTLVTVSS

SEQ ID NO: 97 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDTAVYYCARGGASGSSYYHPMDYWGQGTLVTVSS

SEQ ID NO: 98 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDTAVYYCARGGYSGSSAYHPMDYWGQGTLVTVSS

SEQ ID NO: 99 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDTAVYYCARGGYSGSSYAHPMDYWGQGTLVTVSS

SEQ ID NO: 100 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDTAVYYCARGGYSGSSYYHPMAYWGQGTLVTVSS

SEQ ID NO: 101 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDTAVYYCARGGYSGSSYYHPMDAWGQGTLVTVSS

SEQ ID NO: 102 - protein, 123 aa, synthetic construct
EVQLQQFGVELVKPGASVKISCKASGYIFTDYNMDWVKQSHGKCLEWIGDINPDYDTTSYNEKFKGKATLTVDKSSSTAYMELRSLTSEDTAVYYCARGGYSGSSYYHPMDYWGQGTSVTVSS

SEQ ID NO: 103 - protein, 121 aa, synthetic construct
EVQLQQFGAELVKPGTSVKISCKASGYTFTDYNMDWVKQGHGKCLEWIGDINPNYDTTSYSQKFKGKATLTVDKSSSTAYMELRSLTSEDTAVYYCARSEAGNYYWYFDVWGAGTTVTVSS

SEQ ID NO: 104 - protein, 118 aa, synthetic construct
QVQLQQSGAELVRPGTSVKISCKASGYAFTNYWLGWVKQRPGHCLEWIGDIFPGSDNTYYNEKFKGKATLTADKSSSTAYMQLSSLASEDSAVYFCARSGNFYAMDYWGQGTSVTVSS

SEQ ID NO: 105 - protein, 113 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGCGTKVEIK

SEQ ID NO: 106 - protein, 113 aa, synthetic construct
DIQMTQSPSSVSASVGDRVTITCKSSQSLLASTNQKNYLAWYQQKPGKAPKLLIYWASTRESGVPSRFSGSGSGTDFTLTISSLQPEDFATYYCQQYFIYPRTFGCGTKVEIK

SEQ ID NO: 107 - protein, 113 aa, synthetic construct
DIQMTQSPSSVSASVGDRVTITCKSSQSLLYSTNQKNALAWYQQKPGKAPKLLIYWASTRESGVPSRFSGSGSGTDFTLTISSLQPEDFATYYCQQYFIYPRTFGCGTKVEIK

SEQ ID NO: 108 - protein, 113 aa, synthetic construct
DIQMTQSPSSVSASVGDRVTITCKSSQSLLYSTNQKNYLAWYQQKPGKAPKLLIAWASTRESGVPSRFSGSGSGTDFTLTISSLQPEDFATYYCQQYFIYPRTFGCGTKVEIK

SEQ ID NO: 109 - protein, 113 aa, synthetic construct
DIQMTQSPSSVSASVGDRVTITCKSSQSLLYSTNQKNYLAWYQQKPGKAPKLLIYAASTRESGVPSRFSGSGSGTDFTLTISSLQPEDFATYYCQQYFIYPRTFGCGTKVEIK

SEQ ID NO: 110 - protein, 113 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQAFIYPRTFGCGTKVEIK

SEQ ID NO: 111 - protein, 113 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIAPRTFGCGTKVEIK

SEQ ID NO: 112 - protein, 113 aa, synthetic construct
DIVMSQSPSSLAVSVGEKITMSCKSSQSLLYSTNQKNYLAWYQQKPGQSPELLIYWASTRESGVPDRFTGSGSGTDFTLTISNVRAEDLAVYYCQQYFIYPRTFGCGTKLEIK

SEQ ID NO: 113 - protein, 113 aa, synthetic construct
DIVMSQSPSSLAVSVGEKVTMSCKSSQSLLYSSNRKNYLAWYRQKPGQSPKLLIYWASTRESGVPDRFTGSGSGTDFTLTISSVKAEDLAVYSCQQYYNYPYTFGCGTKLEIK

SEQ ID NO: 114 - protein, 106 aa, synthetic construct
ENVLTQSPAIMSASPGEKVTMTCSASSSVNYMNWYQQKSSTFPKLWIYDTSKLASGVPGRFSGSGSGKFYSLTISSMEAEDVATYYCFQGSGYPFTFGCGTKLEIK

SEQ ID NO: 115 - protein, 251 aa, synthetic construct
EVQLVQSGAEVKKPGSSVKVSCKASGYIFTDYNMDWVRQAPGQGLEWIGDINPDADTTSYNEKFKGRATLTVDKSTSTAYMELSSLRSEDTAVYYCARGGYSGSSYYHPMDYWGQGTLVTVSSGGGGSGGGGSGGGGSDIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGQGTKVEIK

SEQ ID NO: 116 - protein, 251 aa, synthetic construct
EVQLVQSGAEVKKPGSSVKVSCKASGYIFTDYNMDWVRQAPGQCLEWIGDINPDADTTSYNEKFKGRATLTVDKSTSTAYMELSSLRSEDTAVYYCARGGYSGSSYYHPMDYWGQGTLVTVSSGGGGSGGGGSGGGGSDIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGCGTKVEIK

SEQ ID NO: 117 - protein, 251 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGQGTKVEIKGGGGSGGGGSGGGGSEVQLVQSGAEVKKPGSSVKVSCKASGYIFTDYNMDWVRQAPGQGLEWIGDINPDADTTSYNEKFKGRATLTVDKSTSTAYMELSSLRSEDTAVYYCARGGYSGSSYYHPMDYWGQGTLVTVSS

SEQ ID NO: 118 - protein, 256 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGCGTKVEIKGGGGSGGGGSGGGGSGGGGSEVQLVQSGAEVKKPGSSVKVSCKASGYIFTDYNMDWVRQAPGQCLEWIGDINPDADTTSYNEKFKGRATLTVDKSTSTAYMELSSLRSEDTAVYYCARGGYSGSSYYHPMDYWGQGTLVTVSS

SEQ ID NO: 119 - protein, 251 aa, synthetic construct
EVQLVQSGAEVKKPGSSVKVSCKASGYIFTDYNMDWVRQAPGQGLEWIGDINPDYDTTSYNEKFKGRATLTVDKSTSTAYMELSSLRSEDTAVYYCARGGYSGSSYYHPMDYWGQGTLVTVSSGGGGSGGGGSGGGGSDIQMTQSPSSVSASVGDRVTITCKSSQSLLASTNQKNYLAWYQQKPGKAPKLLIYWASTRESGVPSRFSGSGSGTDFTLTISSLQPEDFATYYCQQYFIYPRTFGQGTKVEIK

SEQ ID NO: 120 - protein, 256 aa, synthetic construct
DIQMTQSPSSVSASVGDRVTITCKSSQSLLASTNQKNYLAWYQQKPGKAPKLLIYWASTRESGVPSRFSGSGSGTDFTLTISSLQPEDFATYYCQQYFIYPRTFGCGTKVEIKGGGGSGGGGSGGGGSGGGGSEVQLVQSGAEVKKPGSSVKVSCKASGYIFTDYNMDWVRQAPGQCLEWIGDINPDYDTTSYNEKFKGRATLTVDKSTSTAYMELSSLRSEDTAVYYCARGGYSGSSYYHPMDYWGQGTLVTVSS

SEQ ID NO: 121 - protein, 256 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGCGTKVEIKGGGGSGGGGSGGGGSGGGGSEVQLVQSGAEVKKPGSSVKVSCKASGYIFTDYNMDWVRQAPGQCLEWIGDINPDYDTTSYNEKFKGRATLTVDKSTSTAYMELSSLRSEDTAVYYCARGGYSGSSYYHPMDYWGQGTLVTVSS

SEQ ID NO: 122 - protein, 26 aa, synthetic construct
HHHHHHGGGGSGGGGSGGGGSGGGGS

SEQ ID NO: 123 - protein, 41 aa, synthetic construct
HHHHHHGLNDIFEAQKIEWHEGGGGSGGGGSGGGGSGGGGS

SEQ ID NO: 124 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDSAVYYCARGGYSGSSYYHPMDYWGQGTLVTVKS

SEQ ID NO: 125 - protein, 123 aa, synthetic construct
EVQLVQSGAEVKKPGSSVKVSCKASGYIFTAYNMDWVRQAPGQCLEWIGDINPDYDTTSYNEKFKGRATLTVDKSTSTAYMELSSLRSEDSAVYYCARGGYSGSSYYHPMDYWGQGTLVTVKS

SEQ ID NO: 126 - protein, 123 aa, synthetic construct
EVQLVQSGAEVKKPGSSVKVSCKASGYIFTDANMDWVRQAPGQCLEWIGDINPDYDTTSYNEKFKGRATLTVDKSTSTAYMELSSLRSEDSAVYYCARGGYSGSSYYHPMDYWGQGTLVTVKS

SEQ ID NO: 127 - protein, 123 aa, synthetic construct
EVQLVQSGAEVKKPGSSVKVSCKASGYIFTDYNMAWVRQAPGQCLEWIGDINPDYDTTSYNEKFKGRATLTVDKSTSTAYMELSSLRSEDSAVYYCARGGYSGSSYYHPMDYWGQGTLVTVKS

SEQ ID NO: 128 - protein, 123 aa, synthetic construct
EVQLVQSGAEVKKPGSSVKVSCKASGYIFTDYNMDWVRQAPGQCLEWIGAINPDYDTTSYNEKFKGRATLTVDKSTSTAYMELSSLRSEDSAVYYCARGGYSGSSYYHPMDYWGQGTLVTVKS

SEQ ID NO: 129 - protein, 123 aa, synthetic construct
EVQLVQSGAEVKKPGSSVKVSCKASGYIFTDYNMDWVRQAPGQCLEWIGDINPAYDTTSYNEKFKGRATLTVDKSTSTAYMELSSLRSEDSAVYYCARGGYSGSSYYHPMDYWGQGTLVTVKS

SEQ ID NO: 130 - protein, 123 aa, synthetic construct
EVQLVQSGAEVKKPGSSVKVSCKASGYIFTDYNMDWVRQAPGQCLEWIGDINPDADTTSYNEKFKGRATLTVDKSTSTAYMELSSLRSEDSAVYYCARGGYSGSSYYHPMDYWGQGTLVTVKS

SEQ ID NO: 131 - protein, 123 aa, synthetic construct
EVQLVQSGAEVKKPGSSVKVSCKASGYIFTDYNMDWVRQAPGQCLEWIGDINPDYATTSYNEKFKGRATLTVDKSTSTAYMELSSLRSEDSAVYYCARGGYSGSSYYHPMDYWGQGTLVTVKS

SEQ ID NO: 132 - protein, 123 aa, synthetic construct
EVQLVQSGAEVKKPGSSVKVSCKASGYIFTDYNMDWVRQAPGQCLEWIGDINPDYDTTSANEKFKGRATLTVDKSTSTAYMELSSLRSEDSAVYYCARGGYSGSSYYHPMDYWGQGTLVTVKS

SEQ ID NO: 133 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDSAVYYCARGGASGSSYYHPMDYWGQGTLVTVKS

SEQ ID NO: 134 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDSAVYYCARGGYSGSSAYHPMDYWGQGTLVTVKS

SEQ ID NO: 135 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDSAVYYCARGGYSGSSYAHPMDYWGQGTLVTVKS

SEQ ID NO: 136 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDSAVYYCARGGYSGSSYYHPMAYWGQGTLVTVKS

SEQ ID NO: 137 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDSAVYYCARGGYSGSSYYHPMDAWGQGTLVTVKS

SEQ ID NO: 138 - protein, 123 aa, synthetic construct
EVQLQQFGVELVKPGASVKISCKASGYIFTDYNMDWVKQSHGKCLEWIGDINPDYDTTSYNEKFKGKATLTVDKSSSTAYMELRSLTSEDSAVYYCARGGYSGSSYYHPMDYWGQGTSVTVKS

SEQ ID NO: 139 - protein, 121 aa, synthetic construct
EVQLQQFGAELVKPGTSVKISCKASGYTFTDYNMDWVKQGHGKCLEWIGDINPNYDTTSYSQKFKGKATLTVDKSSSTAYMELRSLTSEDSAVYYCARSEAGNYYWYFDVWGAGTTVTVKS

SEQ ID NO: 140 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDTAVYYCARGGYSGSSYYHPMDYWGQGTLVTVKS

SEQ ID NO: 141 - protein, 123 aa, synthetic construct
EVQLVQSGAEVKKPGSSVKVSCKASGYIFTAYNMDWVRQAPGQCLEWIGDINPDYDTTSYNEKFKGRATLTVDKSTSTAYMELSSLRSEDTAVYYCARGGYSGSSYYHPMDYWGQGTLVTVKS

SEQ ID NO: 142 - protein, 123 aa, synthetic construct
EVQLVQSGAEVKKPGSSVKVSCKASGYIFTDANMDWVRQAPGQCLEWIGDINPDYDTTSYNEKFKGRATLTVDKSTSTAYMELSSLRSEDTAVYYCARGGYSGSSYYHPMDYWGQGTLVTVKS

SEQ ID NO: 143 - protein, 123 aa, synthetic construct
EVQLVQSGAEVKKPGSSVKVSCKASGYIFTDYNMAWVRQAPGQCLEWIGDINPDYDTTSYNEKFKGRATLTVDKSTSTAYMELSSLRSEDTAVYYCARGGYSGSSYYHPMDYWGQGTLVTVKS

SEQ ID NO: 144 - protein, 123 aa, synthetic construct
EVQLVQSGAEVKKPGSSVKVSCKASGYIFTDYNMDWVRQAPGQCLEWIGAINPDYDTTSYNEKFKGRATLTVDKSTSTAYMELSSLRSEDTAVYYCARGGYSGSSYYHPMDYWGQGTLVTVKS

SEQ ID NO: 145 - protein, 123 aa, synthetic construct
EVQLVQSGAEVKKPGSSVKVSCKASGYIFTDYNMDWVRQAPGQCLEWIGDINPAYDTTSYNEKFKGRATLTVDKSTSTAYMELSSLRSEDTAVYYCARGGYSGSSYYHPMDYWGQGTLVTVKS

SEQ ID NO: 146 - protein, 123 aa, synthetic construct
EVQLVQSGAEVKKPGSSVKVSCKASGYIFTDYNMDWVRQAPGQCLEWIGDINPDADTTSYNEKFKGRATLTVDKSTSTAYMELSSLRSEDTAVYYCARGGYSGSSYYHPMDYWGQGTLVTVKS

SEQ ID NO: 147 - protein, 123 aa, synthetic construct
EVQLVQSGAEVKKPGSSVKVSCKASGYIFTDYNMDWVRQAPGQCLEWIGDINPDYATTSYNEKFKGRATLTVDKSTSTAYMELSSLRSEDTAVYYCARGGYSGSSYYHPMDYWGQGTLVTVKS

SEQ ID NO: 148 - protein, 123 aa, synthetic construct
EVQLVQSGAEVKKPGSSVKVSCKASGYIFTDYNMDWVRQAPGQCLEWIGDINPDYDTTSANEKFKGRATLTVDKSTSTAYMELSSLRSEDTAVYYCARGGYSGSSYYHPMDYWGQGTLVTVKS

SEQ ID NO: 149 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDTAVYYCARGGASGSSYYHPMDYWGQGTLVTVKS

SEQ ID NO: 150 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDTAVYYCARGGYSGSSAYHPMDYWGQGTLVTVKS

SEQ ID NO: 151 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDTAVYYCARGGYSGSSYAHPMDYWGQGTLVTVKS

SEQ ID NO: 152 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDTAVYYCARGGYSGSSYYHPMAYWGQGTLVTVKS

SEQ ID NO: 153 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDTAVYYCARGGYSGSSYYHPMDAWGQGTLVTVKS

SEQ ID NO: 154 - protein, 123 aa, synthetic construct
EVQLQQFGVELVKPGASVKISCKASGYIFTDYNMDWVKQSHGKCLEWIGDINPDYDTTSYNEKFKGKATLTVDKSSSTAYMELRSLTSEDTAVYYCARGGYSGSSYYHPMDYWGQGTSVTVKS

SEQ ID NO: 155 - protein, 121 aa, synthetic construct
EVQLQQFGAELVKPGTSVKISCKASGYTFTDYNMDWVKQGHGKCLEWIGDINPNYDTTSYSQKFKGKATLTVDKSSSTAYMELRSLTSEDTAVYYCARSEAGNYYWYFDVWGAGTTVTVKS

SEQ ID NO: 156 - protein, 118 aa, synthetic construct
QVQLQQSGAELVRPGTSVKISCKASGYAFTNYWLGWVKQRPGHCLEWIGDIFPGSDNTYYNEKFKGKATLTADKSSSTAYMQLSSLASEDSAVYFCARSGNFYAMDYWGQGTSVTVKS

SEQ ID NO: 157 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDTAVYYCARGGYSGSSYYHPMDYWGQGTLVKVSS

SEQ ID NO: 158 - protein, 123 aa, synthetic construct
EVQLVQSGAEVKKPGSSVKVSCKASGYIFTAYNMDWVRQAPGQCLEWIGDINPDYDTTSYNEKFKGRATLTVDKSTSTAYMELSSLRSEDTAVYYCARGGYSGSSYYHPMDYWGQGTLVKVSS

SEQ ID NO: 159 - protein, 123 aa, synthetic construct
EVQLVQSGAEVKKPGSSVKVSCKASGYIFTDANMDWVRQAPGQCLEWIGDINPDYDTTSYNEKFKGRATLTVDKSTSTAYMELSSLRSEDTAVYYCARGGYSGSSYYHPMDYWGQGTLVKVSS

SEQ ID NO: 160 - protein, 123 aa, synthetic construct
EVQLVQSGAEVKKPGSSVKVSCKASGYIFTDYNMAWVRQAPGQCLEWIGDINPDYDTTSYNEKFKGRATLTVDKSTSTAYMELSSLRSEDTAVYYCARGGYSGSSYYHPMDYWGQGTLVKVSS

SEQ ID NO: 161 - protein, 123 aa, synthetic construct
EVQLVQSGAEVKKPGSSVKVSCKASGYIFTDYNMDWVRQAPGQCLEWIGAINPDYDTTSYNEKFKGRATLTVDKSTSTAYMELSSLRSEDTAVYYCARGGYSGSSYYHPMDYWGQGTLVKVSS

SEQ ID NO: 162 - protein, 123 aa, synthetic construct
EVQLVQSGAEVKKPGSSVKVSCKASGYIFTDYNMDWVRQAPGQCLEWIGDINPAYDTTSYNEKFKGRATLTVDKSTSTAYMELSSLRSEDTAVYYCARGGYSGSSYYHPMDYWGQGTLVKVSS

SEQ ID NO: 163 - protein, 123 aa, synthetic construct
EVQLVQSGAEVKKPGSSVKVSCKASGYIFTDYNMDWVRQAPGQCLEWIGDINPDADTTSYNEKFKGRATLTVDKSTSTAYMELSSLRSEDTAVYYCARGGYSGSSYYHPMDYWGQGTLVKVSS

SEQ ID NO: 164 - protein, 123 aa, synthetic construct
EVQLVQSGAEVKKPGSSVKVSCKASGYIFTDYNMDWVRQAPGQCLEWIGDINPDYATTSYNEKFKGRATLTVDKSTSTAYMELSSLRSEDTAVYYCARGGYSGSSYYHPMDYWGQGTLVKVSS

SEQ ID NO: 165 - protein, 123 aa, synthetic construct
EVQLVQSGAEVKKPGSSVKVSCKASGYIFTDYNMDWVRQAPGQCLEWIGDINPDYDTTSANEKFKGRATLTVDKSTSTAYMELSSLRSEDTAVYYCARGGYSGSSYYHPMDYWGQGTLVKVSS

SEQ ID NO: 166 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDTAVYYCARGGASGSSYYHPMDYWGQGTLVKVSS

SEQ ID NO: 167 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDTAVYYCARGGYSGSSAYHPMDYWGQGTLVKVSS

SEQ ID NO: 168 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDTAVYYCARGGYSGSSYAHPMDYWGQGTLVKVSS

SEQ ID NO: 169 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDTAVYYCARGGYSGSSYYHPMAYWGQGTLVKVSS

SEQ ID NO: 170 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDTAVYYCARGGYSGSSYYHPMDAWGQGTLVKVSS

SEQ ID NO: 171 - protein, 123 aa, synthetic construct
EVQLQQFGVELVKPGASVKISCKASGYIFTDYNMDWVKQSHGKCLEWIGDINPDYDTTSYNEKFKGKATLTVDKSSSTAYMELRSLTSEDTAVYYCARGGYSGSSYYHPMDYWGQGTSVKVSS

SEQ ID NO: 172 - protein, 121 aa, synthetic construct
EVQLQQFGAELVKPGTSVKISCKASGYTFTDYNMDWVKQGHGKCLEWIGDINPNYDTTSYSQKFKGKATLTVDKSSSTAYMELRSLTSEDTAVYYCARSEAGNYYWYFDVWGAGTTVKVSS

SEQ ID NO: 173 - protein, 118 aa, synthetic construct
QVQLQQSGAELVRPGTSVKISCKASGYAFTNYWLGWVKQRPGHCLEWIGDIFPGSDNTYYNEKFKGKATLTADKSSSTAYMQLSSLASEDSAVYFCARSGNFYAMDYWGQGTSVKVSS

SEQ ID NO: 174 - protein, 123 aa, synthetic construct
EVQLVQSGAERKKPGSSVKVSCKASGYIFTDYNMDWVRQAPGQCLEWIGDINPDYDTTSYNEKFKGRATLTVDKSTSTAYMELSSLRSEDTAVYYCARGGYSGSSYYHPMDYWGQGTLVKVSS

SEQ ID NO: 175 - protein, 123 aa, synthetic construct
EVQLVQSGAERKKPGSSVKVSCKASGYIFTAYNMDWVRQAPGQCLEWIGDINPDYDTTSYNEKFKGRATLTVDKSTSTAYMELSSLRSEDTAVYYCARGGYSGSSYYHPMDYWGQGTLVKVSS

SEQ ID NO: 176 - protein, 123 aa, synthetic construct
EVQLVQSGAERKKPGSSVKVSCKASGYIFTDANMDWVRQAPGQCLEWIGDINPDYDTTSYNEKFKGRATLTVDKSTSTAYMELSSLRSEDTAVYYCARGGYSGSSYYHPMDYWGQGTLVKVSS

SEQ ID NO: 177 - protein, 123 aa, synthetic construct
EVQLVQSGAERKKPGSSVKVSCKASGYIFTDYNMAWVRQAPGQCLEWIGDINPDYDTTSYNEKFKGRATLTVDKSTSTAYMELSSLRSEDTAVYYCARGGYSGSSYYHPMDYWGQGTLVKVSS

SEQ ID NO: 178 - protein, 123 aa, synthetic construct
EVQLVQSGAERKKPGSSVKVSCKASGYIFTDYNMDWVRQAPGQCLEWIGAINPDYDTTSYNEKFKGRATLTVDKSTSTAYMELSSLRSEDTAVYYCARGGYSGSSYYHPMDYWGQGTLVKVSS

SEQ ID NO: 179 - protein, 123 aa, synthetic construct
EVQLVQSGAERKKPGSSVKVSCKASGYIFTDYNMDWVRQAPGQCLEWIGDINPAYDTTSYNEKFKGRATLTVDKSTSTAYMELSSLRSEDTAVYYCARGGYSGSSYYHPMDYWGQGTLVKVSS

SEQ ID NO: 180 - protein, 123 aa, synthetic construct
EVQLVQSGAERKKPGSSVKVSCKASGYIFTDYNMDWVRQAPGQCLEWIGDINPDADTTSYNEKFKGRATLTVDKSTSTAYMELSSLRSEDTAVYYCARGGYSGSSYYHPMDYWGQGTLVKVSS

SEQ ID NO: 181 - protein, 123 aa, synthetic construct
EVQLVQSGAERKKPGSSVKVSCKASGYIFTDYNMDWVRQAPGQCLEWIGDINPDYATTSYNEKFKGRATLTVDKSTSTAYMELSSLRSEDTAVYYCARGGYSGSSYYHPMDYWGQGTLVKVSS

SEQ ID NO: 182 - protein, 123 aa, synthetic construct
EVQLVQSGAERKKPGSSVKVSCKASGYIFTDYNMDWVRQAPGQCLEWIGDINPDYDTTSANEKFKGRATLTVDKSTSTAYMELSSLRSEDTAVYYCARGGYSGSSYYHPMDYWGQGTLVKVSS

SEQ ID NO: 183 - protein, 123 aa, synthetic construct
EVQLVQSGAERKKPGSSVKVSCKASGYIFTDYNMDWVRQAPGQCLEWIGDINPDYDTTSYNEKFKGRATLTVDKSTSTAYMELSSLRSEDTAVYYCARGGASGSSYYHPMDYWGQGTLVKVSS

SEQ ID NO: 184 - protein, 123 aa, synthetic construct
EVQLVQSGAERKKPGSSVKVSCKASGYIFTDYNMDWVRQAPGQCLEWIGDINPDYDTTSYNEKFKGRATLTVDKSTSTAYMELSSLRSEDTAVYYCARGGYSGSSAYHPMDYWGQGTLVKVSS

SEQ ID NO: 185 - protein, 123 aa, synthetic construct
EVQLVQSGAERKKPGSSVKVSCKASGYIFTDYNMDWVRQAPGQCLEWIGDINPDYDTTSYNEKFKGRATLTVDKSTSTAYMELSSLRSEDTAVYYCARGGYSGSSYAHPMDYWGQGTLVKVSS

SEQ ID NO: 186 - protein, 123 aa, synthetic construct
EVQLVQSGAERKKPGSSVKVSCKASGYIFTDYNMDWVRQAPGQCLEWIGDINPDYDTTSYNEKFKGRATLTVDKSTSTAYMELSSLRSEDTAVYYCARGGYSGSSYYHPMAYWGQGTLVKVSS

SEQ ID NO: 187 - protein, 123 aa, synthetic construct
EVQLVQSGAERKKPGSSVKVSCKASGYIFTDYNMDWVRQAPGQCLEWIGDINPDYDTTSYNEKFKGRATLTVDKSTSTAYMELSSLRSEDTAVYYCARGGYSGSSYYHPMDAWGQGTLVKVSS

SEQ ID NO: 188 - protein, 123 aa, synthetic construct
EVQLQQFGVERVKPGASVKISCKASGYIFTDYNMDWVKQSHGKCLEWIGDINPDYDTTSYNEKFKGKATLTVDKSSSTAYMELRSLTSEDTAVYYCARGGYSGSSYYHPMDYWGQGTSVKVSS

SEQ ID NO: 189 - protein, 121 aa, synthetic construct
EVQLQQFGAERVKPGTSVKISCKASGYTFTDYNMDWVKQGHGKCLEWIGDINPNYDTTSYSQKFKGKATLTVDKSSSTAYMELRSLTSEDTAVYYCARSEAGNYYWYFDVWGAGTTVKVSS

SEQ ID NO: 190 - protein, 118 aa, synthetic construct
QVQLQQSGAERVRPGTSVKISCKASGYAFTNYWLGWVKQRPGHCLEWIGDIFPGSDNTYYNEKFKGKATLTADKSSSTAYMQLSSLASEDSAVYFCARSGNFYAMDYWGQGTSVKVSS

SEQ ID NO: 191 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDSAVYYCARGGYSGSSYYHPMDYWGQGTLVKVSS

SEQ ID NO: 192 - protein, 123 aa, synthetic construct
EVQLVQSGAEVKKPGSSVKVSCKASGYIFTAYNMDWVRQAPGQCLEWIGDINPDYDTTSYNEKFKGRATLTVDKSTSTAYMELSSLRSEDSAVYYCARGGYSGSSYYHPMDYWGQGTLVKVSS

SEQ ID NO: 193 - protein, 123 aa, synthetic construct
EVQLVQSGAEVKKPGSSVKVSCKASGYIFTDANMDWVRQAPGQCLEWIGDINPDYDTTSYNEKFKGRATLTVDKSTSTAYMELSSLRSEDSAVYYCARGGYSGSSYYHPMDYWGQGTLVKVSS

SEQ ID NO: 194 - protein, 123 aa, synthetic construct
EVQLVQSGAEVKKPGSSVKVSCKASGYIFTDYNMAWVRQAPGQCLEWIGDINPDYDTTSYNEKFKGRATLTVDKSTSTAYMELSSLRSEDSAVYYCARGGYSGSSYYHPMDYWGQGTLVKVSS

SEQ ID NO: 195 - protein, 123 aa, synthetic construct
EVQLVQSGAEVKKPGSSVKVSCKASGYIFTDYNMDWVRQAPGQCLEWIGAINPDYDTTSYNEKFKGRATLTVDKSTSTAYMELSSLRSEDSAVYYCARGGYSGSSYYHPMDYWGQGTLVKVSS

SEQ ID NO: 196 - protein, 123 aa, synthetic construct
EVQLVQSGAEVKKPGSSVKVSCKASGYIFTDYNMDWVRQAPGQCLEWIGDINPAYDTTSYNEKFKGRATLTVDKSTSTAYMELSSLRSEDSAVYYCARGGYSGSSYYHPMDYWGQGTLVKVSS

SEQ ID NO: 197 - protein, 123 aa, synthetic construct
EVQLVQSGAEVKKPGSSVKVSCKASGYIFTDYNMDWVRQAPGQCLEWIGDINPDADTTSYNEKFKGRATLTVDKSTSTAYMELSSLRSEDSAVYYCARGGYSGSSYYHPMDYWGQGTLVKVSS

SEQ ID NO: 198 - protein, 123 aa, synthetic construct
EVQLVQSGAEVKKPGSSVKVSCKASGYIFTDYNMDWVRQAPGQCLEWIGDINPDYATTSYNEKFKGRATLTVDKSTSTAYMELSSLRSEDSAVYYCARGGYSGSSYYHPMDYWGQGTLVKVSS

SEQ ID NO: 199 - protein, 123 aa, synthetic construct
EVQLVQSGAEVKKPGSSVKVSCKASGYIFTDYNMDWVRQAPGQCLEWIGDINPDYDTTSANEKFKGRATLTVDKSTSTAYMELSSLRSEDSAVYYCARGGYSGSSYYHPMDYWGQGTLVKVSS

SEQ ID NO: 200 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDSAVYYCARGGASGSSYYHPMDYWGQGTLVKVSS

SEQ ID NO: 201 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDSAVYYCARGGYSGSSAYHPMDYWGQGTLVKVSS

SEQ ID NO: 202 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDSAVYYCARGGYSGSSYAHPMDYWGQGTLVKVSS

SEQ ID NO: 203 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDSAVYYCARGGYSGSSYYHPMAYWGQGTLVKVSS

SEQ ID NO: 204 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDSAVYYCARGGYSGSSYYHPMDAWGQGTLVKVSS

SEQ ID NO: 205 - protein, 123 aa, synthetic construct
EVQLQQFGVELVKPGASVKISCKASGYIFTDYNMDWVKQSHGKCLEWIGDINPDYDTTSYNEKFKGKATLTVDKSSSTAYMELRSLTSEDSAVYYCARGGYSGSSYYHPMDYWGQGTSVKVSS

SEQ ID NO: 206 - protein, 121 aa, synthetic construct
EVQLQQFGAELVKPGTSVKISCKASGYTFTDYNMDWVKQGHGKCLEWIGDINPNYDTTSYSQKFKGKATLTVDKSSSTAYMELRSLTSEDSAVYYCARSEAGNYYWYFDVWGAGTTVKVSS

SEQ ID NO: 207 - protein, 123 aa, synthetic construct
EVQLVQSGAERKKPGSSVKVSCKASGYIFTDYNMDWVRQAPGQCLEWIGDINPDYDTTSYNEKFKGRATLTVDKSTSTAYMELSSLRSEDAAVYYCARGGYSGSSYYHPMDYWGQGTLVTVSS

SEQ ID NO: 208 - protein, 123 aa, synthetic construct
EVQLVQSGAERKKPGSSVKVSCKASGYIFTAYNMDWVRQAPGQCLEWIGDINPDYDTTSYNEKFKGRATLTVDKSTSTAYMELSSLRSEDAAVYYCARGGYSGSSYYHPMDYWGQGTLVTVSS

SEQ ID NO: 209 - protein, 123 aa, synthetic construct
EVQLVQSGAERKKPGSSVKVSCKASGYIFTDANMDWVRQAPGQCLEWIGDINPDYDTTSYNEKFKGRATLTVDKSTSTAYMELSSLRSEDAAVYYCARGGYSGSSYYHPMDYWGQGTLVTVSS

SEQ ID NO: 210 - protein, 123 aa, synthetic construct
EVQLVQSGAERKKPGSSVKVSCKASGYIFTDYNMAWVRQAPGQCLEWIGDINPDYDTTSYNEKFKGRATLTVDKSTSTAYMELSSLRSEDAAVYYCARGGYSGSSYYHPMDYWGQGTLVTVSS

SEQ ID NO: 211 - protein, 123 aa, synthetic construct
EVQLVQSGAERKKPGSSVKVSCKASGYIFTDYNMDWVRQAPGQCLEWIGAINPDYDTTSYNEKFKGRATLTVDKSTSTAYMELSSLRSEDAAVYYCARGGYSGSSYYHPMDYWGQGTLVTVSS

SEQ ID NO: 212 - protein, 123 aa, synthetic construct
EVQLVQSGAERKKPGSSVKVSCKASGYIFTDYNMDWVRQAPGQCLEWIGDINPAYDTTSYNEKFKGRATLTVDKSTSTAYMELSSLRSEDAAVYYCARGGYSGSSYYHPMDYWGQGTLVTVSS

SEQ ID NO: 213 - protein, 123 aa, synthetic construct
EVQLVQSGAERKKPGSSVKVSCKASGYIFTDYNMDWVRQAPGQCLEWIGDINPDADTTSYNEKFKGRATLTVDKSTSTAYMELSSLRSEDAAVYYCARGGYSGSSYYHPMDYWGQGTLVTVSS

SEQ ID NO: 214 - protein, 123 aa, synthetic construct
EVQLVQSGAERKKPGSSVKVSCKASGYIFTDYNMDWVRQAPGQCLEWIGDINPDYATTSYNEKFKGRATLTVDKSTSTAYMELSSLRSEDAAVYYCARGGYSGSSYYHPMDYWGQGTLVTVSS

SEQ ID NO: 215 - protein, 123 aa, synthetic construct
EVQLVQSGAERKKPGSSVKVSCKASGYIFTDYNMDWVRQAPGQCLEWIGDINPDYDTTSANEKFKGRATLTVDKSTSTAYMELSSLRSEDAAVYYCARGGYSGSSYYHPMDYWGQGTLVTVSS

SEQ ID NO: 216 - protein, 123 aa, synthetic construct
EVQLVQSGAERKKPGSSVKVSCKASGYIFTDYNMDWVRQAPGQCLEWIGDINPDYDTTSYNEKFKGRATLTVDKSTSTAYMELSSLRSEDAAVYYCARGGASGSSYYHPMDYWGQGTLVTVSS

SEQ ID NO: 217 - protein, 123 aa, synthetic construct
EVQLVQSGAERKKPGSSVKVSCKASGYIFTDYNMDWVRQAPGQCLEWIGDINPDYDTTSYNEKFKGRATLTVDKSTSTAYMELSSLRSEDAAVYYCARGGYSGSSAYHPMDYWGQGTLVTVSS

SEQ ID NO: 218 - protein, 123 aa, synthetic construct
EVQLVQSGAERKKPGSSVKVSCKASGYIFTDYNMDWVRQAPGQCLEWIGDINPDYDTTSYNEKFKGRATLTVDKSTSTAYMELSSLRSEDAAVYYCARGGYSGSSYAHPMDYWGQGTLVTVSS

SEQ ID NO: 219 - protein, 123 aa, synthetic construct
EVQLVQSGAERKKPGSSVKVSCKASGYIFTDYNMDWVRQAPGQCLEWIGDINPDYDTTSYNEKFKGRATLTVDKSTSTAYMELSSLRSEDAAVYYCARGGYSGSSYYHPMAYWGQGTLVTVSS

SEQ ID NO: 220 - protein, 123 aa, synthetic construct
EVQLVQSGAERKKPGSSVKVSCKASGYIFTDYNMDWVRQAPGQCLEWIGDINPDYDTTSYNEKFKGRATLTVDKSTSTAYMELSSLRSEDAAVYYCARGGYSGSSYYHPMDAWGQGTLVTVSS

SEQ ID NO: 221 - protein, 123 aa, synthetic construct
EVQLQQFGVERVKPGASVKISCKASGYIFTDYNMDWVKQSHGKCLEWIGDINPDYDTTSYNEKFKGKATLTVDKSSSTAYMELRSLTSEDAAVYYCARGGYSGSSYYHPMDYWGQGTSVTVSS

SEQ ID NO: 222 - protein, 121 aa, synthetic construct
EVQLQQFGAERVKPGTSVKISCKASGYTFTDYNMDWVKQGHGKCLEWIGDINPNYDTTSYSQKFKGKATLTVDKSSSTAYMELRSLTSEDAAVYYCARSEAGNYYWYFDVWGAGTTVTVSS

SEQ ID NO: 223 - protein, 118 aa, synthetic construct
QVQLQQSGAERVRPGTSVKISCKASGYAFTNYWLGWVKQRPGHCLEWIGDIFPGSDNTYYNEKFKGKATLTADKSSSTAYMQLSSLASEDAAVYFCARSGNFYAMDYWGQGTSVTVSS

SEQ ID NO: 224 - protein, 123 aa, synthetic construct
EVQLVQSGAERKKPGSSVKVSCKASGYIFTDYNMDWVRQAPGQCLEWIGDINPDYDTTSYNEKFKGRATLTVDKSTSTAYMELSSLRSEDTAVYYCARGGYSGSSYYHPMDYWGQGTLVTVSS

SEQ ID NO: 225 - protein, 123 aa, synthetic construct
EVQLVQSGAERKKPGSSVKVSCKASGYIFTAYNMDWVRQAPGQCLEWIGDINPDYDTTSYNEKFKGRATLTVDKSTSTAYMELSSLRSEDTAVYYCARGGYSGSSYYHPMDYWGQGTLVTVSS

SEQ ID NO: 226 - protein, 123 aa, synthetic construct
EVQLVQSGAERKKPGSSVKVSCKASGYIFTDANMDWVRQAPGQCLEWIGDINPDYDTTSYNEKFKGRATLTVDKSTSTAYMELSSLRSEDTAVYYCARGGYSGSSYYHPMDYWGQGTLVTVSS

SEQ ID NO: 227 - protein, 123 aa, synthetic construct
EVQLVQSGAERKKPGSSVKVSCKASGYIFTDYNMAWVRQAPGQCLEWIGDINPDYDTTSYNEKFKGRATLTVDKSTSTAYMELSSLRSEDTAVYYCARGGYSGSSYYHPMDYWGQGTLVTVSS

SEQ ID NO: 228 - protein, 123 aa, synthetic construct
EVQLVQSGAERKKPGSSVKVSCKASGYIFTDYNMDWVRQAPGQCLEWIGAINPDYDTTSYNEKFKGRATLTVDKSTSTAYMELSSLRSEDTAVYYCARGGYSGSSYYHPMDYWGQGTLVTVSS

SEQ ID NO: 229 - protein, 123 aa, synthetic construct
EVQLVQSGAERKKPGSSVKVSCKASGYIFTDYNMDWVRQAPGQCLEWIGDINPAYDTTSYNEKFKGRATLTVDKSTSTAYMELSSLRSEDTAVYYCARGGYSGSSYYHPMDYWGQGTLVTVSS

SEQ ID NO: 230 - protein, 123 aa, synthetic construct
EVQLVQSGAERKKPGSSVKVSCKASGYIFTDYNMDWVRQAPGQCLEWIGDINPDADTTSYNEKFKGRATLTVDKSTSTAYMELSSLRSEDTAVYYCARGGYSGSSYYHPMDYWGQGTLVTVSS

SEQ ID NO: 231 - protein, 123 aa, synthetic construct
EVQLVQSGAERKKPGSSVKVSCKASGYIFTDYNMDWVRQAPGQCLEWIGDINPDYATTSYNEKFKGRATLTVDKSTSTAYMELSSLRSEDTAVYYCARGGYSGSSYYHPMDYWGQGTLVTVSS

SEQ ID NO: 232 - protein, 123 aa, synthetic construct
EVQLVQSGAERKKPGSSVKVSCKASGYIFTDYNMDWVRQAPGQCLEWIGDINPDYDTTSANEKFKGRATLTVDKSTSTAYMELSSLRSEDTAVYYCARGGYSGSSYYHPMDYWGQGTLVTVSS

SEQ ID NO: 233 - protein, 123 aa, synthetic construct
EVQLVQSGAERKKPGSSVKVSCKASGYIFTDYNMDWVRQAPGQCLEWIGDINPDYDTTSYNEKFKGRATLTVDKSTSTAYMELSSLRSEDTAVYYCARGGASGSSYYHPMDYWGQGTLVTVSS

SEQ ID NO: 234 - protein, 123 aa, synthetic construct
EVQLVQSGAERKKPGSSVKVSCKASGYIFTDYNMDWVRQAPGQCLEWIGDINPDYDTTSYNEKFKGRATLTVDKSTSTAYMELSSLRSEDTAVYYCARGGYSGSSAYHPMDYWGQGTLVTVSS

SEQ ID NO: 235 - protein, 123 aa, synthetic construct
EVQLVQSGAERKKPGSSVKVSCKASGYIFTDYNMDWVRQAPGQCLEWIGDINPDYDTTSYNEKFKGRATLTVDKSTSTAYMELSSLRSEDTAVYYCARGGYSGSSYAHPMDYWGQGTLVTVSS

SEQ ID NO: 236 - protein, 123 aa, synthetic construct
EVQLVQSGAERKKPGSSVKVSCKASGYIFTDYNMDWVRQAPGQCLEWIGDINPDYDTTSYNEKFKGRATLTVDKSTSTAYMELSSLRSEDTAVYYCARGGYSGSSYYHPMAYWGQGTLVTVSS

SEQ ID NO: 237 - protein, 123 aa, synthetic construct
EVQLVQSGAERKKPGSSVKVSCKASGYIFTDYNMDWVRQAPGQCLEWIGDINPDYDTTSYNEKFKGRATLTVDKSTSTAYMELSSLRSEDTAVYYCARGGYSGSSYYHPMDAWGQGTLVTVSS

SEQ ID NO: 238 - protein, 123 aa, synthetic construct
EVQLQQFGVERVKPGASVKISCKASGYIFTDYNMDWVKQSHGKCLEWIGDINPDYDTTSYNEKFKGKATLTVDKSSSTAYMELRSLTSEDTAVYYCARGGYSGSSYYHPMDYWGQGTSVTVSS

SEQ ID NO: 239 - protein, 121 aa, synthetic construct
EVQLQQFGAERVKPGTSVKISCKASGYTFTDYNMDWVKQGHGKCLEWIGDINPNYDTTSYSQKFKGKATLTVDKSSSTAYMELRSLTSEDTAVYYCARSEAGNYYWYFDVWGAGTTVTVSS

SEQ ID NO: 240 - protein, 118 aa, synthetic construct
QVQLQQSGAERVRPGTSVKISCKASGYAFTNYWLGWVKQRPGHCLEWIGDIFPGSDNTYYNEKFKGKATLTADKSSSTAYMQLSSLASEDSAVYFCARSGNFYAMDYWGQGTSVTVSS

SEQ ID NO: 241 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDAAVYYCARGGYSGSSYYHPMDYWGQGTLVTVSS

SEQ ID NO: 242 - protein, 123 aa, synthetic construct
EVQLVQSGAEVKKPGSSVKVSCKASGYIFTAYNMDWVRQAPGQCLEWIGDINPDYDTTSYNEKFKGRATLTVDKSTSTAYMELSSLRSEDAAVYYCARGGYSGSSYYHPMDYWGQGTLVTVSS

SEQ ID NO: 243 - protein, 123 aa, synthetic construct
EVQLVQSGAEVKKPGSSVKVSCKASGYIFTDANMDWVRQAPGQCLEWIGDINPDYDTTSYNEKFKGRATLTVDKSTSTAYMELSSLRSEDAAVYYCARGGYSGSSYYHPMDYWGQGTLVTVSS

SEQ ID NO: 244 - protein, 123 aa, synthetic construct
EVQLVQSGAEVKKPGSSVKVSCKASGYIFTDYNMAWVRQAPGQCLEWIGDINPDYDTTSYNEKFKGRATLTVDKSTSTAYMELSSLRSEDAAVYYCARGGYSGSSYYHPMDYWGQGTLVTVSS

SEQ ID NO: 245 - protein, 123 aa, synthetic construct
EVQLVQSGAEVKKPGSSVKVSCKASGYIFTDYNMDWVRQAPGQCLEWIGAINPDYDTTSYNEKFKGRATLTVDKSTSTAYMELSSLRSEDAAVYYCARGGYSGSSYYHPMDYWGQGTLVTVSS

SEQ ID NO: 246 - protein, 123 aa, synthetic construct
EVQLVQSGAEVKKPGSSVKVSCKASGYIFTDYNMDWVRQAPGQCLEWIGDINPAYDTTSYNEKFKGRATLTVDKSTSTAYMELSSLRSEDAAVYYCARGGYSGSSYYHPMDYWGQGTLVTVSS

SEQ ID NO: 247 - protein, 123 aa, synthetic construct
EVQLVQSGAEVKKPGSSVKVSCKASGYIFTDYNMDWVRQAPGQCLEWIGDINPDADTTSYNEKFKGRATLTVDKSTSTAYMELSSLRSEDAAVYYCARGGYSGSSYYHPMDYWGQGTLVTVSS

SEQ ID NO: 248 - protein, 123 aa, synthetic construct
EVQLVQSGAEVKKPGSSVKVSCKASGYIFTDYNMDWVRQAPGQCLEWIGDINPDYATTSYNEKFKGRATLTVDKSTSTAYMELSSLRSEDAAVYYCARGGYSGSSYYHPMDYWGQGTLVTVSS

SEQ ID NO: 249 - protein, 123 aa, synthetic construct
EVQLVQSGAEVKKPGSSVKVSCKASGYIFTDYNMDWVRQAPGQCLEWIGDINPDYDTTSANEKFKGRATLTVDKSTSTAYMELSSLRSEDAAVYYCARGGYSGSSYYHPMDYWGQGTLVTVSS

SEQ ID NO: 250 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDAAVYYCARGGASGSSYYHPMDYWGQGTLVTVSS

SEQ ID NO: 251 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDAAVYYCARGGYSGSSAYHPMDYWGQGTLVTVSS

SEQ ID NO: 252 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDAAVYYCARGGYSGSSYAHPMDYWGQGTLVTVSS

SEQ ID NO: 253 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDAAVYYCARGGYSGSSYYHPMAYWGQGTLVTVSS

SEQ ID NO: 254 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDAAVYYCARGGYSGSSYYHPMDAWGQGTLVTVSS

SEQ ID NO: 255 - protein, 123 aa, synthetic construct
EVQLQQFGVELVKPGASVKISCKASGYIFTDYNMDWVKQSHGKCLEWIGDINPDYDTTSYNEKFKGKATLTVDKSSSTAYMELRSLTSEDAAVYYCARGGYSGSSYYHPMDYWGQGTSVTVSS

SEQ ID NO: 256 - protein, 121 aa, synthetic construct
EVQLQQFGAELVKPGTSVKISCKASGYTFTDYNMDWVKQGHGKCLEWIGDINPNYDTTSYSQKFKGKATLTVDKSSSTAYMELRSLTSEDAAVYYCARSEAGNYYWYFDVWGAGTTVTVSS

SEQ ID NO: 257 - protein, 118 aa, synthetic construct
QVQLQQSGAELVRPGTSVKISCKASGYAFTNYWLGWVKQRPGHCLEWIGDIFPGSDNTYYNEKFKGKATLTADKSSSTAYMQLSSLASEDAAVYFCARSGNFYAMDYWGQGTSVTVSS

SEQ ID NO: 258 - protein, 123 aa, synthetic construct
EVQLVQSGAEVKKPGESVKVSCKASGYIFTDYNMDWVRQAPGQCLEWIGDINPDYDTTSYNEKFKGRATLTVDKSTSTAYMELSSLRSEDTAVYYCARGGYSGSSYYHPMDYWGQGTSVTVSS

SEQ ID NO: 259 - protein, 123 aa, synthetic construct
EVQLVQSGAEVKKPGESVKVSCKASGYIFTAYNMDWVRQAPGQCLEWIGDINPDYDTTSYNEKFKGRATLTVDKSTSTAYMELSSLRSEDTAVYYCARGGYSGSSYYHPMDYWGQGTSVTVSS

SEQ ID NO: 260 - protein, 123 aa, synthetic construct
EVQLVQSGAEVKKPGESVKVSCKASGYIFTDANMDWVRQAPGQCLEWIGDINPDYDTTSYNEKFKGRATLTVDKSTSTAYMELSSLRSEDTAVYYCARGGYSGSSYYHPMDYWGQGTSVTVSS

SEQ ID NO: 261 - protein, 123 aa, synthetic construct
EVQLVQSGAEVKKPGESVKVSCKASGYIFTDYNMAWVRQAPGQCLEWIGDINPDYDTTSYNEKFKGRATLTVDKSTSTAYMELSSLRSEDTAVYYCARGGYSGSSYYHPMDYWGQGTSVTVSS

SEQ ID NO: 262 - protein, 123 aa, synthetic construct
EVQLVQSGAEVKKPGESVKVSCKASGYIFTDYNMDWVRQAPGQCLEWIGAINPDYDTTSYNEKFKGRATLTVDKSTSTAYMELSSLRSEDTAVYYCARGGYSGSSYYHPMDYWGQGTSVTVSS

SEQ ID NO: 263 - protein, 123 aa, synthetic construct
EVQLVQSGAEVKKPGESVKVSCKASGYIFTDYNMDWVRQAPGQCLEWIGDINPAYDTTSYNEKFKGRATLTVDKSTSTAYMELSSLRSEDTAVYYCARGGYSGSSYYHPMDYWGQGTSVTVSS

SEQ ID NO: 264 - protein, 123 aa, synthetic construct
EVQLVQSGAEVKKPGESVKVSCKASGYIFTDYNMDWVRQAPGQCLEWIGDINPDADTTSYNEKFKGRATLTVDKSTSTAYMELSSLRSEDTAVYYCARGGYSGSSYYHPMDYWGQGTSVTVSS

SEQ ID NO: 265 - protein, 123 aa, synthetic construct
EVQLVQSGAEVKKPGESVKVSCKASGYIFTDYNMDWVRQAPGQCLEWIGDINPDYATTSYNEKFKGRATLTVDKSTSTAYMELSSLRSEDTAVYYCARGGYSGSSYYHPMDYWGQGTSVTVSS

SEQ ID NO: 266 - protein, 123 aa, synthetic construct
EVQLVQSGAEVKKPGESVKVSCKASGYIFTDYNMDWVRQAPGQCLEWIGDINPDYDTTSANEKFKGRATLTVDKSTSTAYMELSSLRSEDTAVYYCARGGYSGSSYYHPMDYWGQGTSVTVSS

SEQ ID NO: 267 - protein, 123 aa, synthetic construct
EVQLVQSGAEVKKPGESVKVSCKASGYIFTDYNMDWVRQAPGQCLEWIGDINPDYDTTSYNEKFKGRATLTVDKSTSTAYMELSSLRSEDTAVYYCARGGASGSSYYHPMDYWGQGTSVTVSS

SEQ ID NO: 268 - protein, 123 aa, synthetic construct
EVQLVQSGAEVKKPGESVKVSCKASGYIFTDYNMDWVRQAPGQCLEWIGDINPDYDTTSYNEKFKGRATLTVDKSTSTAYMELSSLRSEDTAVYYCARGGYSGSSAYHPMDYWGQGTSVTVSS

SEQ ID NO: 269 - protein, 123 aa, synthetic construct
EVQLVQSGAEVKKPGESVKVSCKASGYIFTDYNMDWVRQAPGQCLEWIGDINPDYDTTSYNEKFKGRATLTVDKSTSTAYMELSSLRSEDTAVYYCARGGYSGSSYAHPMDYWGQGTSVTVSS

SEQ ID NO: 270 - protein, 123 aa, synthetic construct
EVQLVQSGAEVKKPGESVKVSCKASGYIFTDYNMDWVRQAPGQCLEWIGDINPDYDTTSYNEKFKGRATLTVDKSTSTAYMELSSLRSEDTAVYYCARGGYSGSSYYHPMAYWGQGTSVTVSS

SEQ ID NO: 271 - protein, 123 aa, synthetic construct
EVQLVQSGAEVKKPGESVKVSCKASGYIFTDYNMDWVRQAPGQCLEWIGDINPDYDTTSYNEKFKGRATLTVDKSTSTAYMELSSLRSEDTAVYYCARGGYSGSSYYHPMDAWGQGTSVTVSS

SEQ ID NO: 272 - protein, 121 aa, synthetic construct
EVQLQQFGAELVKPGESVKISCKASGYTFTDYNMDWVKQGHGKCLEWIGDINPNYDTTSYSQKFKGKATLTVDKSSSTAYMELRSLTSEDTAVYYCARSEAGNYYWYFDVWGAGTSVTVSS

SEQ ID NO: 273 - protein, 123 aa, synthetic construct
EVQLVQSGAEVKKPGESVKVSCKASGYIFTDYNMDWVRQAPGQCLEWIGDINPDYDTTSYNEKFKGRATLTVDKSTSTAYMELSSLRSEDTAVYYCARGGYSGSSYYHPMDYWGQGTLVTVSS

SEQ ID NO: 274 - protein, 123 aa, synthetic construct
EVQLVQSGAEVKKPGESVKVSCKASGYIFTAYNMDWVRQAPGQCLEWIGDINPDYDTTSYNEKFKGRATLTVDKSTSTAYMELSSLRSEDTAVYYCARGGYSGSSYYHPMDYWGQGTLVTVSS

SEQ ID NO: 275 - protein, 123 aa, synthetic construct
EVQLVQSGAEVKKPGESVKVSCKASGYIFTDANMDWVRQAPGQCLEWIGDINPDYDTTSYNEKFKGRATLTVDKSTSTAYMELSSLRSEDTAVYYCARGGYSGSSYYHPMDYWGQGTLVTVSS

SEQ ID NO: 276 - protein, 123 aa, synthetic construct
EVQLVQSGAEVKKPGESVKVSCKASGYIFTDYNMAWVRQAPGQCLEWIGDINPDYDTTSYNEKFKGRATLTVDKSTSTAYMELSSLRSEDTAVYYCARGGYSGSSYYHPMDYWGQGTLVTVSS

SEQ ID NO: 277 - protein, 123 aa, synthetic construct
EVQLVQSGAEVKKPGESVKVSCKASGYIFTDYNMDWVRQAPGQCLEWIGAINPDYDTTSYNEKFKGRATLTVDKSTSTAYMELSSLRSEDTAVYYCARGGYSGSSYYHPMDYWGQGTLVTVSS

SEQ ID NO: 278 - protein, 123 aa, synthetic construct
EVQLVQSGAEVKKPGESVKVSCKASGYIFTDYNMDWVRQAPGQCLEWIGDINPAYDTTSYNEKFKGRATLTVDKSTSTAYMELSSLRSEDTAVYYCARGGYSGSSYYHPMDYWGQGTLVTVSS

SEQ ID NO: 279 - protein, 123 aa, synthetic construct
EVQLVQSGAEVKKPGESVKVSCKASGYIFTDYNMDWVRQAPGQCLEWIGDINPDADTTSYNEKFKGRATLTVDKSTSTAYMELSSLRSEDTAVYYCARGGYSGSSYYHPMDYWGQGTLVTVSS

SEQ ID NO: 280 - protein, 123 aa, synthetic construct
EVQLVQSGAEVKKPGESVKVSCKASGYIFTDYNMDWVRQAPGQCLEWIGDINPDYATTSYNEKFKGRATLTVDKSTSTAYMELSSLRSEDTAVYYCARGGYSGSSYYHPMDYWGQGTLVTVSS

SEQ ID NO: 281 - protein, 123 aa, synthetic construct
EVQLVQSGAEVKKPGESVKVSCKASGYIFTDYNMDWVRQAPGQCLEWIGDINPDYDTTSANEKFKGRATLTVDKSTSTAYMELSSLRSEDTAVYYCARGGYSGSSYYHPMDYWGQGTLVTVSS

SEQ ID NO: 282 - protein, 123 aa, synthetic construct
EVQLVQSGAEVKKPGESVKVSCKASGYIFTDYNMDWVRQAPGQCLEWIGDINPDYDTTSYNEKFKGRATLTVDKSTSTAYMELSSLRSEDTAVYYCARGGASGSSYYHPMDYWGQGTLVTVSS

SEQ ID NO: 283 - protein, 123 aa, synthetic construct
EVQLVQSGAEVKKPGESVKVSCKASGYIFTDYNMDWVRQAPGQCLEWIGDINPDYDTTSYNEKFKGRATLTVDKSTSTAYMELSSLRSEDTAVYYCARGGYSGSSAYHPMDYWGQGTLVTVSS

SEQ ID NO: 284 - protein, 123 aa, synthetic construct
EVQLVQSGAEVKKPGESVKVSCKASGYIFTDYNMDWVRQAPGQCLEWIGDINPDYDTTSYNEKFKGRATLTVDKSTSTAYMELSSLRSEDTAVYYCARGGYSGSSYAHPMDYWGQGTLVTVSS

SEQ ID NO: 285 - protein, 123 aa, synthetic construct
EVQLVQSGAEVKKPGESVKVSCKASGYIFTDYNMDWVRQAPGQCLEWIGDINPDYDTTSYNEKFKGRATLTVDKSTSTAYMELSSLRSEDTAVYYCARGGYSGSSYYHPMAYWGQGTLVTVSS

SEQ ID NO: 286 - protein, 123 aa, synthetic construct
EVQLVQSGAEVKKPGESVKVSCKASGYIFTDYNMDWVRQAPGQCLEWIGDINPDYDTTSYNEKFKGRATLTVDKSTSTAYMELSSLRSEDTAVYYCARGGYSGSSYYHPMDAWGQGTLVTVSS

SEQ ID NO: 287 - protein, 123 aa, synthetic construct
EVQLQQFGVELVKPGESVKISCKASGYIFTDYNMDWVKQSHGKCLEWIGDINPDYDTTSYNEKFKGKATLTVDKSSSTAYMELRSLTSEDTAVYYCARGGYSGSSYYHPMDYWGQGTSVTVSS

SEQ ID NO: 288 - protein, 121 aa, synthetic construct
EVQLQQFGAELVKPGESVKISCKASGYTFTDYNMDWVKQGHGKCLEWIGDINPNYDTTSYSQKFKGKATLTVDKSSSTAYMELRSLTSEDTAVYYCARSEAGNYYWYFDVWGAGTLVTVSS

SEQ ID NO: 289 - protein, 118 aa, synthetic construct
QVQLQQSGAELVRPGESVKISCKASGYAFTNYWLGWVKQRPGHCLEWIGDIFPGSDNTYYNEKFKGKATLTADKSSSTAYMQLSSLASEDSAVYFCARSGNFYAMDYWGQGTSVTVSS

SEQ ID NO: 290 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDTAVYYCARGGYSGSSYYHPMDYWGQGTSVTVSS

SEQ ID NO: 291 - protein, 123 aa, synthetic construct
EVQLVQSGAEVKKPGSSVKVSCKASGYIFTAYNMDWVRQAPGQCLEWIGDINPDYDTTSYNEKFKGRATLTVDKSTSTAYMELSSLRSEDTAVYYCARGGYSGSSYYHPMDYWGQGTSVTVSS

SEQ ID NO: 292 - protein, 123 aa, synthetic construct
EVQLVQSGAEVKKPGSSVKVSCKASGYIFTDANMDWVRQAPGQCLEWIGDINPDYDTTSYNEKFKGRATLTVDKSTSTAYMELSSLRSEDTAVYYCARGGYSGSSYYHPMDYWGQGTSVTVSS

SEQ ID NO: 293 - protein, 123 aa, synthetic construct
EVQLVQSGAEVKKPGSSVKVSCKASGYIFTDYNMAWVRQAPGQCLEWIGDINPDYDTTSYNEKFKGRATLTVDKSTSTAYMELSSLRSEDTAVYYCARGGYSGSSYYHPMDYWGQGTSVTVSS

SEQ ID NO: 294 - protein, 123 aa, synthetic construct
EVQLVQSGAEVKKPGSSVKVSCKASGYIFTDYNMDWVRQAPGQCLEWIGAINPDYDTTSYNEKFKGRATLTVDKSTSTAYMELSSLRSEDTAVYYCARGGYSGSSYYHPMDYWGQGTSVTVSS

SEQ ID NO: 295 - protein, 123 aa, synthetic construct
EVQLVQSGAEVKKPGSSVKVSCKASGYIFTDYNMDWVRQAPGQCLEWIGDINPAYDTTSYNEKFKGRATLTVDKSTSTAYMELSSLRSEDTAVYYCARGGYSGSSYYHPMDYWGQGTSVTVSS

SEQ ID NO: 296 - protein, 123 aa, synthetic construct
EVQLVQSGAEVKKPGSSVKVSCKASGYIFTDYNMDWVRQAPGQCLEWIGDINPDADTTSYNEKFKGRATLTVDKSTSTAYMELSSLRSEDTAVYYCARGGYSGSSYYHPMDYWGQGTSVTVSS

SEQ ID NO: 297 - protein, 123 aa, synthetic construct
EVQLVQSGAEVKKPGSSVKVSCKASGYIFTDYNMDWVRQAPGQCLEWIGDINPDYATTSYNEKFKGRATLTVDKSTSTAYMELSSLRSEDTAVYYCARGGYSGSSYYHPMDYWGQGTSVTVSS

SEQ ID NO: 298 - protein, 123 aa, synthetic construct
EVQLVQSGAEVKKPGSSVKVSCKASGYIFTDYNMDWVRQAPGQCLEWIGDINPDYDTTSANEKFKGRATLTVDKSTSTAYMELSSLRSEDTAVYYCARGGYSGSSYYHPMDYWGQGTSVTVSS

SEQ ID NO: 299 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDTAVYYCARGGASGSSYYHPMDYWGQGTSVTVSS

SEQ ID NO: 300 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDTAVYYCARGGYSGSSAYHPMDYWGQGTSVTVSS

SEQ ID NO: 301 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDTAVYYCARGGYSGSSYAHPMDYWGQGTSVTVSS

SEQ ID NO: 302 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDTAVYYCARGGYSGSSYYHPMAYWGQGTSVTVSS

SEQ ID NO: 303 - protein, 123 aa, synthetic construct
EVQLVQSGAEVKKPGSSVKVSCKASGYIFTDYNMDWVRQAPGQCLEWIGDINPDYDTTSYNEKFKGRATLTVDKSTSTAYMELSSLRSEDTAVYYCARGGYSGSSYYHPMDAWGQGTSVTVSS

SEQ ID NO: 304 - protein, 121 aa, synthetic construct
EVQLQQFGAELVKPGTSVKISCKASGYTFTDYNMDWVKQGHGKCLEWIGDINPNYDTTSYSQKFKGKATLTVDKSSSTAYMELRSLTSEDTAVYYCARSEAGNYYWYFDVWGAGTSVTVSS

SEQ ID NO: 305 - protein, 256 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGCGTKVEIKGGGGSGGGGSGGGGSGGGGSEVQLVQSGAEVKKPGSSVKVSCKASGYIFTDYNMDWVRQAPGQCLEWIGDINPDADTTSYNEKFKGRATLTVDKSTSTAYMELSSLRSEDTAVYYCARGGYSGSSYYHPMDYWGQGTLVTVKS

SEQ ID NO: 306 - protein, 256 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGCGTKVEIKGGGGSGGGGSGGGGSGGGGSEVQLVQSGAEVKKPGSSVKVSCKASGYIFTDYNMDWVRQAPGQCLEWIGDINPDADTTSYNEKFKGRATLTVDKSTSTAYMELSSLRSEDTAVYYCARGGYSGSSYYHPMDYWGQGTLVKVSS

SEQ ID NO: 307 - protein, 256 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGCGTKVEIKGGGGSGGGGSGGGGSGGGGSEVQLVQSGAEVKKPGSSVKVSCKASGYIFTDYNMDWVRQAPGQCLEWIGDINPDADTTSYNEKFKGRATLTVDKSTSTAYMELSSLRSEDSAVYYCARGGYSGSSYYHPMDYWGQGTLVTVKS

SEQ ID NO: 308 - protein, 256 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGCGTKVEIKGGGGSGGGGSGGGGSGGGGSEVQLVQSGAEVKKPGSSVKVSCKASGYIFTDYNMDWVRQAPGQCLEWIGDINPDADTTSYNEKFKGRATLTVDKSTSTAYMELSSLRSEDSAVYYCARGGYSGSSYYHPMDYWGQGTLVKVSS

SEQ ID NO: 309 - protein, 256 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGCGTKVEIKGGGGSGGGGSGGGGSGGGGSEVQLVQSGAERKKPGSSVKVSCKASGYIFTDYNMDWVRQAPGQCLEWIGDINPDADTTSYNEKFKGRATLTVDKSTSTAYMELSSLRSEDTAVYYCARGGYSGSSYYHPMDYWGQGTLVKVSS

SEQ ID NO: 310 - protein, 256 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGCGTKVEIKGGGGSGGGGSGGGGSGGGGSEVQLVQSGAERKKPGSSVKVSCKASGYIFTDYNMDWVRQAPGQCLEWIGDINPDADTTSYNEKFKGRATLTVDKSTSTAYMELSSLRSEDAAVYYCARGGYSGSSYYHPMDYWGQGTLVTVSS

SEQ ID NO: 311 - protein, 256 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGCGTKVEIKGGGGSGGGGSGGGGSGGGGSEVQLVQSGAEVKKPGSSVKVSCKASGYIFTDYNMDWVRQAPGQCLEWIGDINPDADTTSYNEKFKGRATLTVDKSTSTAYMELSSLRSEDAAVYYCARGGYSGSSYYHPMDYWGQGTLVTVSS

SEQ ID NO: 312 - protein, 256 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGCGTKVEIKGGGGSGGGGSGGGGSGGGGSEVQLVQSGAEVKKPGESVKVSCKASGYIFTDYNMDWVRQAPGQCLEWIGDINPDADTTSYNEKFKGRATLTVDKSTSTAYMELSSLRSEDTAVYYCARGGYSGSSYYHPMDYWGQGTSVTVSS

SEQ ID NO: 313 - protein, 256 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGCGTKVEIKGGGGSGGGGSGGGGSGGGGSEVQLVQSGAEVKKPGESVKVSCKASGYIFTDYNMDWVRQAPGQCLEWIGDINPDADTTSYNEKFKGRATLTVDKSTSTAYMELSSLRSEDTAVYYCARGGYSGSSYYHPMDYWGQGTLVTVSS

SEQ ID NO: 314 - protein, 256 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGCGTKVEIKGGGGSGGGGSGGGGSGGGGSEVQLVQSGAEVKKPGSSVKVSCKASGYIFTDYNMDWVRQAPGQCLEWIGDINPDADTTSYNEKFKGRATLTVDKSTSTAYMELSSLRSEDTAVYYCARGGYSGSSYYHPMDYWGQGTSVTVSS

SEQ ID NO: 315 - protein, 256 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGCGTKVEIKGGGGSGGGGSGGGGSGGGGSEVQLVQSGAERKKPGSSVKVSCKASGYIFTDYNMDWVRQAPGQCLEWIGDINPDADTTSYNEKFKGRATLTVDKSTSTAYMELSSLRSEDTAVYYCARGGYSGSSYYHPMDYWGQGTLVTVSS